(19) United States
(12) Patent Application Publication
(10) Pub. No.: US 2014/0378513 A1
(43) Pub. Date: Dec. 25, 2014

(54) USE OF FUNGICIDES IN LIQUID SYSTEMS

(71) Applicant: SHAPPHIRE ENERGY, INC., SAN DIEGO, CA (US)

(72) Inventors: Robert C McBride, San Diego, CA (US); Craig A Behnke, San Diego, CA (US); Kyle M Botsch, San Diego, CA (US); Nicole A Heaps, San Diego, CA (US); Christopher Del Meenach, Mesilla Park, NM (US)

(21) Appl. No.: 14/351,540

(22) PCT Filed: Oct. 12, 2012
(86) PCT No.: PCT/US2012/060120

§ 371 Date: Apr. 11, 2014

Related U.S. Application Data
(60) Provisional application No. 61/547,473, filed on Oct. 14, 2011.

(51) Int. Cl. C12N 1/12; A01N 43/56; C12Q 1/68; A01N 43/40

(52) U.S. Cl. CPC C12N 1/12; A01N 43/40; A01N 43/56; C12Q 1/6895; C12Q 2600/16
USPC 514/352; 435/6.12; 506/2; 435/6.15

(57) ABSTRACT

The present disclosure provides methods to detect pests in liquid culture systems for the growth of microalgae. The disclosure further provides methods to treat and control pests in a liquid system and for methods to increase yields of microalgae grown in a liquid culture systems. Methods are provided for the growth, monitoring, treatment and harvesting of microalgae from liquid culture systems.

### CROSS REFERENCE TO RELATED APPLICATIONS

This application claims the benefit of U.S. Provisional Patent Application 61/547,473, filed Oct. 14, 2011, which is incorporated herein by reference in its entirety for all purposes.

### FIELD

This disclosure includes methods that provide increased yields in liquid systems, such as pools and ponds and the like. The disclosure also includes methods for detecting pests in such systems. Such systems are useful for the production of aquatic biomass, such as algae, and in particular microalgae and cyanobacteria. Aquatic biomass produced using the methods described herein can be used to produce a variety of useful products. In one embodiment, the biomass produced is used for the production of oil which can be refined into a variety of products, including, but not limited to, transportation fuels.

### BACKGROUND

Microalgae are unicellular non-vascular photosynthetic organisms, producing oxygen by photosynthesis. One group, the microalgae, are useful for biotechnology applications for many reasons, including their high growth rate and tolerance to varying environmental conditions. Use of microalgae in a variety of industrial processes for commercially important products has been reported. For example, microalgae have uses in the production of nutritional supplements, pharmaceuticals, natural dyes, a food source for fish and crustaceans, biological control of agricultural pests, production of oxygen and removal of nitrogen, phosphorus and toxic substances in sewage treatment, and pollution controls, such as biodegradation of plastics or uptake of carbon dioxide.

Microalgae have received increasing attention for the production of fuel products. Fuel products, such as oil, petrochemicals, and other substances useful for the production of petrochemicals are increasingly in demand.

Microalgae can produce 10 to 100 times as much mass as terrestrial plants in a year. Microalgae also produce oils (lipids), proteins and starches that may be converted into biofuels. These microalgae can grow almost anywhere, though are most commonly found at latitudes between 40 N and 40 S. With more than 100,000 known species of diatoms (a type of microalgae), 40,000 known species of green plant-like microalgae, and smaller numbers of other microalgae species, microalgae will grow rapidly in nearly any environment, with almost any kind of water, including marginal areas with limited or poor quality water.

Microalgae can store energy in the form of either oil or starch. Stored oil can be as much as 60% of the weight of the microalgae. Certain species which are enhanced in oil or starch production have been identified, and growing conditions have been tested. Processes for extracting and converting these materials to fuels have also been developed.

Thus, there exists a pressing need for alternative methods to develop fuel products that are renewable, sustainable, and less harmful to the environment.

### SUMMARY

This disclosure includes a method of reducing the growth of a fungus in a liquid system comprising inoculating the liquid system with a microalgae, detecting the fungus; providing an effective concentration of a fungicide to inhibit the growth of the fungus relative to the growth of the fungus without the fungicide and growing the microalgae.

This disclosure includes a method of reducing the growth of a pest in a liquid system comprising inoculating the liquid system with a microalgae, detecting the pest; providing an effective concentration of a pesticide to inhibit the growth of the pest relative to the growth of the pest without the pesticide and growing the microalgae.

The present disclosure also provides for methods of detecting the presence of a fungus in a liquid system of microalgae comprising obtaining a sample of the liquid system; and detecting the presence of a DNA sequence indicative of a fungus.

The present disclosure also provides for methods of detecting the presence of a pest in a liquid system of microalgae comprising obtaining a sample of the liquid system; and detecting the presence of a DNA sequence indicative of a pest.

The present disclosure provides a method of enhancing a yield of microalgae in a liquid system comprising providing a liquid system comprising a fungicide; and growing a microalgae for at least 10 days in a liquid system in the presence of a fungicide.

The present disclosure further provides a method of enhancing a yield of microalgae in a liquid system comprising providing a liquid system; and growing a microalgae for at least 10 days in a liquid system where one or more fungicides are provided sequentially to suppress the growth of a pest.

In addition, the present disclosure provides a method of preventing the growth of a fungus in a liquid microalgae culture system comprising providing an effective concentration of a fungicide to inhibit the growth of a fungus in a liquid, where the fungicide does not notably inhibit the growth of a microalgae, inoculating the fungicide treated liquid with a microalgae, and growing a microalgae.

The present disclosure also provides for methods of treating a liquid microalgae culture system comprising detecting the presence of a fungus in a liquid system; providing an effective concentration of a fungicide to a liquid system to inhibit the growth of a fungus growing on a microalgae; and monitoring said liquid system at least once for the presence of said fungus.

The present disclosure further provides a liquid microalgae culture system comprising a transgenic microalgae and a fungicide.

The present disclosure further provides for methods of detecting a chytrid comprising obtaining a sample, performing a polymerase chain reaction on a sample using a pair of oligonucleotide primers capable of amplifying a nucleic acid molecule having a sequence selected the group consisting of SEQ ID NOs: 1 to 6, or a complement thereof.

### BRIEF DESCRIPTION OF THE SEQUENCES

SEQ ID NO: 1 sets forth the sequence of the Internal transcribed spacer (ITS) regions and 5.8S RNA of chytrid FD01.

SEQ ID NO: 2 sets forth the sequence of the Internal transcribed spacer (ITS) regions and 5.8S RNA of chytrid FD61.

SEQ ID NO: 3 sets forth the sequence of the Internal transcribed spacer (ITS) regions and 5.8S RNA of chytrid FD95.

SEQ ID NO: 4 sets forth the sequence of the Internal transcribed spacer (ITS) regions and 5.8S RNA of chytrid FD100.

SEQ ID NO: 5 sets forth the sequence of the Internal transcribed spacer (ITS) regions and 5.8S RNA of chytrid FD101.

SEQ ID NO: 6 sets forth the sequence of the Internal transcribed spacer (ITS) regions and 5.8S RNA of chytrid FDARG.

SEQ ID NO: 7 sets forth the sequence of Peptide nucleic acid (PNA) inhibitor sequence SE0004-PNA2.

SEQ ID NO: 8 sets forth the sequence of PNA inhibitor sequence SE0107-PNA2.

SEQ ID NO: 9 sets forth the sequence of PNA inhibitor sequence SE0087-PNA4.

SEQ ID NOs 10 to 37 sets forth oligonucleotide sequences for polymerase chain reaction (PCR) assays.

### BRIEF DESCRIPTION OF THE DRAWINGS

FIG. 1 is a phylogenetic tree presenting the results of a phylogenetic analysis of isolated chytrid pests.

FIG. 2 is a graph of the results of fluorescence measurement of a dilution series of Calcofluor White binding to chytrid infected microalgae culture samples.

FIG. 3 is a graph of the results of chlorophyll fluorescence of a dilution series of chytrid infected microalgae culture samples.

FIG. 4 provides images of Calcofluor White binding to chytrid infected microalgae cultures.

FIG. 5 is a graph of the fluorescence ratios of Calcofluor White to chlorophyll in chytrid infected microalgae samples.

FIG. 6 is a graph of the results of the C_{T} values of chytrid infected microalgae cultures having Calcofluor White fluorescence.

FIG. 7 is a graph showing monitoring of a desmid pond culture for four different chytrids known to be pests of a desmid.

FIG. 8 is a graph showing the effects of the fungicide fluazinam on uncontaminated microalgae growth.

FIG. 9 is a graph showing the growth of a contaminated microalgae culture either with or without fluazinam. (1 ppm=1 mg/L).

FIG. 10 is a graph showing the effect of the fungicide Headline® on the growth of an uncontaminated microalgae.

FIG. 11 is a graph of the growth of a contaminated microalgae culture with or without the fungicide Headline®. (1 ppm=1 mg/L).

FIG. 12 is a graph of the effect of Thiram® on the growth of an uncontaminated microalgae culture. (1 ppm=1 mg/L).

FIG. 13 is a graph of the growth of a contaminated microalgae culture with or without the fungicide Thiram®. (1 ppm=1 mg/L).

FIG. 14 is a graph of the effects of fungicide treatment on the density microalgae P16 growing in an outdoor open pond.

FIG. 15 is a graph showing the growth of a microalgae cultures with or without fungicide treatment.

FIG. 16 is a graph of the growth and harvesting of a microalgae grown in an outdoor open pond.

FIG. 17 is a graph showing the monitoring and treatment of an outdoor microalgae culture.

### DETAILED DESCRIPTION

Unless defined otherwise, technical and scientific terms used herein have the same meaning as commonly understood by one of ordinary skill in the art. One skilled in the art will recognize that many methods can be used in the practice of the present invention. Indeed, the present invention is in no way limited to the methods and materials described. For purposes of the present disclosure, the following terms are defined below.

“Environmental sample” relates to samples obtained from the environment surrounding where algae are being grown, or where algae may be grown. As used herein, an environmental sample may be taken from the air, the soil, the vegetation and the water in the environments mentioned above or in the surrounding area. These samples are collected in accord with standard collection protocols for collecting microbial samples.

“Growing microalgae,” “growing the microalgae,” “microalgae growth,” and “culturing the microalgae” as used herein, refer to one or more steps including microalgae in culture to when microalgae are in suspension just prior to the beginning of a harvesting step.

As used herein, the term “pest” relates to any undesired biological organism in a sample culture. Non-limiting examples of pests are bacteria and fungi. A pest may be undesired because it decreases the growth rate of a microalgae culture. Alternatively, a pest may be undesired because it decreases the overall extent of microalgal growth or the total yield of microalgae per volume of culture. A pest may be undesired because it leads to the death of a microalgae culture. A pest may be undesired because it changes the gene expression of the cultured microalgae. A pest may be population of a single organism or a mixed population.

A “microalgae”, as used herein, is a non-vascular photosynthetic organism, for example, an organism classified as photosynthetic bacteria (including cyanobacteria), cyanophyta, prochlorophyta, rhodophyta, chlorophyta, heterokontophyta, tribophyta, glaucophyta, chlorarachniophytes, euglenophyta, euglenoids, haptophyta, chrysophyta, cryptophyta, cryptomonads, dinophyta, dinoflagellata, desmidiales, pyrmnesiophyta, bacillariophyta, xanthophyta, eustigmatophyta, raphidophyta, phaeophyta, and phytoplankton. A microalgae may also be a microalgae species including, but not limited to, Chlamydomonas. reinhardtii, Dunaliella salina, Nannochloropsis salina, Nannochloropsis occulata, Scenedesmis dimorphus, Scenedesmus obliquus, Dunaliella tertiolecta, or Haematococcus pluvialis. A “microalgae” of the present disclosure may be a unicellular non-vascular photosynthetic organism. In other instances, the microalgae may be one or more cells of a multicellular non-vascular photosynthetic organism.

A “fungus,” as used herein, is a member of the fungi kingdom and the division Blastocladiomycota, Chytridiomycota, Glomeromycota, Microsporidia, Neocallimastigomycota, Ascomycota, or Basidiomycota. A fungus, as used herein, includes members of the classes Chytridiomycetes and Monoblepharidomycetes as well as species of Chytridium spp., or any combination of fungi. A fungus as used herein includes members of Chytridium species included in the Chytridiomycota division of the fungi kingdom including the orders Chytridiales, Rhizophylctidales, Spizellomycetales, Rhizophydiales, Lobulomycetales, Cladochytriales, Polychytrium and Monoblepharidomycetes.

As used herein, “reduced growth,” “inhibited growth,” “growth reduction” and “growth inhibition” relate to the decreased reproduction or division of a pest relative to the amount of reproduction or division of a pest under similar or identical conditions in the absence of any treatment. “Reduced growth,” “inhibited growth,” “growth reduction” and “growth inhibition” may also refer to the killing or death of the pest by the treatment.

As used herein, “harvesting,” relates to the removal or isolation of all, or part of microalgae in a culture system, including a liquid culture system. Harvesting may occur continuously from a growing culture, batchwise or as a total collection of the microalgae at the end of a culture period. A liquid, as a supernate, siphonate, flow-through or other separated form, may be returned to the liquid culture system. Relative amounts harvested refer to the amount of microalgae remaining compared to the amount contained in the liquid culture system before harvesting.

“Recycled liquid” or “returned liquid,” as used herein refers to the liquid remaining after the harvesting or removal of more than 50%, 60%, 70%, 80%, 90%, 95% or all of the microalgae from the liquid culture system.

“Culture time,” or “length of growth,” or “time to harvest,” as used herein is measured from the date of inoculation of a liquid culture system with microalgae.

As used herein, the term “yield” refers to the number of microalgae per unit volume at harvest, and may be expressed, for example, as the number of cells per volume of culture, a mass per volume of culture, etc. Yield, used herein may also be expressed as a mass per area of culture. Changes in yield are expressed as the change, either increase or decrease, in the yield with or without a treatment.

As used herein, the term “liquid system,” “liquid culture system,” or “culture system” refers to a system for culturing a microalgae. A liquid system may include both a closed and an open culture system. An open liquid system may include, for example an open or closed photobioreactor, semi-closed ponds, open ponds, or lakes.

As used herein, the term “treatment” refers to methods or compositions that inhibit the growth of a pest. A treatment may include methods or compositions that kill a pest.

As used herein, the term “effective concentration” refers to a concentration of a pesticide or fungicide that is sufficient to control the growth, or kill, a pest while providing for the continued growth, or survival, of the growing microalgae culture in the liquid system.

The present disclosure provides for methods of reducing the growth of a pest in a liquid culture of microalgae where a liquid system is inoculated with a microalgae, the system is monitored for the presence of a pest, and an effective concentration of a fungicide is provided to inhibit the growth of the pest relative to the growth of the pest without the fungicide, and growing the microalgae. The present disclosure further provides for the reduction of viable pests in a liquid system.

In one aspect, the present disclosure provides for a method of reducing the growth of a pest where a reduction of the growth of a pest in the presence of an inhibitor is measured relative to the growth of a pest under similar conditions in the absence of an inhibitor. In one aspect, a reduction of the growth of a pest is achieved by the death of the pest. In another aspect, a reduction of the growth of a pest is achieved by the inhibition of division of the pest. In an aspect, growth of the pest is reduced by 99%, or more. In another aspect, the growth of the pest is reduced by 95%, or more. In yet another aspect, the growth of a pest is reduced by 90%, or more. In another aspect, the growth of a pest is reduced by at least 80%. In another aspect, the growth of a pest is reduced by at least 70%. In another aspect, the growth of a pest is reduced by at least 60%. In another aspect, the growth of a pest is reduced by at least 50%. In another aspect, the growth of a pest is reduced by at least 90 to 99%, at least 95 to 99%, at least 80 to 95%, at least 80 to 99%, or 75 to 99%. In yet another aspect, the growth of a pest is reduced no less than 90%, 95% or 99%.

In an aspect, the pest may be a member of the fungi kingdom. In another aspect, the pest may be a member of the division Chytridiomycota. In yet another aspect, the pest may be a member of the class Chytridiomycetes. In a further aspect, the pest may be a species of Chytridium spp. In another aspect, the pest may be identifiable by a nucleic acid sequence selected from chytrids identifiable using the nucleic acid sequences selected from the group consisting of SEQ ID NOs: 1-6.

Examples of pests of microalgae cultures are members of the fungi kingdom and include the division Blastocladiomycota, Chytridiomycota, Glomeromycota, Microsporidia, Neocallimastigomycota, Ascomycota, or Basidiomycota. A fungus, as used herein, includes members of the classes Chytridiomyicetes and Monoblepharidomycetes as well as species of Chytridium spp. In an aspect, pests that are members of the fungi kingdom may be identified by molecular phylogeny, for example, using the methods of James et al. “A molecular phylogeny of the flagellated fungi (Chytridiomycota) and description of a new phylum (Blastocladiomycota),” Mycologia 98(6):860-71 (2006), herein incorporated by reference in its entirety.

In another aspect, a pest may be a member of the Rozella genus of Chytridiomycota. In an aspect, a pest may be a member of the Chytridiales/Rhizophydium clade of Chytridiomycota. In yet another aspect, a pest may be a member of the Amoeboaphelidium genus. In a further aspect, a pest may be most closely related phylogenetically to chytrids identifiable by SEQ ID NOs: 1 to 6. In another aspect, a pest may be phylogenetically related to a clade of the Chytriodmycota division including the Chytridiales, Rhizophylctidales, Spizellomycetales, Rhizophydiales, Lobulomycetales, Cladochytriales, Polychytrium and Monoblepharidomycetes orders. In an aspect, the pest may be phylogenetically related to a Rozella spp.

Examples of fungi that infect microalgae cultures are members of the class Chytridiomycetes and members of the Chytridium spp. Chytrids are primitive fungi and are mostly saprophytic (degrading chitin and keratin). Some species are unicellular. As with other fungi, the cell wall in a chytrid is composed of chitin. Many chytrid species are aquatic (mostly found in fresh water). There are approximately 1,000 chytrid species, in 127 genera, distributed among 5 orders. Some chytrid species are parasitic and may infect plants, including microalgae.

Specific non-limiting examples of chyrids included in the present disclosure include Achiyogeton, Allochytridium, Allochytridium expandens, Allochytridium luteum, Allonmyces, Allomyces (subgenus), Allomyces attomyces, Allomyces catenoides, Allomyces reticulatus, Amoeboaphelidium protococcarum, Alphamycetaceae, Alphamyces, Alphamyces chaetiferum, Amphicypellus, Amphicypellus elegans, Anaeromyces, Anaeromyces elegans, Anaeromyces mucronatus, Angulomycetaceae, Angulomyces, Angulomyces argentinensis, Aquamycetaceae, Aquamyces, Aquamyces chlorogonii, Arnaudovia, Arnaudovia hyponeustonica, Asterophlyctis irregularis, Asterophlyctis sarcoptoides, Batrachochytrium, Batrachochytrium dendrobatidis, Blastocladia arborata, Blastocladia caduca, Blastocladia coronata, Blastocladia cristata, Blastocladia didyma, Blastocladia elegans, Blastocladia excelsa, Blastocladia filamentosa, Blastocladia fruticosa, Blastocladia fusiformis, Blastocladia globosa var. Minutissima, Blastocladia heterosporangia, Blastocladia mammilata, Blastocladia picaria, Blastocladia pileota, Blastocladia pusilla, Blastocladia sessilis, Blastocladia spiciformis, Blastocladiella, Blastocladiella anabaenae, Blastocladiella britannica, Blastocladiella colombiensis, Blastocladiella nova-zevlandiae, Blastocladiomycota, Blastocladiopsis elegans, Blyttiomyces bartsch, Blyttiomyces aureus booth, Bhyttiomyces conicus, Blyttiomyces exuviae, Blyttiomyces gregarum, Blyttiomyces harderi, Blyttiomyces laevis, Blyttiomyces lenis, Blyttiomyces rhizophlyctidis, Blyttiomyces spinosus, Blyttiomyces vaucheriae, Blyttiomyces verrucosus, Boothiomyces, Boothiomyces macroporosum, Caecomyces, Caecomyces communis, Caecomyces equi, Caecomyces sympodialis, Callimastix frontalis, Canteria, Canteria apophysata, Catenaria auxiliaris, Catenaria indica, Catenaria ramosa, Catenaria spinosa, Catenaria uncinata, Catenaria vermicola, Catenaria verrucosa, Catenochytridium hemicysti, Catenochytridium marinum, Catenochytridium oahuense, Catenophlyctis, Catenophlyctis peltata, Catenophlyctis variabilis, Catenophlyctis variabilis var. Olduvaiensis, Caulochytriaceae subramanium, Caulochytrium, Caulochytrium gloeosporii, Caulochytrium protostelioides, Caulochytrium protosteloides var. Vulgaris, Chytridiaceae, Chvtridiales, Chvtridiomycetes, Chytridiomycota, Chytridium, Chytridium adpressum, Chytridium aggregatum, Chytridium apophysatum, Chytridium brevipes, Chytridium cejpii, Chytridium chlorobotrris, Chytridium citriforme, Chytridium closterii, Chytridium codicola, Chytridium coleochaetes, Chytridium confervae, Chytridium corniculatum, Chytridium cresentum, Chytridium deltanum, Chytridium fusiforme, Chytridium gibbosum, Chytridium hemicysta, Chytridium horariumforme, Chytridium hiperparasiticum, Chytridium inflatum, Chytridium isthmiophilum, Chytridium kolianum, Chytridium lagenaria, Chytridium latipodium, Chytridium mallomonadis, Chytridium marylandicum, Chytridium mucronatum, Chytridium neopapillatum, Chytridium oedogonii, Chytridium ottariense, Chytridium parasiticum, Chytridium pilosum, Chytridium proliferum, Chytridium reniforme, Chytridium schenkii, Chytridium schenkii var. Dumontii, Chvytridium scherfelii, Chytridium sexuale, Chytridium sparrowii, Chytridium stellatum, Chytridium telmatoskenae, Chytridium turbinatum, Chytriomyces, Chytrionmyces angularis, Chytriomyces annulatus, Chytriomyces confervae, Chytriomyces cosmarii Chytriomyces elegans, Chytriomyces gilgaiensis, Chytriomyces heliozoicola, Chytriomyces hyalinus, Chytriomyces hyalinus var. Granulatus, Chytriomyces laevis, Chytriomyces macro-operculatus, Chytriomyces macro-operculatus var. Hirsutus, Chytriomyces mammilifer, Chytriomyces mortierellae, Chytriomyces multi-operculatus, Chytriomyces nagatoroensis, Chytrionmyces poculatus, Chytriomyces reticulatus, Chytriomyces reticulosporus, Chytriomyces rhizidiomycetis, Chytriomyces rotoruaensis, Chytriomyces suburceolatus, Chvtriomyces vallesiacus, Chytriomyces verrucosus, Chytriomyces willoughbyi, Cladochytriales, Cladochytriaceae, Cladochytrium aureum, Cladochytrium granulatum, Cladochytrium indicum, Cladochytrium novoguineense, Cladochytrium replicatum, Cladochytrium salsuginosum, Clydea, Clydea vesicula, Coelomomycetaceae, Coelomycidium, Coralloidiomyces, Coralloidiomyces digitatus, Cylindrochytrium endobioticum, Cystocladiella, Dangeardia appendiculata, Dangeardia echinulata, Dangeardia molesta, Dangeardia sporapiculata, Dangeardia sporapiculata var. Minor, Dangeardiana, Dangeardiana apiculata, Dangeardiana eudorinae, Dangeardiana leptorrhiza, Dangeardiana sporapiculata, Dictyomorpha, Dictyomorpha dioica, Dictyomorpha dioica var. Pythiensis, Diplochytridium, Diplochytridium aggregatum, Diplochytridium brevipes, Diplochytridium cejpii, Diplochyrridium chlorobotryis, Diplochytridium citriforme, Diplochytridium codicola, Diplochytridium gibbosum, Diplochytridium inflatum, Diplochytridium isthmiophilum, Diplochytridium kolianum, Diplochytridium lagenarium var. Japonense, Diplochytridium lagenarium, Diplochytridium mallomonadis, Diplochytridium mucronatum, Diplochytridium oedogonii, Diplochytridium schenkii, Diplochytridium scherffelii, Diplochytridium sexuale, Diplochytridium stellatum, Diplochytridium Turbinatum, Diplophlyctis asteroidea, Diplophlyctis buttermerensis, Diplophlyctis chitinophila, Diplophlyctis complicata, Diplophlyctis nephrochytrioides, Diplophlyclis sarcoptoides, Diplophlyctis serualis, Diplophlyctis versiformis, Endochytrium cystarurm, Endochytrium multiguttulatum, Entophlyctis, Entophlyctis apiculata, Entophlyctis bulligera, Entophlyctis bulligera var. Brevis, Entophlyctis caudiformis, Entophlyctis confervae-glomeratae, Entophlyctis crenata, Entophlyctis filamentosa, Entophlyctis helioformis, Entophlyctis lobata, Entophlyctis luteolus, Entophlyctis mammilliformis, Entophlyctis molesta, Entophlyctis obscura, Entophlyctis reticulospora, Entophlyctis rhizina, Entophlyclis sphaerioides, Entophlyclis texana, Entophlyctis variabilis, Entophlyctis variabilis, Entophlyctis vaucheriae, Entophlyctis willoughbyi, Gaertneriomyces, Gaertneriomyces semiglobiferus, Gaertneriomyces tenuis, Globomycetaceae, Globomyces, Globomyces pollinis-pini, Gonopodya terrestris, Gorgonomycetaceae, Gorgonomyces, Gorgonomyces haynaldii, Hapalopera, Hapalopera achnanthis, Hapalopera difficilis, Hapalopera fragilariae, Hapalopera melosirae, Hapalopera piriformis, Harpochytriaceae, Harpochytriales, Harpochytrium, Harpochytrium adpressum, Harpochytrium apiculatum, Harpochytrium botryococci, Harpochytrium hedenii, Harpochytrium hyalothecae, Harpochyrrium intermedium, Harpochytrium monae, Harpochytrium natrophilum, Harpochytrium ornithocephalum, Harpochytrium tenuissimum, Harpochytrium viride, Kappamyces, Kappamycetaceae, Kappamyces laurelensis, Karlingia, Karlingia aurantiaca, Karlingia exo-operculata, Karlingia expandens, Karlingia granulata, Karlingia lacustris, Karlingia lobata var. Microspora, Karlingia polonica, Karlingia spinosa, Karlingiomyces, Karlingiomyces laevis, Kochiomyces, Kochiomyces dichotomus, Krispiromyces, Krispiromyces discoides, Lacustromyces, Lacustromyces hiemalis, Lobulomycetales, Lobulomycetaceae, Lobulomyces, Lobulomyces angularis, Lobulomyces poculatus, Lyonomyces, Lyonomyces pyriformis, Macrochytrium botrydiella, Macrochytrium botrydioides var. Minutum, Maunachytrium, Maunachytrium keaense, Mesochytrium, Mesochytrium penetrans, Microallomyces, Microallomyces dendroideus, Micromyces furcata, Micromyces grandis, Micromycopsidaceae, Milleromyces, Milleromyces rhyniensis, Mitochytridium regale, Monoblepharidomycetes, Monoblepharidales, Monoblepharidaceae, Monoblepharella, Monoblepharis micrandra, Monoblepharis thalassinosus, Monophagus, Monophagus blackmanii, Monophagus bruhlii, Neocallimastigaceae, Neocallimastigales, Neocallimastigomycota, Neocallimastigomycetes, Neocallimastix, Neocallimastix frontalis, Neocallimastix hurleyensis, Neocallimastix jovonii, Neocallimastix patriciarum, Neocallimasti variabilis, Nephrochytrium bipes, Nephrochytrium buttermerense, Nephrochytrium complicatum, Nephrochytrium sexuale, Nowakowskiella crassa, Nowakowskiella delica, Nowakowskiella elegans, Nowakowskiella granulata, Nowakowskiella keratinophila, Nowakowskiella methistemichroma, Nowakowskiella moubasherana, Nowakowskiella multispora, Nowakowskiella multispora, Nowakowskiella pitcairnensis, Nowakowskiella profusa, Nowakowskiella profusaforma constricta, Nowakowskiella sculptura, Nowakowskiellaceae, Obelidium megarhizum, Oedogoniomycetaceae, Olpidium, Olpidium appendiculatum, Olpidium bornovanus, Olpidium brassicae, Olpidium cucurbitacearum, Olpidium entophlyctoides, Olpidium fulgens, Olpidium incognitum, Olpidium indicum, Olpidium indicum, Olpidium indum, Olpidium longum, Olpidium nematodae, Olpidium paradoxum, Olpidium poreferum, Olpidium pseudoeuglenae, Olpidium radicale, Olpidium rostriferum var. Indica, Olpidium sparrowii, Olpidium synchytrii, Olpidium vermicola, Olpidium virulentus, Olpidium wildemani, Olpidium zopfianus, Orpinomyces, Orpinomyces bovis, Orpinomyces intercalaris, Orpinomyces jovonii, Pateramycetaceae, Pateramyces, Pateramyces corrientinensis, Phlyctidium, Phlyctidium anatropum, Phlyctidium apophysatum, Phlyctidium brevipes var. Marinum, Phlyctidium bumilleriae, Phlyctidium globosum, Phlyctidium keratinophilum, Phlyctidium keratinophilum var. Savulescui, Phlyctidium marinum, Phlyctidium mycetophagum, Phlyctidium olla, Phlyctidium scenedesmi, Phlyctidium spinulosum, Phlyctidium tenue, Phlyctidium tubulatum, Phlyctochytrium acuminatum, Phlyctochytrium aestuarii, Phlyctochytrium africanum, Phlyctochytrium apophysatum, Phlyctochytrium arcticum, Phlyctochytrium aureliae, Phlyctochytrium californicum, Phlyctochytrium chandleri, Phlyctochytrium circulidentatum, Phlyctochytrium cystoferum, Phlyctochytrium dichotomum, Phlyctochytrium dissolutum, Phlyctochytrium furcatum, Phlyctochytrium hirsutum, Phlyctochytrium incrustans, Phlyctochytrium indicum, Phlyctochytrium irregulare, Phlyctochytrium kniepii, Phlyctochytrium lackeyi, Phlyctochytrium macrosporum, Phlyctochytrium mangrovii, Phlyctochytrium marilandicum, Phlyctochytrium megastomum, Phlyctochytrium mucosum, Phlyctochytrium multidentatum, Phlyctochytrium neuhausiae, Phlyctochytrium palustre, Phlyctochytrium parasitans, Phlyctochytrium peruvianum, Phlyctochytrium planicorne, Phlyctochytrium plurigibbosum, Phlyctochytrium powhatanense, Phlyctochytrium punctatum, Phlyctochytrium recurvastomum, Phlyctochytrium rhizopenicillium, Phlyctochytrium semiglobiferum, Phlyctochytrium spinosum, Phlyctochytrium variable, Phlyctochytrium vaucheriae, Phlyctochytrium verruculosum, Phlyctorhiza variabilis, Physodermataceae, Piromyces, Piromyces communis, Piromyces dumbonicus, Piromyces mae, Piromyces minutus, Piromyces rhizinflatus, Piromyces spiralis, Pleotrachelus, Pleotrachelus askaulos, Pleotrachelus bornovanus, Pleotrachelus brassicae, Pleotrachelus virulentus, Pleotrachelus wildemanni, Pleotrachelus zopfianus, Podochytrium chitinophilum, Podochytrium dentatum, Podochytrium ellerbeckense, Polyphagus asymmetricus, Polyphagus elegans, Polyphagus euglenae, Polyphagus hyponeustonica, Polyphagus serpentinus, Polyphagus starrii, Polyphlyctis, Polyphlyctis cystofera, Polyphlyctis unispina, Powellomyces, Powellomyces hirtus, Powellomyces variabilis, Pringsheimiella dioica, Protrudomycetaceae, Protrudomyces, Protrudomyces laterale, Pseudopileum, Pseudopileum unum, Rhizidium megastomum, Rhizidium phycophilum, Rhizidium renifore, Rhizidium tomiyamanum, Rhizoclosmatium hyalinum, Rhizophlyctidales, Rhizophlyctidaceae, Rhizophlyctis, Rhizophlyctis aurantiaca, Rhizophlyctis boninensis, Rhizophlyctis bonseyi, Rhizophlyctis columellae, Rhizophlyctis costatus, Rhizophlyctis fuscus, Rhizophlyctis hirsutus, Rhizophlyctis lovettii, Rhizophlyctis oceanis, Rhizophlyctis oceanis var. Floridaensis, Rhizophlyctis petersenii var. Appendiculata, Rhizophlyctis reynoldsii, Rhizophlyctis rosea, Rhizophlyctis serpentina, Rhizophlyctis sp., Rhizophlyctis tropicalis, Rhizophlyctis variabilis, Rhizophlyctis variabilis var. Burmaensis, Rhizophlictis willoughbyi, Rhizophydium, Rhizophydiales, Rhizophydiaceae, Rhizophydium achnanthis, Rhizophydium algavorum, Rhizophydium anatropum, Rhizophydium androdioctes, Rhizophydium angulosum, Rhizophydium annulatum, Rhizophydium aphanomycis, Rhizophydium aureum, Rhizophydium biporosum, Rhizophydium blastocladianum, Rhizophydium blyttiomycerum, Rhizophydium brevipes var. Marinum, Rhizophydium brooksianum, Rhizophydium bumilleriae, Rhizophydium capillaceum, Rhizophydium clavatum, Rhizophydium coleochaetes, Rhizophydium collapsum, Rhizophydium conchiforme, Rhizophydium condylosum, Rhizophydium contractophilum, Rhizophydium coralloidum, Rhizophydium dentatum, Rhizophydium difficile, Rhizophydium digitatum, Rhizophydium dubium, Rhizophydium echinocystoides, Rhizophydium ellipsoidium, Rhizophydium fragilariae, Rhizophydium fugax, Rhizophydium gonapodyanum, Rhizophydium hispidulosum, Rhizophydium karlingii, Rhizophydium lagenaria, Rhizophydium laterale, Rhizophydium lenelangeae, Rhizophydium littoreum, Rhizophydium macroporosum, Rhizophydium manoense, Rhizophydium melosirae, Rhizophydium mougeotiae, Rhizophydium nobile, Rhizophydium novae-zevlandiensis, Rhizophydium obpyriformis, Rhizophydium olla, Rhizophydium patellarium, Rhizophydium pedicellatum, Rhizophydium piriformis, Rhizophydium planktonicum, Rhizophydium poculiforme, Rhizophydium polystomum, Rhizophydium porosum, Rhizophydium proliferum, Rhizophydium punctatum, Rhizophydium rarotonganensis, Rhizophydium reflexum, Rhizophydium rhizinum, Rhizophydium rotundum, Rhizophydium scenedesmi, Rhizophydium sibyllinum, Rhizophydium signyense, Rhizophydium skujai, Rhizophydium sparrowii, Rhizophydium sphaerocarpum var. Rhizoclonii, Rhizophydium sphaerocarpum var. Spirogyrae, Rhizophydium sphaerotheca, Rhizophydium spinosum, Rhizophydium spinosum, Rhizophydium spinosum, Rhizophydium spinulosum, Rhizophydium squamosum, Rhizophydium stellatum, Rhizophydium tenue, Rhizophydium tetragenum, Rhizophydium tubulatum, Rhizophydium ubiquetum, Rhizophydium undatum, Rhizophydium undulatum, Rhizophydium urcelolatum, Rhizophydium venezuelensis, Rhizophydium venustum, Rozella, Rozella blastocladiae, Rozella coleochaetis, Rozella diplophlyctidis, Rozella itersoniliae, Rozella longicollis, Rozella longisporangia, Rozella parva, Ruminomyces, Ruminomyces elegans, Scherffeliomyces appendiculatus, Scherffeliomyces leptorrhizus, Scherffeliomycopsis, Scherffeliomycopsis coleochaetis, Septochytriaceae, Septochytrium willoughbyi, Septosperma, Septosperma anomalum, Septosperma irregulare, Septosperma multifirma, Septosperma rhizophidii, Septosperma spinosa, Siphonaria variabilis, Solutoparies, Sorochytriaceae, Sorochytrium, Sorochytrium milnesiophthora, Sparrowia, Sparrowia parasitica, Sparrowia subcruciformis, Sparrowmyces, Sparrowmyces sparrowii, Sphaerita dinobryi, Spizellomyces, Spizellomyces acuminatus, Spizellomyces dolichospermus, Spizellomyces kniepii, Spizellomyces lactosolyticus, Spizellomyces palustris, Spizellomyces plurigibbosus, Spizellomyces pseudodichotomus, Spizellomyces punctatus, Spizellomycetaceae, Spizellomycetales, Sporophlyctidium neustonicum, Synchytrium, Terramycetaceae, Terramyces, Terramyces subangulosum, Thallasochytrium, Thallasochytrrium gracillariopsidis, Triparticalcar, Triparticalcar arcticum, Urceomyces, Urceomyces sphaerocarpum, Urophlyctaceae, Zygorhizidium affluens, Zygorhizidium asterionellae, Zygorhizidium chlorophycidis, Zygorhizidium cystogenum, Zygorhizidium melosirae, Zygorhizidium planktonicum, Zygorhizidium planktonicum, Zygorhizidium vaucheriae, Zygorhizidium venustum. See also, Barr, D. J. S. “An outline for the reclassification of the Chytridiales, and for a new order, the Spizellomycetales,” Canadian Journal of Botany, 58: 2380-2394 (1980); Barr, D. J. S., “In: Handbook of Protoctista,” Eds. L. Margulis, J. O. Corliss, M. Melkonian, and D. J. Chapman, Jones & Bartlett Publishers, Boston, Mass. (Abbreviation HP), Phylum Chytridiomycota, pp. 454-466 (1990); Batko, A., Zarys Hydromikologii. Panstwowe Wydawnictwo Naukowe, Warsaw, Poland (Abbreviation ZH) (1975); Index of fungi, C.A.B. International, Wallingford, United Kingdom (Abbreviation IF) vols. 3-6 (1960-1995); Hibbett, D. S. et al., “A higher-level phylogenetic classification of the Fungi,” Mycological Research, 111:509-547 (2007); James, T. Y., et al., “Molecular phylogenetics of the Chytridiomycota supports the utility of ultrastructural data in chytrid systematics,” Canadian Journal of Botany, 78:336-350 (2000); James, T. Y. et al., “Reconstructing the early evolution of Fungi using a six-gene phylogeny,” Nature 443:818-822 (2006); James, T. Y. et al., “A molecular phylogeny of the flagellated fungi (Chytridiomycota) and description of a new phylum (Blastocladiomycota),” Mycologia, 98:860-871 (2006); Karling, J. S., “Chytridiomycetarum Iconographia (Abbreviation CI),” Lubrect & Cramer, Monticello, N.Y., (1977); Letcher, P. M. et al., “Ultrastructural and molecular delineation of the Chytridiaceae (Chytridiales),” Canadian Journal of Botany, 82:1561-1573 (2005); Letcher, P. M. et al., “Ultrastructural and molecular phylogenetic delineation of a new order, the Rhizophydiales (Chytridiomycota),” Mycological Research, 110:898-915 (2006); Letcher, P. M. et al., “Rhizophyctidales—a new order in Chtridiomycota,” Mycological Research, 112:1031-1048 (2008); Li et al., “The phyogenetic relationships of the anaerobic chytridiomycetous gut fungi (Neocallimasticaceae) and the Chytridiomycota. II. Cladistic analysis of structural data and description of Neocallimasticales ord. nov.,” Canadian Journal of Botany, 71:393-407 (1993); Mozley-Standridge, S. E. et al. “Cladochytriales, a new order in Chytridiomycota,” Mycological Research, 113:498-507 (2009); Simmons, D. R. et al. “Lobulomycetales, a new order in the Chytridiomycota,” Mycological Research, 113:450-460 (2009); Sparrow, F. K., “Aquatic Phycomycetes,” 2nd rev. ed., University of Michigan Press, Ann Arbor, Mich. (1960); and Sparrow, F. K., “Chytridiomycetes, Hyphochytridiomycetes. In: The Fungi,” IVB Eds., G. C. Ainsworth, F. K. Sparrow and A. S. Sussman, Academic Press, New York, pp. 85-110 (1973), each of which are hereby incorporated by reference in their entireties.

In another aspect, a pest may be a protozoan. In an aspect a protozoan may be an amoeba. In another aspect a protozoan may be Vannella danica. In a further aspect, a protozoan may be a ciliate. In an aspect a ciliate may be Cyclidium glaucoma or Euplotes minuta.

In further aspect of the invention, a pest may be a bacterium. In an aspect the bacterium may be member of the Halomonadaceae family. In an aspect, a pest may be a species of the genus Halamonas. In an aspect, pest may be Halomonas campisalis.

In an aspect, a pest may be a member of the rotifer phylum. In a further aspect a rotifer may be rotifer in the family Brachionidae. In an aspect the Brachionidae may be Brachionus plicatilis.

In addition to fungal pests, a number of other pests according to the present disclosure have been sequence identified, and designated as pests of algae. These include eukaryotic species of amoeba, ciliates, rotifers as well as prokaryotes such as Halomonas.

Microalgae of the present disclosure include members of the chlorophyta division of the protist kingdom. Microalgae of the present disclosure include members of the Chlamydomonas sp. In one aspect the microalgae of the present disclosure is Chlamydomonas reinhardtii (C. reinhardtii). In another aspect of the present disclosure, the C. reinhardtii may be genetically engineered. In yet another aspect, the member of the chlorophyta may be a Scenedesmus sp. In another aspect the chlorophyte may be a member of the Chlorella sp. In another aspect, the chlorophyte may be a member of the Desmodesmus sp. The chlorophytes of the present disclosure may be genetically engineered.

Common non-limiting examples of non-vascular photosynthetic organisms (NVPO) that can be used with the methods disclosed herein are members of one of the following divisions: chlorophyta, cyanophyta (cyanobacteria), and heterokontophyta, bacillariophyta, chrysophyta and haptophyta. In some instances, the microalgae are, for example, an organism classified as prochlorophyta, rhodophyta, tribophyta, glaucophyta, chlorarachniophytes, euglenophyta, euglenoids, cryptophyta, cryptomonads, dinophyta, dinoflagellata, pyrmnesiophyta, bacillariophyta, xanthophyta, eustigmatophyta, raphidophyta, phaeophyta, and phytoplankton.

Specific non-limiting examples of chlorophytes include Ankistrodesmus, Botryococcus, Chlorella, Chlorococcum, Dunaliella, Monoraphidium, Oocystis, Scenedesmus, Desmodesmus, and Tetraselmis. In one aspect, the chlorophytes can be Chlorella or Dunaliella. Specific non-limiting examples of cyanophytes include Oscillatoria and Synechococcus. Specific example of chrysophytes includes Boekelovia. Specific non-limiting examples of haptophytes include Isochrysis and Pleurochrysis. Specific non-limiting examples of bacillariophytes include the genera Amphipleura, Amphora, Chaetoceros, Cyclotella, Cymbella, Fragilaria, Hantzschia, Navicula, Nitzschia, Phaeodactylum, and Thalassiosira.

In certain aspects, the NVPO used with the methods of the disclosure are members of one of the following genera: Nannochloropsis, Chlorella, Dunaliella, Scenedesmus, Desmodesmus, Selenastrum, Oscillatoria, Phormidium, Spirulina, Nostoc, Amphora, and Ochromonas.

Non-limiting examples of NVPO species that can be used with the methods of the present disclosure include: Achnanthes orientalis, Agmenellum spp., Amphiprora hyaline, Amphora coffeiformis, Amphora coffeiformis var. linea, Amphora coffeiformis var. punctata, Amphora coffeiformis var. taylori, Amphora coffeiformis var. tenuis, Amphora delicatissima, Amphora delicatissima var. capitata, Amphora sp., Anabaena, Ankistrodesmus, Ankistrodesmus falcatus, Boekelovia hooglandii, Borodinella sp., Botryococcus braunii, Botryococcus sudeticus, Bracteococcus minor, Bracteococcus medionucleatus, Carteria, Chaetoceros gracilis, Chaetoceros muelleri, Chaetoceros muelleri var. subsalsum, Chaetoceros sp., Chlamydomas perigranulata, Chlorella anitrata, Chlorella antarctica, Chlorella aureoviridis, Chlorella Candida, Chlorella capsulate, Chlorella desiccate, Chlorella ellipsoidea, Chlorella emersonii, Chlorella fusca, Chlorella fusca var. vacuolate, Chlorella glucorropha, Chlorella infusionum, Chlorella infusionum var. actophila, Chlorella infusionum var. auxenophila, Chlorella kessleri, Chlorella lobophora, Chlorella luteoviridis, Chlorella luteoviridis var. aureoviridis, Chlorella luteoviridis var. lutescens, Chlorella miniata, Chlorella minutissima, Chlorella mutabilis, Chlorella nocturna, Chlorella ovalis, Chlorella parva, Chlorella photophila, Chlorella pringsheimii, Chlorella protothecoides, Chlorella protothecoides var. acidicola, Chlorella regularis, Chlorella regularis var. minima, Chlorella regularis var. umbricata, Chlorella reisiglii, Chlorella saccharophila, Chlorella saccharophila var. ellipsoidea, Chlorella salina, Chlorella simplex, Chlorella sorokiniana, Chlorella sp., Chlorella sphaerica, Chlorella stigmatophora, Chlorella vanniellii, Chlorella vulgaris, Chlorella vulgaris fo. tertia, Chlorella vulgaris var. autotrophica, Chlorella vulgaris var. viridis, Chlorella vulgaris var. vulgaris, Chlorella vulgaris var. vulgaris fo. tertia, Chlorella vulgaris var. vulgaris fo. viridis, Chlorella xanthella, Chlorella zofingiensis, Chlorella trebouxioides, Chlorella vulgaris, Chlorococcum infusionum, Chlorococcum sp., Chlorogonium, Chroomonas sp., Chrysosphaera sp., Cricosphaera sp., Crypthecodinium cohnii, Cryptomonas sp., Cyclotella cryptica, Cyclotella meneghiniana, Cyclotella sp., Dunaliella sp., Dunaliella bardawil, Dunaliella bioculata, Dunaliella granulate, Dunaliella maritime, Dunaliella minuta, Dunaliella parva, Dunaliella peircei, Dunaliella primolecta, Dunaliella salina, Dunaliella terricola, Dunaliella tertiolecta, Dunaliella viridis, Dunaliella tertiolecta, Eremosphaera viridis, Eremosphaera sp., Ellipsoidon sp., Euglena spp., Franceia sp., Fragilaria crotonensis, Fragilaria sp., Gleocapsa sp., Gloeothamnion sp., Haematococcus pluvialis, Hymenomonas sp., Isochrysis aff. galbana, Isochrysis galbana, Lepocinclis, Micractinium, Micractinium, Monoraphidium minutum, Monoraphidium sp., Nannochloris sp., Nannochloropsis salina, Nannochloropsis sp., Navicula acceptata, Navicula biskanterae, Navicula pseudotenelloides, Navicula pelliculosa, Navicula saprophila, Navicula sp., Nephrochloris sp., Nephroselmis sp., Nitschia communis, Nitzschia alexandrine, Nitzschia closterium, Nitzschia communis, Nitzschia dissipata, Nitzschia frustulum, Nitzschia hantzschiana, Nitzschia inconspicua, Nitzschia intermedia, Nitzschia microcephala, Nitzschia pusilla, Nitzschia pusilla elliptica, Nitzschia pusilla monoensis, Nitzschia quadrangular, Nitzschia sp., Ochromonas sp., Oocystis parva, Oocystis pusilla, Oocystis sp., Oscillatoria limnetica, Oscillatoria sp., Oscillatoria subbrevis, Parachlorella kessleri, Pascheria acidophila, Pavlova sp., Phaeodactylum tricomutum, Phagus, Phormidium, Platymonas sp., Pleurochrysis camerae, Pleurochrysis dentate, Pleurochrysis sp., Prototheca wickerhamii, Prototheca stagnora, Prototheca portoricensis, Prototheca moriformis, Prototheca zopfii, Pseudochlorella aquatica, Pyramimonas sp., Pyrobotrys, Rhodococcus opacus, Sarcinoid chrysophyte, Scenedesmus armatus, Schizochytrium, Spirogyra, Spirulina platensis, Stichococcus sp., Synechococcus sp., Synechocystisf, Tagetes erecta, Tagetes patula, Tetraedron, Tetraselmis sp., Tetraselmis suecica, Thalassiosira weissflogii, and Viridiella fridericiana.

Other examples of non-vascular photosynthetic organisms are C. reinhardtii, D. salina, D. tertiolecta, S. dimorphus, or H. pluvialis. The organism can be a member of the genus Chlamydomonas, Dunaliella, Scenedesmus, Desmodesmus or Hematococcus, for example C. reinhardtii, D. salina, D. tertiolecta, S. dimorphus or H. pluvialis, although members of other genera may be used.

One organism that can be cultured as described herein is a commonly used species C. reinhardtii. Cells of this species are haploid, and can grow on a simple medium of inorganic salts, using photosynthesis to provide energy. This organism can also grow in total darkness if acetate is provided as a carbon source. C. reinhardtii can be readily grown at room temperature under standard fluorescent lights. In addition, the cells can be synchronized by placing them on a light-dark cycle. Other methods of culturing C. reinhardtii cells are known to one of skill in the art. Methods for culturing organisms of the present disclosure are known in the art, for example, in Vonshak, A. Spirulina Platensis (Arthrospira): Physiology, Cell-Biology And Biotechnology. 1997. CRC Press, Andersen, A. Algal Culturing Techniques. 2005. Elsevier Academic Press, Chen et al. (2011) “Cultivation, photobioreactor design and harvesting of microalgae for biodiesel production: A critical review,” Bioresource Technology 102:71-81, Rodolfi et al., “Microalgae for oil: Strain selection, induction of lipid synthesis and outdoor mass cultivation in a low-cost photobioreactor”, Biotechnology and Bioengineering 102:100-112 (2009), and Ugwu et al., “Photobioreactors for mass cultivation of algae,” Bioresource Technology 99:4021-4028 (2008), each of which is hereby incorporated by reference in their entirety.

In another aspect, microalgae of the present disclosure include members of the phyla heterokontophyta. In an aspect, a microalga of the phyla heterokontophyta may be member of the genus Nannochloropsis. In another aspect, a Nannochloropsis may be genetically engineered. In an aspect, a microalga of the present disclosure may be a microalgae of the chorophyta division of the protist kingdom.

In another aspect, microalgae of the present disclosure may be a cyanobacterium. In an aspect, a cyanobacterium may be a member of the genus Spirulina, or of the genus Leptolyngbya or the genus Nostoc. In another aspect the microalgae may be a Desmid.

In an aspect, microalgae of the present disclosure may be genetically engineered. In an aspect the microalgae of the present disclosure may be genetically engineered according to the methods of International Patent Application No. PCT/US2010/048828, published as International Paten Publication WO 2011/034863, or according to the methods provided in International Patent Application No. PCT/US2010/048666, published as International Publication No. WO 2011/034823, both of which are hereby incorporated by reference in their entireties.

In an aspect of the present disclosure, a microalga is grown in a liquid system. In one aspect, the microalgae are inoculated into a liquid as a single species of microalgae. In one aspect, the microalgae may be a transformed microalgae having one or more exogenous DNA sequences. In a different aspect, the microalgae may have sequences that are endogenous DNA sequences in a recombinant construct. In another aspect, the sequences may be exogenous DNA sequences in a recombinant construct.

In another aspect, a single species of microalgae may be a population of microalgae. In one aspect, a population of microalgae may be transformed with one or more DNA constructs. In an aspect, a population of microalgae may be a mixture of a single species having one or more DNA constructs.

In an aspect, the liquid system may have more than one species of microalgae. In one aspect, the liquid system may have two species of microalgae. In another aspect, the liquid system may have three species of microalgae. In still another aspect, the liquid system may have between 4 to 6 species, 6 to 8 species or 8 to 10 species of microalgae. In yet another aspect, one or more of the more than one species of microalgae in the liquid system may be genetically transformed. The one or more genetically transformed species may be contain the same genetic transformation or they may contain different transformations.

In an aspect of the present disclosure, the liquid system may have two or more species of microalgae selected from the genus Spirulina. In another aspect, the liquid system may have two or more species of microalgae selected from the genus Scenedesmus. In a further aspect, the liquid system may have two or more species of microalgae selected from the genus Desmodesmus. In an aspect, the liquid system may have two or more species of microalgae selected from the genus Leptolyngbya. In an aspect, the liquid system may have two or more species of microalgae selected from the genus Nostoc. In an aspect, the two or more species of microalgae may be transformed.

In an aspect, the liquid system may have two species of microalgae, one species selected from one genus and a second species selected from a second genus. In an aspect, the first genus may be Spirulina and the second genus may be Scenedesmus. In an aspect, the first genus may be Spirulina and the second genus may be Desmodesmus. In an aspect, the first genus may be Spirulina and the second genus may be Leptolyngbya. In an aspect, the first genus may be Scenedesmus and the second genus may be Leptolyngbya. In yet another aspect of the present disclosure, the first genus may be Leptolyngbya and the second genus may be Desmodesmus.

In a further aspect, the liquid system may have three species of microalgae selected from a genus. In an aspect, the liquid system may have three species of microalgae selected from the genus Spirulina. In another aspect, the liquid system may have three species of microalgae selected from the genus Scenedesmus. In a further aspect, the liquid system may have three species of microalgae selected from the genus Desmodesmus. In an aspect, the liquid system may have three species of microalgae selected from the genus Leptolyngbya.

In an aspect, the three species of microalgae may be genetically transformed. In an aspect, the liquid system may have three species of microalgae, one species selected from one genus, a second species selected from a second genus and a third species selected from a third genus. In an aspect, the first genus may be Spirulina, the second genus may be Scenedesmus, and the third genus may be Leptolyngbya. In an aspect, the first genus may be Spirulina, the second genus may be Desmodesmus, and the third genus may be Leptolyngbya. In an aspect, the three species of microalgae may be transformed. In an aspect, the liquid system may comprise 4, 5, 6, 7, 8, 9, 10 or more combinations of species of microalgae selected from the genera of Spirulina, Scenedesmus, Desmodesmus and Leptolyngbya.

A liquid of the liquid system of the present disclosure may be a defined or undefined media. In one aspect, the liquid may include untreated water. In an aspect, the untreated water may be water obtainable from a natural source such as a river, lake, aquifer, ocean or a pond. In another aspect, the liquid may be brackish water having an osmolarity between 0.5 and 30 grams of salt per liter. In yet another aspect, the liquid may be salt water. In an aspect, the water may be recycled water obtainable from a sewage or waste water treatment plant, or waste water from an industrial process such as power production and the like. In an aspect of the present disclosure, the untreated water may be aquifer water. In a further aspect, the untreated water may be aquifer water that is not suitable for agriculture. In yet another aspect, the aquifer water may be aquifer water with an elevated total dissolved solids (TDS).

A liquid of the liquid system may be supplemented with nutrients that benefit the growth of the microalgae. In one aspect, the liquid may be supplemented with CO₂ to enhance the growth of the microalgae. In an aspect the CO₂ may be introduced into the liquid system by bubbling with air or CO₂. Bubbling with CO₂ can be, for example, at 1% to 5% CO₂. CO₂ can be delivered to the liquid system as described herein, for example, by bubbling in CO₂ from under the surface of the liquid containing the microalgae. Also, sparges can be used to inject CO₂ into the liquid. Spargers are, for example, porous disc or tube assemblies that are also referred to as bubblers, carbonators, aerators, porous stones and diffusers. In an aspect the CO₂ may be introduced into the liquid system as a liquid.

In an aspect, the liquid may be supplemented with CO₂ to increase the concentration of CO₂ in the liquid to 20 parts-per-million (ppm), or more. In another aspect, the liquid may be supplemented with CO₂ to increase the concentration of CO₂ in the liquid to 25 ppm, or more. In yet another aspect, the liquid may be supplemented with CO₂ to increase the concentration of CO₂ in the liquid to 30 ppm, or more. In another aspect, the liquid of the liquid system may be supplemented with CO₂ to increase the concentration of CO₂ in the liquid to 35 ppm, or more.

In an aspect, a liquid system may be supplemented with CO₂ to maintain the pH of the liquid system. When the microalgae photosynthesize they drive the pH of a liquid system up. If at any time the pH surpasses an upper limit of a threshold, CO₂ is added to the pond until the pH decreases to the specified range. In an aspect, a liquid system inoculated with green algae is supplemented with CO₂ to maintain a pH of 8.8 to 9.2. In an aspect the liquid system is inoculated with chlorophyta and maintained at a pH of 8.8 to 9.2. In an aspect the liquid system is inoculated with Scenedesmus and maintained at a pH of 8.8 to 9.2. In an aspect, the liquid system may be inoculated with Scenedesmus dimorphous and maintained at a pH of 8.8 to 9.2. In another aspect, a liquid system inoculated with a blue-green alga of the phylum Cyanophyta and supplemented with CO₂ to maintain a pH of 9.8 to 10.2. In another aspect, a liquid system inoculated with a blue-green alga of the genus Spirulina and supplemented with CO₂ to maintain a pH of 9.8 to 10.2. In an aspect, a liquid system inoculated with a blue-green alga of the species Spirulina platensis and supplemented with CO₂ to maintain a pH of 9.8 to 10.2.

In an aspect of the present disclosure, the pH of a liquid system is monitored as a proxy for the amount of CO₂ available for photosynthesis. In an aspect, a liquid system being provided with CO₂ may have a pH defined as an upper limit. When a liquid system being provided CO₂ reaches an upper limit, CO₂ is provided to lower the pH. In an aspect, the upper pH limit may be 9.2. In another aspect, the upper pH limit may be 9.4. In another aspect, upper limit for pH may be set at 9.4. In a further aspect, the upper limit for pH may be set at 9.6. In another aspect, the upper limit for pH may be set at 9.8. In still another aspect, the upper limit for pH may be set at 10.2, 10.4 or 10.6.

In an aspect of the present disclosure, a liquid system being provided with CO₂ may have a pH defined as a lower limit. In an aspect, CO₂ supply is terminated to the liquid system when the pH drops below a pre-defined threshold in order to raise the pH. In an aspect, the threshold may be a pH of 8.8. In another aspect, the threshold may be 9.8. In yet another aspect, the threshold may be 9.0. In an aspect the threshold may be 9.2. In a further aspect, the threshold may be 9.4. In yet another aspect, the threshold may be 9.6.

It is understood that the present disclosure provides for the addition of CO₂ to maintain a pH within a range with the threshold and limit pH values being set accordingly. It is further understood that different species of microalgae have different preferred pH ranges for optimal growth. The threshold and limit pH values may be determined experimentally to maximize the photosynthesis and growth of microalgae in a liquid culture system. In an aspect of the present disclosure the pH range may be maintained between 8.8 and 9.2. In another aspect, the pH range may be maintained between 8.8 and 9.4. In a further aspect, the pH may be maintained between 8.8 and 9.6. In an aspect, the pH may be maintained between 8.8 and 9.8. In an aspect, the pH range may be between 9.8 and 10.2. In another aspect, the pH may be between 9.6 and 10.2. In an aspect, the pH may be between 9.4 and 10.2.

In an aspect of the present disclosure, the liquid system may be supplemented with CO₂ to provide a concentration of CO₂ in the liquid to 20 parts-per-million (ppm), or more. In another aspect, the liquid may be supplemented with CO₂ to increase the concentration of CO₂ in the liquid to 25 ppm, or more. In yet another aspect, the liquid may be supplemented with CO₂ to increase the concentration of CO₂ in the liquid to 30 ppm, or more. In another aspect, the liquid of the liquid system may be supplemented with CO₂ to increase the concentration of CO₂ in the liquid to 35 ppm, or more.

The present disclosure also provides for the supplementation of the liquid system with nutrients. Nutrients that can be used in the systems described herein, or known in the art, include, for example, nitrogen, phosphorus, and trace metals. In an aspect, nitrogen may supplemented in the form of ammonia or ammonium. In one aspect ammonium is provided as ammonium sulfate or ammonium chloride. In another aspect, the nitrogen supplement may be provided as urea. In an aspect, the supplemental nitrogen may be provided as nitrate or nitric acid. In yet another aspect, the supplemental nitrogen may be provided as a mixture, for example as a mixture of urea and ammonium nitrate, also known as URAN. In an aspect, the nitrogen may be provided as potassium nitrate (KNO3). In an aspect, the nitrogen may be provided as sodium nitrate (NaNO3).

A liquid system of the present disclosure may be supplemented with trace metals. Supplements of trace metals may include salts of iron (Fe), magnesium (Mg), potassium (K), calcium (Ca), cobalt (Co), copper (Cu), manganese (Mn), molybdenum (Mo), zinc (Zn), vanadium (V) or boron (B). In an aspect the trace metal may be supplied in the form of a nitrate (NO₃⁻) or ammonium (NH₄⁺) salt. In an aspect, potassium may be added as potassium chloride or potassium sulfate. In another aspect, potassium may be added to the liquid system as potassium nitrate. The nutrients can come, for example, in a solid form or in a liquid form. If the nutrients are in a solid form they can be mixed with, for example, fresh or salt water prior to being delivered to the liquid system containing the organism, or prior to being delivered to a culture system. In an aspect, a nutrient is applied in a manner that minimizes the potential of osmotic stress to the cells. In an aspect, nutrient additions are done over an extended period of time. In a further aspect, the nutrients may be diluted prior to being applied to a pond.

A liquid system of the present disclosure may be maintained at a preferred pH depending on the microalgae. In an aspect, a neutral pH may be maintained. In one aspect, the pH may be maintained between a pH of 6.5 and 7.5. In another aspect, an alkaline pH may be maintained, for example, a pH of 10. In an aspect, an alkaline pH in the range of 8.0 to 11.0 may be maintained. In yet another aspect, the pH of the liquid system may be acidic, for example, a pH of 6.0. In another aspect, an acidic pH of the liquid system may be a pH from about 4.0 to about 6.5.

Microalgae can be cultured in defined media known in the art, such as min-70, M-medium, or Tris acetate phosphate (TAP) medium. Organisms can be grown on a defined minimal medium (for example, high salt medium (HSM), modified artificial sea water medium (MASM), or F/2 medium) with light as the sole energy source. In other instances, the organism can be grown in a medium (for example, TAP medium), and supplemented with an organic carbon source. In an aspect, cyanobacteria may be grown in a medium (for example, BG-11)

Organisms, such as microalgae, can grow naturally in fresh water or marine water. Culture media for freshwater microalgae can be, for example, synthetic media, enriched media, soil water media, and solidified media, such as agar. Various culture media have been developed and used for the isolation and cultivation of fresh water microalgae and are described in Watanabe, M. W. (2005). Freshwater Culture Media. In R. A. Andersen (Ed.), Algal Culturing Techniques (pp. 13-20). Elsevier Academic Press. Culture media for marine microalgae can be, for example, artificial seawater media or natural seawater media. Guidelines for the preparation of media are described in Harrison, P. J. and Berges, J. A. (2005). Marine Culture Media. In R. A. Andersen (Ed.), Algal Culturing Techniques (pp. 21-33). Elsevier Academic Press.

In an aspect, Desmid (e.g., Scenedesmus and Desmodesmus) media may be: 1.929 g/L sodium bicarbonate, 0.1 g/L urea, 2.3730 g/L sodium sulfate, 0.52 g/L sodium chloride, 0.298 g/L potassium chloride, 0.365 g/L magnesium sulfate, 0.084 g/L sodium fluoride, 0.035 mL/L 75% phosphoric acid, 0.018 g/L Librel® Fe-Lo (BASF), 0.3 mL/L 20× iron stock solution (20× iron stock solution: 1 g/L sodium ethylenediaminetetraacetic acid (EDTA) and 3.88 g/L iron chloride) and 0.06 mL/L 100× trace metal stock solution (100× trace metal stock solution: 1 g/L sodium ethylenediaminetetraacetic acid, 7.2 g/L manganese chloride, 2.09 g/L zinc chloride, 1.26 g/L sodium molybdate, and 0.4 g/L cobalt chloride. In an aspect, Spirulina media may be: 3.675 g/L sodium bicarbonate, 4.766 g/L sodium sulfate, 1.09 g/L sodium chloride, 0.49 g/L potassium chloride, 0.518 g/L magnesium sulfate, 0.146 g/L sodium fluoride, 0.306 mL/L 67% nitric acid, 0.0173 mL/L 75% phosphoric acid, 0.018 g/L Librel Fe-Lo, 0.3 mL/L 20× iron stock solution, and 0.06 mL/L 100× trace metal stock solution. In an aspect, Nannochloropsis media may be: 3.675 g/L sodium bicarbonate, 4.766 g/L sodium sulfate, 1.09 g/L sodium chloride, 1.09 g/L potassium chloride, 3.018 g/L magnesium sulfate, 0.146 g/L sodium fluoride, 0.3 g/L calcium chloride, 0.293 mL/L 67% nitric acid, 0.0173 mL/L 75% phosphoric acid, 50 mL 20× iron stock solution, and 10 mL/L 100× trace metal stock solution.

Organisms may be grown in outdoor open water, such as ponds, the ocean, seas, rivers, waterbeds, marshes, shallow pools, lakes, aqueducts, and reservoirs. When grown in water, the organism can be contained in a halo-like object comprised of lego-like particles. The halo-like object encircles the organism and allows it to retain nutrients from the water beneath while keeping it in open sunlight.

In accordance with the present disclosure, the microalgae can be grown in open and/or closed systems that can vary in volume over a wide range. Closed systems can include reservoir structures, such as ponds, troughs, or tubes, which are protected from the external environment and have controlled temperatures, atmospheres, and other conditions. Closed systems may obtain the light required for photosynthesis artificially or naturally. For some embodiments, the microalgae may be grown in the absence of light and/or in the presence of an organic carbon source. Optionally, microalgae growth reservoirs can include a carbon dioxide source and a circulating mechanism configured to circulate microalgae within the microalgae growth reservoirs. Other examples of closed growth environments or reservoirs include closed bioreactors.

In an open microalgae culture system, at least one aspect of the liquid system is open to the environment. An open liquid system may be provided with light for photosynthesis artificially or naturally. For some embodiments, the microalgae may be grown in the absence of light and/or in the presence of an organic carbon source. In large open systems, natural light is often used. An open system allows the free exchange of nutrients and products, for example oxygen and carbon dioxide with the air. One way to achieve large surface growth areas is in large ponds or in a captive marine environment. In some aspects, a raceway pond can be used as a microalgae growth reservoir in which microalgae are grown in shallow circulating ponds with constant movement around the raceway and constant extraction or skimming off of mature microalgae. In other aspects, microalgae are grown in non circulating pools.

In both open and closed systems, microalgae cultures can become host to other biological organisms that can decrease the production of microalgae by competing for nutrients. Pest organisms are a significant problem for the efficient production of commercial products of interest by microalgae. In other cases, infection of a microalgae culture can completely destroy production either by competition or by parasitism. Non-limiting examples of pests are bacteria and fungi.

In some instances, organisms can be grown in containers wherein each container comprises one or two organisms, or a plurality of organisms. The containers can be configured to float on water. For example, a container can be filled by a combination of air and water to make the container and the organism(s) in it buoyant. An organism that is adapted to grow in fresh water can thus be grown in salt water (i.e., the ocean) and vice versa. This mechanism allows for automatic death of the organism if there is any damage to the container.

Culturing techniques for microalgae include those described, for example, in Freshwater Culture Media. In R. A. Andersen (Ed.), Algal Culturing Techniques. Elsevier Academic Press, herein incorporated by reference in its entirety.

Because photosynthetic organisms, for example, microalgae, require sunlight, CO₂ and water for growth, they can be cultivated in, for example, open ponds and lakes. However, these open systems are more vulnerable to contamination with a pest than a closed system. One challenge with using an open system is that the organism of interest may not grow as quickly as a pest. This becomes a problem when a pest invades the liquid environment in which the organism of interest is growing, and the invading pest has a faster growth rate and takes over the system.

In addition, in open systems there is less control over water temperature, CO₂ concentration, and lighting conditions. A growing season of the organism is largely dependent on location and, aside from tropical areas, is limited to the warmer months of the year. In addition, in an open system, the number of different organisms that can be grown is limited to those that are able to survive in the chosen location. An open system, however, is cheaper to set up and/or maintain than a closed system. Open systems are generally unable to control variables such as temperature, humidity and light. These variables will vary in accordance with the climate in which they are situated. Thus, one of ordinary skill in the art would understand that selection of the organism for growth in an open system may be determined by the local climate of the open system. In an aspect, temperatures over a season in an open system may range from below freezing to above 110° F.

Another approach to growing an organism is to use a semi-closed system, such as covering the pond or pool with a structure, for example, a “greenhouse-type” structure. While this can result in a smaller system, it addresses many of the problems associated with an open system. The advantages of a semi-closed system are that it can allow for a greater number of different organisms to be grown, it can allow for an organism to be dominant over an invading organism by allowing the organism of interest to out compete the invading organism for nutrients required for its growth, and it can extend the growing season for the organism. For example, if the system is heated, the organism can grow year round.

A variation of the pond system is an artificial pond, for example, a raceway pond. In raceway ponds, the organism, water, and nutrients circulate around a “racetrack.” Paddlewheels provide constant motion to the liquid in the racetrack, allowing for the organism to be circulated back to the surface of the liquid at a chosen frequency. Paddlewheels also provide a source of agitation and oxygenate the system. These raceway ponds can be enclosed, for example, in a building or a greenhouse, or can be located outdoors. It will be apparent to one skilled in the art, that other designs of artificial ponds may be used in addition to raceway ponds and that other means of motivating liquid other than paddlewheels, such as pumps, may also be used.

Some of the organisms which may be grown in the liquid systems described herein are halophilic. For example, D. salina can grow in ocean water and salt lakes (salinity from 30-300 parts per thousand) and high salinity media (e.g., artificial seawater medium, seawater nutrient agar, brackish water medium, seawater medium, etc.). In one embodiment, D. salina may be grown in a media that is 3.0 molar salt. In another embodiment, D. salina may be grown in a media that is 3.2 molar salt. In a further aspect, D. salina may be grown in a media that is 3.4 molar salt. In other aspects, the molarity of the media for growing D. salina may be 3.6 molar. In yet another aspect, D. salina may be grown in a media that is 3.8 molar salt. In a further aspect, the D. salina growth media may be 4.0 molar salt. In an aspect, the salt may be sodium chloride. In another aspect, the media may be ocean water or salt lake water supplemented with sodium chloride to a desired molarity for growing D. salina. In an aspect, the molarity of the media may be increased using artificial sea salts or other salts known to those skilled in the art. In some embodiments the algae can be grown in a liquid environment which is 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3 molar or higher concentrations of sodium chloride. One of skill in the art will recognize that other salts (sodium salts, calcium salts, potassium salts, etc.) may also be present in the liquid environments.

Where a halophilic organism is used, it may be transformed with any vectors known in the art. For example, D. salina may be transformed with a vector which is capable of insertion into the nuclear genome and which contains nucleic acids which encode a flocculation moiety (e.g., an anti-cell-surface-protein antibody, a carbohydrate binding protein, etc.). Transformed halophilic organisms may then be grown in high-saline environments (e.g., salt lakes, salt ponds, high-saline media, etc.) to produce the products (e.g., isoprenoids, fatty acids, biomass degrading enzymes, etc.), or biomass, of interest. In some instances, a flocculation moiety may be non-functional under high salinity conditions. In such embodiments, flocculation may be induced by lowering the salinity (e.g., by diluting the liquid environment). Alternately, the flocculation moiety may be functional under high salinity conditions and flocculation may be controlled by increasing the salinity of the medium. Isolation of any products of interest produced by the organism may involve removing a transformed organism from a high-saline environment prior to extracting the product from the organism. In instances where the product is secreted into the surrounding environment, it may be necessary to desalinate the liquid environment prior to any further processing of the product.

Large scale culture can be conducted in a photobioreactor, semi-closed ponds, open ponds, or lakes. Multiple batches of small scale culture can be seeded into one large-scale culture vessel. The ratio of seeding volume to receiving volume can be determined at the time of seeding according to parameters such as optical density and growth rate of the small scale culture(s). In preparation of media for the large scale culture, autoclaving, adding nutrients to recycled media, evaluating the condition of recycled media, and measuring the pH, salt, and conductivity of the media can be performed. During the large scale culture, quality control is performed. Quality control criteria may include sampling and screening for contamination, strain divergence, growth kinetics, oxygen level, nitrogen level, salinity of the liquid, pH of the liquid media, sampling of growing cells for oil content measurement, dry weight/wet weight ratio, and optical density of the culture.

The present disclosure also provides for liquid systems having a controlled temperature. In one aspect, the temperature of the liquid system is maintained between 15° C. and 32° C. In another aspect, the temperature of the system is kept above 15° C. In yet another aspect, the temperature of the system is not allowed to exceed 32° C. In an aspect, the temperature of the system is kept below 25° C. In an aspect, the temperature may be from 0 to 35° C., from 5 to 35° C., from 10 to 35° C., 15 to 35° C., from 20 to 35° C., from 25 to 35° C., and from 30 to 35° C. In yet another aspect, the temperature may be maintained at greater than 5° C. In an aspect, the temperature may be maintained at greater than 10° C. In an aspect, the temperature may be maintained at greater than 15° C. In an aspect, the temperature may be maintained at greater than 20° C. or greater than 30° C. The present disclosure also provides for liquid systems having a temperature determined by the environment.

The microalgae may be grown in liquid systems of different volumes. In one aspect, the microalgae can be grown, for example, in small scale laboratory liquid systems. Small scale laboratory systems refer to cultures in volumes of less than about 6 liters. In an aspect, the small scale laboratory culture may be 1 liter, 2 liters, 3 liters, 4 liters, or 5 liters. In another aspect of the invention, the small scale laboratory culture may be less than one liter. In an aspect, the small scale laboratory culture may be 100 milliliters or less. In another aspect the culture may be 10 milliliter or less. In another aspect the liquid culture may be 5 milliliters or less. In yet another aspect, the liquid culture may be 1 milliliter or less.

In another aspect of the present disclosure, the liquid systems may be large scale cultures, where large scale cultures refers to growth of cultures in volumes of greater than about 6 liters, or greater than about 10 liters, or greater than about 20 liters. Large scale growth can also be growth of cultures in volumes of 50 liters or more, 100 liters or more, or 200 liters or more. Large scale growth can be growth of cultures in, for example, ponds, containers, vessels, or other areas, where the pond, container, vessel, or area that contains the culture is for example, at least 5 square meters, at least 10 square meters, at least 200 square meters, at least 500 square meters, at least 1,500 square meters, at least 2,500 square meters, in area, or greater.

The present disclosure further provides for very large scale liquid systems. In one aspect, the volume of liquid culture may be at least 20,000 liters. In another aspect, the volume of liquid can be up to 40,000 liters. In another aspect, the volume of liquid can be up to 80,000 liters. In another aspect, the volume of liquid can be up to 100,000 liters. In another aspect, the volume of liquid can be up to 150,000 liters. In another aspect, the volume of liquid can be up to 200,000 liters. In another aspect, the volume of liquid can be up to 250,000 liters. In another aspect, the volume of liquid can be up to 500,000 liters. In another aspect, the volume of liquid can be up to 600,000 liters. In another aspect, the volume of liquid can be up to 1,000,000 liters.

In yet another aspect, the very large scale liquid system may be from 10,000 to 20,000 liters. In an aspect, the very large scale liquid system may be from 10,000 to 40,000 liters or from 10,000 to 80,000 liters. In another aspect, the very large scale liquid system may be from 10,000 to 100,000 liters or from 10,000 to 150,000 liters. In yet another aspect, the liquid system may be from 10,000 to 200,000 liters or from 10,000 to 250,000 liters. The present disclosure also includes liquid systems from 10,000 to 500,000 liters or from 10,000 to 600,000 liters. The present disclosure further provides for liquid systems from 10,000 to 1,000,000 liters.

In further aspect, the liquid system may be from 20,000 to 40,000 liters or from 20,000 to 80,000 liters. In another aspect, the liquid system may be from 20,000 to 100,000 liters. In yet another aspect, the liquid system may be from 20,000 to 150,000 liters or from 20,000 to 200,000 liters. In another aspect, may be from 20,000 to 250,000 liters. In another aspect, the liquid system may be from 20,000 to 500,000 liters. In another aspect, the liquid system may be from 20,000 to 600,000 liters. In another aspect, the liquid system may be from 20,000 to 1,000,000 liters.

In another aspect, the liquid system may be from 40,000 to 80,000 liters. In another aspect, the liquid system may be from 40,000 to 100,000 liters. In another aspect, the liquid system may be from 40,000 to 150,000 liters. In another aspect, the liquid system may be from 40,000 to 200,000 liters. In another aspect, the liquid system may be from 40,000 to 250,000 liters. In another aspect, the liquid system may be from 40,000 to 500,000 liters. In another aspect, the liquid system may be from 40,000 to 600,000 liters. In another aspect, the liquid system may be from 40,000 to 1,000,000 liters.

In another aspect, the liquid system may be from 80,000 to 100,000 liters. In another aspect, the liquid system may be from 80,000 to 150,000 liters. In another aspect, the liquid system may be from 80,000 to 200,000 liters. In another aspect, the liquid system may be from 80,000 to 250,000 liters. In another aspect, the liquid system may be from 80,000 to 500,000 liters. In another aspect, the liquid system may be from 80,000 to 600,000 liters. In another aspect, the liquid system may be from 80,000 to 1,000,000 liters.

In another aspect, the liquid system may be from 100,000 to 150,000 liters. In another aspect, the liquid system may be from 100,000 to 200,000 liters. In another aspect, the liquid system may be from 100,000 to 250,000 liters. In another aspect, the liquid system may be from 100,000 to 500,000 liters. In another aspect, the liquid system may be from 100,000 to 600,000 liters. In another aspect, the liquid system may be from 100,000 to 1,000,000 liters.

In another aspect, the liquid system may be from 200,000 to 250,000 liters. In another aspect, the liquid system may be from 200,000 to 500,000 liters. In another aspect, the liquid system may be from 200,000 to 600,000 liters. In another aspect, the liquid system may be from 200,000 to 1,000,000 liters. In another aspect, the liquid system may be from 250,000 to 500,000 liters. In another aspect, the liquid system may be from 250,000 to 600,000 liters. In another aspect, the liquid system may be from 250,000 to 1,000,000 liters. In another aspect, the liquid system may be from 500,000 to 600,000 liters, or 500,000 to 1,000,000 liters.

In an aspect of the present disclosure, the liquid system may be a pond, either natural or artificial. In one aspect, the artificial pond may be a raceway pond. In a raceway pond, the organism, water, and nutrients circulate around a “racetrack.” Paddlewheels provide constant motion to the liquid in the racetrack, allowing for the organism to be circulated back to the surface of the liquid at a chosen frequency. Paddlewheels also provide a source of agitation and oxygenate the system. CO₂ may be added to a liquid system as a feedstock for photosynthesis through a CO₂ injection system. These raceway ponds can be enclosed, for example, in a building or a greenhouse, or can be located outdoors. In an aspect, an outdoor raceway liquid system may be enclosed with a cover, or exposed.

Raceway ponds are usually kept shallow because the organism needs to be exposed to sunlight, and sunlight can only penetrate the pond water to a limited depth. The depth of a raceway pond can be, for example, about 4 to about 12 inches. In addition, the volume of liquid that can be contained in a raceway pond can be, for example, about 200 liters to about 600,000 liters.

The raceway ponds can be operated in a continuous manner, with, for example, CO₂ and nutrients being constantly fed to the ponds, while water containing the organism is removed at the other end.

In an aspect, the ponds may have a surface area of at least 0.25 of an acre. In another aspect, the pond may be at least 0.5 acre or at least 1.0 acre. In yet another aspect, the pond may be at least 1.5 acres or at least 2.0 acres. The liquid system may be a pond of at least 2.5 acres or at least 5.0 acres. In an alternative aspect, the pond may be at least 7.5 acres or at least 10 acres. In still other embodiments, the pond may have a surface area of at least 12 acres, at least 15 acres, at least 18 acres, at least 20 acres, at least 25 acres, at least 30 acres, at least 35 acres, at least 40 acres, at least 45 acres or 50 acres.

In yet another aspect, the surface area of a pond may be from 0.25 to 0.5 acres or 0.25 to 1.0 acres. In an aspect, the liquid system may be a pond of 0.25 to 1.5 acres or 0.25 to 2.0 acres. In another aspect the pond may be from 0.25 to 2.5 acres, 0.25 to 5.0 acres or 0.25 to 7.5 acres. In yet another aspect, the liquid system may be a pond of 0.5 to 1.0 acres, 0.5 to 1.5 acres, 0.5 to 2.0 acres, 0.5 to 2.5 acres, 0.5 to 5.0 acres or 0.5 to 7.5 acres. In an aspect, the liquid system may cover an area of 1.0 to 1.5 acres or 1.0 to 2.0 acres. In an aspect, the liquid system may be a pond of 1.0 to 2.5 acres or 1.0 to 5.0 acres. In yet another aspect, the liquid system may be a pond of 1.0 to 7.5 acres or 2.0 to 2.5 acres. In another aspect the pond may be from 2.0 to 5.0 acres or 2.0 to 7.5 acres. In yet another aspect, the pond may range from 2.5 to 5.0 acres, 2.5 to 7.5 acres, 2.5 to 10 acres, 5 to 12 acres, 5 to 15 acres, 5 to 18 acres, 5 to 20 acres, 10 to 25 acres, 10 to 30 acres, 10 to 35 acres, 10 to 40 acres, 10 to 45 acres, or 10 to 50 acres in area.

Alternatively, organisms, such as microalgae, can be grown in closed structures such as photobioreactors, where the environment is under stricter control than in open systems or semi-closed systems. A photobioreactor is a bioreactor which incorporates some type of light source to provide photonic energy input into the reactor. The term photobioreactor can refer to a system closed to the environment and having no direct exchange of gases and contaminants with the environment. A photobioreactor can be described as an enclosed, illuminated culture vessel designed for controlled biomass production of phototrophic liquid cell suspension cultures. Examples of photobioreactors include, for example, glass containers, plastic tubes, tanks, plastic sleeves, and bags. Examples of light sources that can be used to provide the energy required to sustain photosynthesis include, for example, fluorescent bulbs, LEDs, and natural sunlight. Because these systems are closed everything that the organism needs to grow (for example, carbon dioxide, nutrients, water, and light) must be introduced into the bioreactor.

Photobioreactors, despite the costs to set up and maintain them, have several advantages over open systems, they can, for example, prevent or minimize contamination, permit axenic organism cultivation of monocultures (a culture consisting of only one species of organism), offer better control over the culture conditions (for example, pH, light, carbon dioxide, and temperature), prevent water evaporation, lower carbon dioxide losses due to out gassing, and permit higher cell concentrations.

On the other hand, certain requirements of photobioreactors, such as cooling, mixing, control of oxygen accumulation and biofouling, make these systems more expensive to build and operate than open systems or semi-closed systems.

Photobioreactors can be set up to be continually harvested (as is with the majority of the larger volume cultivation systems), or harvested one batch at a time (for example, as with polyethlyene bag cultivation). A batch photobioreactor is set up with, for example, nutrients, an organism (for example, microalgae), and water, and the organism is allowed to grow until the batch is harvested. A continuous photobioreactor can be harvested, for example, either continually, daily, or at fixed time intervals.

High density photobioreactors may be used and include those that are described in, for example, Lee, et al., Biotech. Bioengineering 44:1161-1167, 1994. Other types of bioreactors, such as those for sewage and waste water treatments, are described in, Sawayama, et al., Appl. Micro. Biotech., 41:729-731, 1994. Additional examples of photobioreactors are described in, U.S. Appl. Publ. No. 2005/0260553, U.S. Pat. No. 5,958,761, and U.S. Pat. No. 6,083,740. Also, organisms, such as microalgae may be mass-cultured for the removal of heavy metals (for example, as described in Wilkinson, Biotech. Letters, 11:861-864, 1989), hydrogen (for example, as described in U.S. Patent Application Publication No. 2003/0162273), and pharmaceutical compounds from a water, soil, or other source or sample. Organisms can also be cultured in conventional fermentation bioreactors, which include, but are not limited to, batch, fed-batch, cell recycle, and continuous fermenters. Additional methods of culturing organisms and variations of the methods described herein are known to one of skill in the art.

The present disclosure further provides for harvesting of the microalgae grown in the liquid system. Harvesting my accomplished by methods known to one of skill in the art including collection of the microalgae in whole or in part. In an aspect of the disclosure, harvesting may be accomplished by removing portions of the growing culture and separating the microalgae from the liquid. In another aspect, harvesting may be accomplished by continuous flow methods, for example, using a continuous flow centrifuge.

Separation of the microalgae from the liquid may be accomplished by methods known to one of ordinary skill in the art. In one aspect, the microalgae may be allowed to settle by gravity and the overlying liquid removed. In another aspect, the microalgae may be harvested by centrifugation of the microalgae containing culture. In an aspect, centrifugation of the liquid culture may be performed in batch mode, using a fixed volume centrifuge. In a different aspect, batch harvesting of the microalgae may be accomplished using a continuous flow centrifuge. In another aspect, the microalgae may be harvested continuously from the growing culture by continuous flow centrifugation.

In one aspect of the present disclosure, harvesting of the microalgae grown in the liquid system may be facilitated by flocculation. Methods for inducing flocculation include those that can be found in U.S. Patent Publication No. US 2011/0159595, application Ser. No. 13/001,027, hereby incorporated in its entirety by reference. The flocculate may be separated from the culture liquid by gravity, centrifugation or other physical method known to those of skill in the art. In a particular embodiment the flocculate may be separated form the culture liquid by dissolved air flotation (DAF).

The present disclosure provides for harvesting of all or part of the liquid culture system. In an aspect, harvesting includes separating at least 90% of the microalgae from the liquid culture to produce a microalgae depleted liquid. In another aspect, at least 95% of the microalgae are removed from the liquid culture. In another aspect, at least 97% of the microalgae are removed from the liquid culture. In another aspect, at least 99% of the microalgae are removed from the liquid culture. In other aspects, 50% or more of the microalgae are removed. In another aspect, 75% or more of the microalgae are removed from the liquid culture. In still another aspect, 80% of more of the microalgae are removed from the liquid culture. In yet another aspect, the liquid culture can have less than 30% of the microalgae remaining after harvesting. In a further aspect, less than 25% of the microalgae remained after harvesting. In a further aspect, less than 5% of the microalgae remained after harvesting. In a further aspect, less than 2.5% of the microalgae remained after harvesting. In an aspect, less than 1% of the microalgae remain after harvesting.

In a further aspect of the invention, less than 10⁵ microalgae cells per milliliter remain in the liquid after harvesting (10⁵ cells/ml). In another aspect, after harvesting, less than 10⁴ cells/ml remain in the liquid. In yet another aspect, less than 10³ cells/ml remain in the liquid after harvesting. In a further aspect, 10² cells/ml remain in the liquid after harvest.

In an aspect, harvesting of microalgae from the growing culture may be performed on a part of the total liquid culture. In one aspect, the part of the liquid culture is removed and the microalgae are harvested. In an aspect, at least 2 percent of a total volume of a liquid culture is removed and the microalgae harvested. In another aspect, at least 2.5% of the total volume of the liquid culture containing the growing microalgae is removed and the microalgae harvested. In an aspect, at least 5% or at least 7.5% of the total volume of the liquid culture containing the growing microalgae is removed for harvesting. In yet another aspect, at least 10% or at least 12.5% of the total volume of the liquid culture containing the growing microalgae is removed for harvesting. In a further aspect, at least 15% or at least 20% of the total volume of the liquid culture containing the growing microalgae is removed for harvesting.

In a further aspect, from 2 to 5% or from 2 to 7.5% of the total volume of the liquid culture containing the growing microalgae is removed for harvesting. In another aspect from 2 to 20% or from 2 to 12.5% of the total volume of the liquid culture containing the growing microalgae is removed for harvesting. In an aspect, the amount of liquid removed for harvesting may range from 2 to 15% or from 2 to 20% of the total volume of the liquid culture. In a further aspect, from 2.5 to 5% or from 2.5 to 7.5% of the total liquid culture volume may be removed for harvesting. In an aspect, the amount of liquid removed for harvesting may be from 2.5 to 10% or from 2.5 to 12.5% of the total growing culture volume. In an aspect, the amount removed may range from 2.5 to 15% or from 2.5 to 20%. In a further aspect, from 5 to 7.5% or from 5 to 10% of the culture volume may be removed for harvesting. In an aspect, from 5 to 12.5%, from 5 to 15%, or even from 5 to 20% of the total volume of liquid culture may be harvested. In another aspect, the amount of harvested culture may be from 7.5 to 10% or from 7.5 to 12.5% of the total culture volume. In an aspect, the amount of liquid removed for harvesting my range from 7.5 to 15% or from 7.5 to 20% of the culture volume. In yet another aspect, 10 to 12.5% or 10 to 15% of the culture volume may be removed from harvesting. In an aspect, 10 to 20% of the total volume of a liquid culture may be removed for harvesting of the growing microalgae.

It is further provided as part of the present disclosure that harvesting may be conducted continuously from the growing culture of microalgae. In an aspect, removal of the microalgae maintains the culture in a logarithmic phase of microalgae growth. One of skill in the art understands that the when growing in a logarithmic phase, the number of microalgae double within a time period. The time period for microalgae doubling depends on the environment of the growing microalgae. The determination of growth rates and phases of microalgae growth are known in the art. For example, in Sode et al., “On-line monitoring of marine cyanobacterial cultivation based on phycocyanin fluorescence,” J. Biotechnology 21:209-217 (1991), Torzillo et al., “On-Line Monitoring Of Chlorophyll Fluorescence To Assess The Extent Of Photoinhibition Of Photosynthesis Induced By High Oxygen Concentration And Low Temperature And Its Effect On The Productivity Of Outdoor Cultures Of Spirulina Platensis (Cyanobacteria),” J. Phycology 34:504-510 (1998), Jung and Lee, “In Situ Monitoring of Cell Concentration in a Photobioreactor Using Image Analysis: Comparison of Uniform Light Distribution Model and Artificial Neural Networks” Biotechnology Progress 22:1443-1450 (2006), and Vonshak, A. Spirulina Platensis Arthrospira: Physiology, Cell-Biology And Biotechnology. 1997. CRC Press, all of which are incorporated by reference in their entireties. In an aspect, harvesting may be performed when microalgae are in logarithmic phase growth as provided further herein.

In an aspect, a portion of the liquid culture may be removed for harvesting and the portion replaced so that the total volume of the liquid culture remains within a narrow range. In one aspect, the amount of liquid removed during continuous harvesting is up to 1000 gallons per hour. In another aspect, the amount removed during continuous harvesting may be 1% of the total volume per hour. In an aspect, up to 5% of the volume per day may be removed during a continuous harvesting. In an aspect, up to 15% of the volume per day may be removed during a continuous harvesting. In an aspect, up to 33% of the volume per day may be removed during a continuous harvesting.

The present disclosure further provides for recycling of the liquid after harvesting. In one aspect, the liquid may be returned to the liquid culture system and recycled. Recycling of the liquid provides for the conservation of the water and may improve efficiency. Recycling of media (e.g., laboratory media, pond water, lake water, bioreactor contents, etc.) is economically advantageous, especially in large scale operations. For example, in a controlled circulating pond system, the liquid environment can be recycled by allowing continuous flow of the liquid while nutrients are continuously added. In another aspect, in a closed photobioreactor system, media recycling may comprise scooping out flocculated NVPO mass. In an aspect, the liquid for recycling, the pH of the liquid may be measured and adjusted. In another aspect, the levels of nutrients may be measured. In a further aspect, the measured nutrients may be adjusted to preferred or optimal levels. In yet another aspect, the liquid may be sterilized by autoclaving or by treatment with a chemical or by treatment by UV irradiation. In one aspect, the recycled liquid may be returned directly to the liquid culture system without modification or addition. In an aspect, the recycled liquid may be treated to remove contaminants that are detrimental to the growth of microalgae. In an aspect, a contaminant may be or eukaryotic or prokaryotic pest. A contaminant may be a direct pest, for example a chytrids, or an indirect pest, for example, a Halomonas species of bacteria.

In an aspect, the recycled liquid may contain microalgae. In an aspect, removal of all the growing microalgae during the harvesting step is not required prior to returning the liquid to the liquid culture system. In an aspect, incomplete removal decreases the amount of time necessary to recycle the liquid.

In another aspect, a polymer is introduced to the culture during the harvest process to induce flocculation. In an aspect, less than complete removal of the flocculated microalgae provides for less residual polymer when the liquid is returned to the liquid culture system. Residual polymer in a return feed to a liquid system may reduce productivity by inducing low grade flocculation in the pond culture.

The present disclosure further provides for other uses of the microalgae depleted liquid culture other than returning a recycled liquid to the growing microalgae culture. In one aspect, a recycled liquid may be used for crop irrigation. In another aspect, a recycled liquid can be used in other industrial processes. In yet another aspect, a recycled liquid may be discharged into an existing body of water. In an aspect a recycled liquid may be discharged to an evaporation pond. In an aspect, a recycled liquid may be used in other microbial driven processes such as fermentation and other methods to reclaim nutrients.

The present disclosure provides for liquid culture systems that are either indoors or outdoors. The advantage of an indoor system may that the environment may be more easily controlled. In an aspect, the temperature of an indoor environment may be regulated. In another aspect, the amount and quality of the light may be controlled. In one aspect, an indoor system may be a greenhouse. In an aspect, a greenhouse may receive natural light. In another aspect a greenhouse may be artificially lighted. In yet another aspect, natural light may be supplemented by artificial light.

In an aspect, artificial light may be fluorescent light. One source of energy is fluorescent light that can be placed, for example, at a distance of about 1 inch to about two feet from the organism. Examples of types of fluorescent lights includes, for example, cool white and daylight. If the lights are turned on and off at regular intervals (for example, 12:12 or 14:10 hours of light:dark) the cells of some organisms will become synchronized.

Growth of micro-organisms in general proceeds along known phases and this is true for the microalgae of the present disclosure. When a liquid culture is inoculated with a microalgae, there is often a ‘lag phase’ during which changes in the density of the organism are not readily detectable. Following the lag phase, the organism enters and early growth phase characterized by increasing density of the microorganism.

An early growth phase is followed by a logarithmic growth phase during which many of the microorganisms are dividing. The logarithmic growth phase is characterized by log-linear growth of the organism when the density or cell number is plotted on a logarithmic scale versus time. The ‘doubling’ time is used to characterize this phase of growth. Both extrinsic environmental factors and intrinsic factors control the doubling time of an organisms. Those of skill in the art recognize that the rate of doubling can be limited by the necessity of initiating and completing successive rounds of DNA synthesis and genome replication. This limit on doubling time can be observed when all extrinsic environmental factors are non-limiting. Extrinsic factors play important roles in the growth of microalgae including the presence of nutrients, the temperature, the pH, and the availability of light for photosynthesis. Methods of growing and optimizing the growth of microalgae are known in the art, for example in Vonshak, A. Spirulina Platensis Arthrospira: Physiology, Cell-Biology And Biotechnology. 1997. CRC Press and M. Tredici, “Photobiology of microalgae mass cultures: understanding the tools for the next green revolution,” Biofuels 1:143 (2010), both of which are hereby incorporated by reference in their entireties.

As the density increases, the rate of doubling decreases in a phase called “late log-phase.” Growth decreases due to limiting nutrients (for example, lack of CO₂, lack of a carbon source etc.) or is due to factors secreted by the growing organisms (e.g., quorum sensing).

At the end of the log-phase of growth, the number of microorganisms stops increasing and the culture enters a stationary phase. In some aspects, the microorganisms may initiate developmental pathways leading, for example, a quiescent state. In another aspect, the microorganisms may have changes in gene expression including both increases and decreases in the expression. Removal of microorganisms in the stationary phase and inoculation of a fresh culture often results in a lag phase prior to entry into a logarithmic growth phase.

The doubling time during growth in the logarithmic phase can depend on a number environmental conditions. Among the factors it is recognized that the nutrients and media conditions significantly affect growth. In the present disclosure, microalgae can be autotrophic and are therefore less susceptible to the presence of carbon based food sources. One of ordinary skill in the art would understand that the availability of nitrogen affects microalgae growth. Decreased nitrogen leads to longer doubling times, or even entry into stationary phases. Increased nitrogen availability may result in decreased doubling time. In an aspect, a growing liquid culture can be monitored for changes in the environmental conditions to maintain or optimize logarithmic phase growth. Production of microalgae is optimized when growth is logarithmic.

In an aspect, the growth of the culture proceeds through different growth phases. In one aspect, a liquid culture is inoculated and proceeds from a lag phase to the logarithmic phase to the stationary phase. In another aspect, logarithmically growing microalgae are provided such that there is no lag phase of growth. In another aspect, logarithmic phase is maintained by harvesting microalgae. In a further aspect, logarithmic phase is maintained by supplementing the liquid culture system that is limited for one or more nutrients.

In an aspect, a logarithmic growth phase is maintained by harvesting microalgae and supplementing the liquid culture system. In one aspect, a liquid after harvest can be monitored and nutrients added prior to returning the liquid culture system. In another aspect, a liquid culture system can be supplied with fresh media, for example water, and logarithmic phase maintained. In an aspect, a fresh media may contain nutrients necessary to maintain the logarithmic phase of microalgae growth. In a further aspect, microalgae depleted liquid can be further purified to remove contaminants to maintain logarithmic growth.

In an aspect, the liquid culture is treated with fungicide during the logarithmic phase. In another aspect of the invention, the liquid culture is treated during the lag phase.

In an aspect, the liquid culture is treated during the stationary phase. In an aspect, the microalgae are harvested from the liquid culture during logarithmic phase. In an aspect, the microalgae are harvested from the liquid culture during late logarithmic phase. In another aspect, the microalgae are harvested from the liquid culture during stationary phase. In an aspect, algae growth is maintained at an optimal density for logarithmic growth. In an aspect, the optimal density may be determined experimentally for a strain of microalgae.

Testing for the presence of a pest need not be conducted at any particular phase of growth. Thus, the present disclosure provides for testing for the presence of a pest of a liquid system at any phase of growth of a microalgae culture. In an aspect, testing for the presence of a pest may be performed before inoculation of the liquid system with a microalga. In another aspect, testing may be performed during the lag phase of microalgae growth. In yet another aspect, testing may be performed during logarithmic growth or at late logarithmic growth. In an aspect, testing may be performed at a stationary phase of a microalgae growth cycle. In yet another aspect, testing may be performed throughout each stage of a microalgae growth cycle.

The present disclosure provides for treating a liquid system contaminated with a pest at any phase of growth of a microalgae culture and at multiple stages of growth. In an aspect, treatment may be performed before inoculation of the liquid system with microalgae. In another aspect, treatment may be performed during the lag phase of microalgae growth. In yet another aspect, treatment may be performed during logarithmic growth or at late logarithmic growth. In an aspect, treatment may be performed at a stationary phase of a microalgae growth cycle. In yet another aspect, treatment may be performed throughout each stage of a microalgae growth cycle.

In one aspect, a liquid culture is grown for 15 or more days. In another aspect, a liquid culture is grown for 30 or more days. In an aspect, a liquid culture is grown for 45 or more days. In another aspect, a liquid culture is grown 60 or more, or 90 or more days. In yet another aspect, growth time may be 120 or more, or 180 or more days. In an aspect, a liquid culture may be maintained 250 or more, or 500 or more days. In yet another aspect, growth of a liquid culture may be continued for 1000 or more, 1500 or more, or 2000 or more days after inoculation of the liquid culture. The culture may be maintained, with fungicide treatments of the present disclosure for an indefinite amount of time.

The present disclosure provides for treatments of a liquid system. Treatments may include physical methods to control the growth of, or kill a pest present in a liquid system. Physical methods may include, as non limiting examples, filtration, heating, cooling and irradiation.

The present disclosure provides for treatments of a liquid system including the addition of compositions that control the growth of, or kill a pest. In an aspect, the treatment may be provided upon detecting the presence of a pest. In an aspect, the treatment may be provide upon the detection of a fungus in a liquid system. In a further aspect, the treatment may be prophylactic and the treatment may be provided during any stage of growth of the microalgae.

Treatments of the present disclosure include adding one or more fungicides to a liquid culture system. In an aspect, a fungicide may be a chemical compound. In an aspect, the fungicide may further contain non-active ingredients that aid in dissolving or dispensing the active ingredient. Fungicides may be known in the art or may be developed to kill or inhibit a pest. Non-limiting examples of fungicides of the present disclosure are presented in Table 1.

Treatments of the present disclosure include providing one or more fungicides presented in Table 1. In an aspect, a first effective concentration of fungicide may be provided to a liquid system upon detection of a first pest. In another aspect, an effective concentration of second fungicide may be provided to a liquid system where the growth of a first pest is not inhibited relative to the growth of a first pest without a first fungicide. In another aspect, an effective concentration of second fungicide may be provided to a liquid system after the effective concentration of the first fungicide and upon detection of a pest. In an aspect, a fungicide is selected to have a different mechanism of action than a first fungicide. In a further aspect, a third fungicide may be provided as a treatment of a liquid system after the effective treatment of a first and second fungicide. In yet another aspect, a first, second and third fungicide may be rotated to ensure effective control of a pest in a liquid culture system and to avoid the development of fungicide resistance in a liquid culture system.

In an aspect of the present disclosure, a combination of two fungicides may be provided upon detection of a first pest. In yet another aspect, a third fungicide may be provided where the first and second fungicide combination does not control a pest of the liquid system.

TABLE 1
Fungicide Sources and Mechanisms of Action

| | Sigma- | |
|---|---|---|
| | Aldrich ® | |
| Description | catalog # | MOA |
| acibenzolar | 32820 | Host plant defense induction; |
| | | Salicylic acid pathway |
| azoxystrobin | 31697 | Respiration; QOI-fungicide |
| | | (Quinone outside Inhibitors) |
| benodanil | 45338 | respiration; SDHI (Succinate |
| | | dehydrogenase inhibitors) |
| binapacryl | 31484 | Sterol biosynthesis in membranes, |
| | | uncouplers of oxidative |
| | | phosphorylation |
| boscalid | 33875 | Respiration; SDHI |
| bronopol | 32053 | |
| captan | 32054 | Multi site contact activity |
| carbendazim | 45368 | Mitosis and Cell Division; MBC - |
| | | fungicides (Methyl-Benzimidazole |
| | | Carbamates) |
| carboxine | 45371 | respiration; SDHI |
| chlorothalonil | 36791 | Multi site Contact Activity |
| cyazofamid | 33874 | respiration; QOI-fungicides |
| cymoxanil | 34326 | Unknown Mode of action |
| cyprodinil | 34389 | Amino Acids and Protein Synthesis; |
| | | Methionine biosynthesis |
| dibromocyano- | 540978 | |
| acetamide | | |
| dimoxystrobin | 33499 | Respiration; QOI-fungicides |
| dinocap | 45452 | Respiration; Uncouplers of oxidative |
| | | phosphorylation |
| diquat | | |
| dibromide | | |
| dithianon | 45462 | Multi site contact activity |
| dodemorph | 45465 | Sterol biosynthesis in membranes; |
| | | reductase and isomerase in sterol |
| | | biosynthesis |
| dodine | PS250 | Unknown Mode of action; Cell |
| | | Membrane disruption |
| endothal | 35525 | |
| monohydrate | | |
| fenarimol | 45484 | Sterol biosynthesis in membranes; |
| | | DMI fungicides (Demethylation |
| | | inhibitors) |
| fenhexamid | 31713 | Sterol biosynthesis; 3-keto reductase, |
| | | C4- de-methylation |
| fenpropidin | 46017 | sterol biosynthesis in membranes; |
| | | D14-reductase and. isomerase in |
| | | sterol biosynthesis (erg24, erg2) |
| fluazinam | 34095 | Respiration; uncouplers of oxidative |
| | | phosphorylation |
| fluoxastrobin | 33797 | Respiration; QOI-fungicides |
| fosetyl- | PS2026 | Unknown MOA |
| aluminum | | |
| (100 mg) | | |
| kresoxim- | 37899 | Respiration; QOI-fungicides |
| methyl | | |
| mancozeb | 45553 | Multi site Contact Activity |
| metalaxyl | 32012 | Nucleic Acid Synthesis; PA - |
| | | fungicides (PhenylAmides) |
| methyl | 73569 | |
| isothiazolin | | |
| nystatin | N4014 | Sterol biosynthesis in membranes |
| oryzalin | 36182 | Microtubule assembly inhibition |
| pencycuron | 31118 | Mitosis and cell division; cell |
| | | division |
| propamocarb | 45638 | lipids and membrane synthesis; cell |
| | | membrane permeability, fatty acids, |
| | | carbamate |
| propiconazole | 45642 | Sterol biosynthesis in membranes; |
| | | DMI fungicides |
| prothioconazole | 34232 | Sterol biosynthesis in membranes; |
| | | DMI fungicides |
| pyraclostrobin | 33696 | Respiration; QOI-fungicides |
| pyrifenox | 45737 | Sterol biosynthesis in membranes; |
| | | DMI fungicides |
| sonar | chem | |
| | service | |
| spiroxamine | 46443 | Sterol biosynthesis in membranes; |
| | | reductase and isomerase in sterol |
| | | biosynthesis |
| tebuconazole | 32013 | Sterol biosynthesis in membranes; |
| | | DMI fungicides |
| temefos | 31526 | |
| terbuthylazine | 45678 | |
| thiophanate- | 45688 | Mitosis and Cell Division; MBC - |
| methyl | | fungicides |
| Thiram ® | 45689 | Multi site Contact Activity |
| tolylfluanid | 32060 | Multi site contact activity |
| triadimenol A | 45694 | Sterol biosynthesis in membranes; |
| | | DMI fungicides |
| triclopyr | 32016 | |
| trifloxystrobin | 46447 | Respiration; QOI-fungicide |
| triflumizole | 32611 | Sterol biosynthesis in membranes; |
| | | DMI fungicides |
| trifluralin | 32061 | |
| triforin | 45701 | Sterol biosynthesis in membranes; |
| | | DMI fungicides |
| zoxamide | 32501 | Mitosis and cell division; β-tubulin |
| | | assembly in mitosis |

In an aspect, the treatments may be performed at a specified time of the day. In an aspect, the treatment may be conducted in the morning. In another aspect, the treatment may be conducted at mid-day. In yet another aspect, the treatment may be performed at or near sunset. In another aspect, treatment may be performed at night. In one aspect, treatment may be performed at two periods each day, for example in the morning and again in the evening. In another aspect, treatment may occur during the day and a second monitoring may occur at night.

The present disclosure provides for the treatment of a liquid system to minimize the formation of concentration gradients. In an aspect, an amount of treatment is calculated based on the volume of a liquid system and prepared in a volume of the media (e.g. the culture media of the liquid system) to prepare a concentrated treatment stock. A concentrated treatment stock may be slowly added to a liquid system. In an aspect, concentrated treatment stock is added behind a paddle wheel of a raceway pond system. In another aspect, the concentrated treatment stock is dispersed by spraying of a liquid system. In yet another embodiment, the concentrated treatment stock is added to a water return line of a circulation pump.

In an aspect, the treatment of a liquid system may be monitored by obtaining samples of a liquid system for analysis using High Performance Liquid Chromotagraphy (HPLC). In an aspect, a time series of samples is obtained and filtered to remove particulate matter (e.g., growing microalgae) and then stored at −20° C. until analyzed using HPLC. In an aspect, samples are collected every 12 hours. In another aspect, samples are collected every 24 hours. In yet another aspect, samples are collected at 48 hours.

The present disclosure further provides for providing a treatment to a liquid system when the liquid system attains a specified temperature. In an aspect, treatment of a liquid system may be provided when the temperature if the liquid is below 25° C. In another aspect, a treatment may be provided when the temperature of the liquid is above 25° C. In aspect, a treatment may be provided when the temperature of the liquid is below 37° C. In yet another aspect, a treatment may be provided when the temperature of the liquid system is between 25 and 37° C. In an aspect, the temperature of the liquid may be between 0 and 15° C. or between 15 and 25° C. In a further aspect, the temperature of the liquid may be between 15 and 37° C. In yet another aspect, a treatment may be provided when the temperature of a liquid system may be below 37° C. In an aspect, the temperature of a liquid system may be below 32° C. In an aspect, the temperature of a liquid system of the present invention may be below 25° C. In an aspect, the temperature of a liquid system may be below 25° C. The present disclosure further provides for determining an optimal temperature for providing a treatment of the present disclosure based on the chemical properties of the pesticide or fungicide.

The present disclosure provides for the treatment of a liquid system with a fungicide of the pyridinamine family. In an aspect, the pyridinamine may be fluazinam (phenyl-pyridinamine or 3-chloro-N-[3-chloro-2,6-dinitro-4-(trifluoromethyl)phenyl]-5-(trifluoromethyl)-2-pyridinamine (CAS No. 79622-59-6)). In an aspect, fluazinam may be provided as a first fungicide treatment of a liquid system. In another aspect, fluazinam may be provided as a second fungicide treatment. In an aspect, fluazinam may be provided as a third fungicide treatment. In yet another aspect, fluazinam may be provided as fourth treatment or a fifth treatment. In another aspect, fluazinam may be provided as sixth treatment or a seventh treatment. In other embodiments, fluazinam may be administered in combination with one or more fungicides either separately by being administered contemporaneously with the one or more fungicides, or as part of a mixture of fungicides.

The present disclosure provides for the treatment of a liquid system with a fungicide of the methoxy-carbamate family. In an aspect, the methoxy-carbamate may be pyraclostrobin (methyl N-[2-[[[1-(4-chlorophenyl)-1H-pyrazol-3-yl]oxy]methyl]phenyl]-N-methoxycarbamate (CAS No. 175013-18-0). In an aspect, pyraclostrobin may be provided as a first fungicide treatment of a liquid system. In another aspect, pyraclostrobin may be provided as a second fungicide treatment. In an aspect, pyraclostrobin may be provided as a third fungicide treatment. In yet another aspect, pyraclostrobin may be provided as fourth treatment or a fifth treatment. In another aspect, pyraclostrobin may be provided as sixth treatment or a seventh treatment. In other embodiments, pyraclostrobin may be administered in combination with one or more fungicides either separately by being administered contemporaneously with the one or more fungicides, or as part of a mixture of fungicides.

The present disclosure further provides for the treatment of a liquid system with a fungicide of the dithiocarbamate family. In an aspect, the dithiocarbamate may be Thiram® (tetramethylthioperoxydicarbonic diamide (CAS No. 137-26-8). In an aspect, Thiram® may be provided as a first fungicide treatment of a liquid system. In another aspect, Thiram® may be provided as a second fungicide treatment. In an aspect, Thiram® may be provided as a third fungicide treatment. In yet another aspect, Thiram® may be provided as fourth treatment or a fifth treatment. In another aspect, Thiram® may be provided as sixth treatment or a seventh treatment. In other embodiments, Thiram® may be administered in combination with one or more fungicides either separately by being administered contemporaneously with the one or more fungicides, or as part of a mixture of fungicides.

The present disclosure further provides for the treatment of a liquid system with a fungicide of the benzothiadiazole family. In an aspect, the benzothiadiazole may be acibenzolar (benzo(1,2,3)thiadiazole-7-carbothioic acid-5-methyl ester (CAS No. 135158-54-2)). In an aspect, acibenzolar may be provided as a first fungicide treatment of a liquid system. In another aspect, acibenzolar may be provided as a second fungicide treatment. In an aspect, acibenzolar may be provided as a third fungicide treatment. In yet another aspect, acibenzolar may be provided as fourth treatment or a fifth treatment. In another aspect, acibenzolar may be provided as sixth treatment or a seventh treatment. In other embodiments, acibenzolar may be administered in combination with one or more fungicides either separately by being administered contemporaneously with the one, or more fungicides or as part of a mixture of fungicides.

The present disclosure further provides for the treatment of a liquid system with a fungicide of the anilide family. In an aspect, the anilide may be benodanil (2-Iodo-N-phenylbenzamide (CAS No. 15310-01-7)). In an aspect, benodanil may be provided as a first fungicide treatment of a liquid system. In another aspect, benodanil may be provided as a second fungicide treatment. In an aspect, benodanil may be provided as a third fungicide treatment. In yet another aspect, benodanil may be provided as fourth treatment or a fifth treatment. In another aspect, benodanil may be provided as sixth treatment or a seventh treatment. In other embodiments, benodanil may be administered in combination with one or more fungicides either separately by being administered contemporaneously with the one or more fungicides, or as part of a mixture of fungicides.

The present disclosure further provides for the treatment of a liquid system with the fungicide bronopol (2-bromo-2-nitropropane-1,3-diol (CAS No. 52-51-7)). In an aspect, bronopol may be provided as a first fungicide treatment of a liquid system. In another aspect, bronopol may be provided as a second fungicide treatment. In an aspect, bronopol may be provided as a third fungicide treatment. In yet another aspect, bronopol may be provided as fourth treatment or a fifth treatment. In another aspect, bronopol may be provided as sixth treatment or a seventh treatment. In other embodiments, bronopol may be administered in combination with one or more fungicides either separately by being administered contemporaneously with the one or more fungicides, or as part of a mixture of fungicides.

The present disclosure further provides for the treatment of a liquid system with the fungicide carbendazim (N-1H-(Benzimidazol-d4)-2-yl-carbamic Acid Methyl Ester (CAS No. 291765-95-2)). In an aspect, carbendazim may be provided as a first fungicide treatment of a liquid system. In another aspect, carbendazim may be provided as a second fungicide treatment. In an aspect, carbendazim may be provided as a third fungicide treatment. In yet another aspect, carbendazim may be provided as fourth treatment or a fifth treatment. In another aspect, carbendazim may be provided as sixth treatment or a seventh treatment. In other embodiments, carbendazim may be administered in combination with one or more fungicides either separately by being administered contemporaneously with the one or more fungicides, or as part of a mixture of fungicides.

The present disclosure further provides for the treatment of a liquid system with the fungicide oxathiins (carboxine 6-methyl-N-phenyl-2,3-dihydro-1,4-oxathiine-5-carboxamide). In an aspect, oxathiins may be provided as a first fungicide treatment of a liquid system. In another aspect, oxathiins may be provided as a second fungicide treatment. In an aspect, oxathiins may be provided as a third fungicide treatment. In yet another aspect, oxathiins may be provided as fourth treatment or a fifth treatment. In another aspect, oxathiins may be provided as sixth treatment or a seventh treatment. In other embodiments, oxathiins may be administered in combination with one or more fungicides either separately by being administered contemporaneously with the one or more fungicides, or as part of a mixture of fungicides.

The present disclosure further provides for the treatment of a liquid system with a fungicide of the nitrile family. In an aspect, the nitrile may be chlorothalonil (2,4,5,6-tetrachlorobenzene-1,3-dicarbonitrile). In an aspect, chlorothalonil may be provided as a first fungicide treatment of a liquid system. In another aspect, chlorothalonil may be provided as a second fungicide treatment. In an aspect, chlorothalonil may be provided as a third fungicide treatment. In yet another aspect, chlorothalonil may be provided as fourth treatment or a fifth treatment. In another aspect, chlorothalonil may be provided as sixth treatment or a seventh treatment. In an aspect, the nitrile may be dibromocyanoacetamide(2,2-dibromo-2-cyanoacetamide). In an aspect, dibromocyanoacetamide may be provided as a first fungicide treatment of a liquid system. In another aspect, dibromocyanoacetamide may be provided as a second fungicide treatment. In an aspect, dibromocyanoacetamide may be provided as a third fungicide treatment. In yet another aspect, dibromocyanoacetamide may be provided as fourth treatment or a fifth treatment. In another aspect, dibromocyanoacetamide may be provided as sixth treatment or a seventh treatment. In other embodiments, dibromocyanoacetamide may be administered in combination with one or more fungicides either separately by being administered contemporaneously with the one or more fungicides, or as part of a mixture of fungicides.

The present disclosure further provides for the treatment of a liquid system with a fungicide of the pyrimidine family. In an aspect, the pyrimidine may be cyprodinil (4-Cyclopropyl-6-methyl-N-phenylpyrimidin-2-amine). In an aspect, cyprodinil may be provided as a first fungicide treatment of a liquid system. In another aspect, cyprodinil may be provided as a second fungicide treatment. In an aspect, cyprodinil may be provided as a third fungicide treatment. In yet another aspect, cyprodinil may be provided as fourth treatment or a fifth treatment. In another aspect, cyprodinil may be provided as sixth treatment or a seventh treatment. In other embodiments, cyprodinil may be administered in combination with one or more fungicides either separately by being administered contemporaneously with the one or more fungicides, or as part of a mixture of fungicides.

The present disclosure further provides for the treatment of a liquid system with a fungicide of the pyridine family. In an aspect, the pyridine may be diquat dibromide (9,10-Dihydro-8a,10a-diazoniaphenanthrene(1,1′-ethylene-2,2′-bipyridylium)dibromide). In an aspect, diquat dibromide may be provided as a first fungicide treatment of a liquid system. In another aspect, diquat dibromide may be provided as a second fungicide treatment. In an aspect, diquat dibromide may be provided as a third fungicide treatment. In yet another aspect, diquat dibromide may be provided as fourth treatment or a fifth treatment. In another aspect, diquat dibromide may be provided as sixth treatment or a seventh treatment. In other embodiments, diquat dibromide may be administered in combination with one or more fungicides either separately by being administered contemporaneously with the one or more fungicides, or as part of a mixture of fungicides.

The present disclosure further provides for the treatment of a liquid system with a fungicide of the anthraquinones family. In an aspect, the anthraquinones may be dithianon (5,10-dioxobenzo[g][1,4]benzodithiine-2,3-dicarbonitrile (CAS No. 347-22-6)). In an aspect, dithianon may be provided as a first fungicide treatment of a liquid system. In another aspect, dithianon may be provided as a second fungicide treatment. In an aspect, dithianon may be provided as a third fungicide treatment. In yet another aspect, dithianon may be provided as fourth treatment or a fifth treatment. In another aspect, dithianon may be provided as sixth treatment or a seventh treatment. In other embodiments, dithianon may be administered in combination with one or more fungicides either separately by being administered contemporaneously with the one or more fungicides or as part of a mixture of fungicides.

The present disclosure further provides for the treatment of a liquid system with a fungicide of the aliphatic nitrogen fungicides family. In an aspect, the aliphatic nitrogen fungicide may be dodine (dodecylguanidinium acetate (CAS No. 2439-10-3)). In an aspect, dodine may be provided as a first fungicide treatment of a liquid system. In another aspect, dodine may be provided as a second fungicide treatment. In an aspect, dodine may be provided as a third fungicide treatment. In yet another aspect, dodine may be provided as fourth treatment or a fifth treatment. In another aspect, dodine may be provided as sixth treatment or a seventh treatment. In other embodiments, dodine may be administered in combination with one or more fungicides either separately by being administered contemporaneously with the one or more fungicides, or as part of a mixture of fungicides. In yet another aspect, the chloride salt of dodecylguanidine may be used (e.g., dodecylguanidinium hydrochloride, CAS No. 13590-91-1)).

The present disclosure further provides for the treatment of a liquid system with the fungicide fenarimol (2-chlorophenyl)-(4-chlorophenyl)-pyrimidin-5-ylmethanol (CAS No. 60168-88-9). In an aspect, fenarimol may be provided as a first fungicide treatment of a liquid system. In another aspect, fenarimol may be provided as a second fungicide treatment.

In an aspect, fenarimol may be provided as a third fungicide treatment. In yet another aspect, fenarimol may be provided as fourth treatment or a fifth treatment. In another aspect, fenarimol may be provided as sixth treatment or a seventh treatment. In other embodiments, fenarimol may be administered in combination with one or more fungicides either separately by being administered contemporaneously with the one or more fungicides, or as part of a mixture of fungicides.

The present disclosure further provides for the treatment of a liquid system with the fungicide fenpropidin (1-[3-(4-tert-butylphenyl)-2-methylpropyl]piperidine (CAS No. 67306-00-7)). In an aspect, fenpropidin may be provided as a first fungicide treatment of a liquid system. In another aspect, fenpropidin may be provided as a second fungicide treatment. In an aspect, fenpropidin may be provided as a third fungicide treatment. In yet another aspect, fenpropidin may be provided as fourth treatment or a fifth treatment. In another aspect, fenpropidin may be provided as sixth treatment or a seventh treatment. In other embodiments, fenpropidin may be administered in combination with one or more fungicides either separately by being administered contemporaneously with the one or more fungicides, or as part of a mixture of fungicides.

The present disclosure further provides for the treatment of a liquid system with the fungicide propiconazole (1-[[2-(2,4-dichlorophenyl)-4-propyl-1,3-dioxolan-2-yl]methyl]-1,2,4-triazole (CAS No. 60207-90-1)). In an aspect, propiconazole may be provided as a first fungicide treatment of a liquid system. In another aspect, propiconazole may be provided as a second fungicide treatment. In an aspect, propiconazole may be provided as a third fungicide treatment. In yet another aspect, propiconazole may be provided as fourth treatment or a fifth treatment. In another aspect, propiconazole may be provided as sixth treatment or a seventh treatment. In other embodiments, propiconazole may be administered in combination with one or more fungicides either separately by being administered contemporaneously with the one or more fungicides, or as part of a mixture of fungicides.

The present disclosure further provides for the treatment of a liquid system with the fungicide thiophanate-methyl (methyl N-[[2-(methoxycarbonylcarbamothioylamino) phenyl]carbamothioyl]carbamate (CAS No. 23564-05-8)). In an aspect, thiophanate-methyl may be provided as a first fungicide treatment of a liquid system. In another aspect, thiophanate-methyl may be provided as a second fungicide treatment. In an aspect, thiophanate-methyl may be provided as a third fungicide treatment. In yet another aspect, thiophanate-methyl may be provided as fourth treatment or a fifth treatment. In another aspect, thiophanate-methyl may be provided as sixth treatment or a seventh treatment. In other embodiments, thiophanate-methyl may be administered in combination with one or more fungicides either separately by being administered contemporaneously with the one or more fungicides, or as part of a mixture of fungicides.

The present disclosure further provides for the treatment of a liquid system with the fungicide tolylfluanid (N-[dichloro(fluoro)methyl]sulfanyl-N-(dimethylsulfamoyl)-4-methylaniline (CAS No. 731-27-1)). In an aspect, tolylfluanid may be provided as a first fungicide treatment of a liquid system. In another aspect tolylfluanid may be provided as a second fungicide treatment. In an aspect, tolylfluanid may be provided as a third fungicide treatment. In yet another aspect, tolylfluanid may be provided as fourth treatment or a fifth treatment. In another aspect, tolylfluanid may be provided as sixth treatment or a seventh treatment. In other embodiments, tolylfluanid may be administered in combination with one or more fungicides either separately by being administered contemporaneously with the one or more fungicides, or as part of a mixture of fungicides.

The present disclosure further provides for the treatment of a liquid system with the fungicide triadimenol A (1-(4-Chlorophenoxy)-3,3-Dimethyl-1-(1,2,4-Triazol-1-yl)-Butanol (CAS No. 89482-17-7)). In an aspect, triadimenol A may be provided as a first fungicide treatment of a liquid system. In another aspect triadimenol A may be provided as a second fungicide treatment. In an aspect, triadimenol A may be provided as a third fungicide treatment. In yet another aspect, triadimenol A may be provided as fourth treatment or a fifth treatment. In another aspect, triadimenol A may be provided as sixth treatment or a seventh treatment. In other embodiments, triadimenol may be administered in combination with one or more fungicides either separately by being administered contemporaneously with the one or more fungicides, or as part of a mixture of fungicides.

The present disclosure further provides for the treatment of a liquid system with a fungicide of Table 1, but not including the fungicides azoxystrobin, binapacryl, boscalid, captan, cyazofamid, cymoxanil, dimoxystrobin, dinocap, dodemorph, endothal monohydrate, fenhexamid, fosetyl-aluminum (100 mg), kresoxim-methyl, mancozeb, metalaxyl, pencycuron, propamocarb, prothioconazole, pyrifenox, sonar, spiroxamine, tebuconazole, trifloxystrobin, triflumizole, triforin, and zoxamide.

In another aspect, the treatment methods provide for excluding the fungicides amphotericin b trihydrate, malachite green, diiodine/iodopentoxide, sodium percarbonate, TCC acid, hymexazol and octhilinone due to known toxic effects and health hazards.

In further aspect, fluazinam may be provided alone, or in combination with one or more fungicides of Table 1. In an aspect, fluazinam may be provided as a treatment in combination with pyraclostrobin. In an aspect, fluazinam precedes a treatment of a liquid system with pyraclostrobin. In another aspect, fluazinam treatment follows a treatment of a liquid system with pyraclostrobin. In an aspect, fluazinam precedes a treatment of a liquid system with Thiram®. In another aspect, fluazinam treatment follows a treatment of a liquid system with Thiram®. In an aspect, fluazinam precedes a treatment of a liquid system with chlorothalonil. In another aspect, fluazinam treatment follows a treatment of a liquid system with chlorothalonil. In an aspect, fluazinam precedes a treatment of a liquid system with dodine. In another aspect, fluazinam treatment follows a treatment of a liquid system with dodine.

In a further aspect, pyraclostrobin may be provided alone, or in combination with one or more fungicides of Table 1. In an aspect, pyraclostrobin may be provided as a treatment in combination with fluazinam. In an aspect, pyraclostrobin precedes a treatment of a liquid system with fluazinam. In another aspect, pyraclostrobin treatment follows a treatment of a liquid system with fluazinam. In an aspect, pyraclostrobin precedes a treatment of a liquid system with Thiram®. In another aspect, pyraclostrobin treatment follows a treatment of a liquid system with Thiram®. In an aspect, pyraclostrobin may be provided as a treatment in combination with chlorothalonil. In an aspect, pyraclostrobin precedes a treatment of a liquid system with chlorothalonil. In another aspect, pyraclostrobin treatment follows a treatment of a liquid system with chlorothalonil. In an aspect, pyraclostrobin precedes a treatment of a liquid system with dodine. In another aspect, pyraclostrobin treatment follows a treatment of a liquid system with dodine.

In further aspect, Thiram® may be provided alone, or in combination with one or more fungicides of Table 1. In an aspect, Thiram® may be provided as a treatment in combination with fluazinam. In an aspect, Thiram® precedes a treatment of a liquid system with fluazinam. In another aspect, Thiram® treatment follows a treatment of a liquid system with fluazinam. In an aspect, Thiram® precedes a treatment of a liquid system with pyraclostrobin. In another aspect, Thiram® treatment follows a treatment of a liquid system with pyraclostrobin. In an aspect, chlorothalonil may be provided as a treatment in combination with Thiram®. In an aspect, Thiram® precedes a treatment of a liquid system with chlorothalonil. In another aspect, Thiram® treatment follows a treatment of a liquid system with chlorothalonil. In an aspect, dodine may be provided as a treatment in combination with Thiram®. In an aspect, Thiram® precedes a treatment of a liquid system with dodine. In another aspect, Thiram® treatment follows a treatment of a liquid system with dodine.

In further aspect, chlorothalonil may be provided alone, or in combination with one or more fungicides of Table 1. In an aspect, chlorothalonil may be provided as a treatment in combination with fluazinam. In an aspect, chlorothalonil precedes a treatment of a liquid system with fluazinam. In an aspect, chlorothalonil may be provided as a treatment in combination with pyraclostrobin. In an aspect, chlorothalonil precedes a treatment of a liquid system with pyraclostrobin. In another aspect, chlorothalonil treatment follows a treatment of a liquid system with pyraclostrobin. In an aspect, chlorothalonil may be provided as a treatment in combination with Thiram®. In an aspect, chlorothalonil precedes a treatment of a liquid system with Thiram®. In another aspect, chlorothalonil treatment follows a treatment of a liquid system with Thiram®. In an aspect, chlorothalonil precedes a treatment of a liquid system with dodine. In another aspect, chlorothalonil treatment follows a treatment of a liquid system with dodine.

In further aspect, dodine may be provided alone, or in combination with one or more fungicides of Table 1. In an aspect, dodine may be provided as a treatment in combination with fluazinam. In an aspect, dodine precedes a treatment of a liquid system with fluazinam. In an aspect, dodine may be provided as a treatment in combination with pyraclostrobin. In an aspect, dodine precedes a treatment of a liquid system with pyraclostrobin. In another aspect, dodine treatment follows a treatment of a liquid system with pyraclostrobin. In an aspect, dodine may be provided as a treatment in combination with Thiram®. In an aspect, dodine precedes a treatment of a liquid system with Thiram®. In another aspect, dodine treatment follows a treatment of a liquid system with Thiram®.

In an aspect of the present disclosure, a combination of fluazinam, pyraclostrobin, chlorothalonil and dodine may be used to treat a liquid system. Specifically, the combinations may be provided sequentially to a liquid system over an extended period to ensure control of the pest in a culture of microalgae. In an aspect, the order of the treatment of a pest may be determined by selecting a subsequent fungicide based on a differing mode of action. In an aspect, a treatment regimen of a liquid system may be provided wherein a second fungicide does not follow a first fungicide having the same mode of action. In a further aspect, the first fungicide and the third fungicide have a different mode of action. In an aspect, rotation of fluazinam, pyraclostrobin, chlorothalonil and dodine as treatments of a liquid system for the control of pests may be used to avoid the development of resistant strains of pest.

One of skill in the art would understand that additional combinations of the fungicides of Table 1 may be selected. In an aspect, a first fungicide is selected that decreases the growth of a pest in a culture of microalgae. In another aspect, a second fungicide is selected that differs from the first fungicide in its mechanism of action. In one aspect, a first fungicide may be an inhibitor of respiration and a second fungicide may be a sterol biosynthesis inhibitor. In another aspect, a first fungicide may be an inhibitor of respiration that uncouples oxidative phosphorylation and a second fungicide may be a quinone outside inhibitor of respiration. In another aspect, a first fungicide may be an inhibitor of respiration that uncouples oxidative phosphorylation and a second fungicide may have multi site contact activity. In yet another aspect, a first fungicide may be a demethylation inhibitor and a second fungicide may have multi site contact activity. One of ordinary skill in the art would understand that selection of fungicides based on different mechanisms of action provides methods that avoid the development of fungicide resistant pest strains. Any combination of inhibitory methods of action may be combined for administration either in series or comtemporaneously.

Fungicides may be introduced by methods known in the art. In an aspect, the fungicides may be introduced as a solid. In another aspect, the fungicides may be introduced after solvation in an appropriate solvent. In an aspect, a solvent may be water. In another aspect, the fungicide may be dissolved in an alcohol. In an aspect the alcohol may be methanol. In another aspect, the alcohol may be ethanol. In an aspect, the fungicide may be prepared in acetonitrile. In yet another aspect, the fungicide may be prepared in acetone. In still another aspect the fungicide may be dissolved in the culture medium used to grow the microalgae. In an aspect, the effect of the solvent on the organism or organisms is minimized.

The present disclosure provides for the introduction of fungicides at an effective concentration. Effective concentrations may be determined according to manufacturer's instructions or may be determined empirically. An effective concentration of a fungicide is not toxic to the microalgae being cultured in the liquid system. Methods to determine toxicity are known in the art and include serial dilutions of a test fungicide in a growing liquid culture of microalgae. Fungicides begin to show growth effects on microalgae in the ranges provided in Table 2. One of skill in the art would understand that different microalgae may have different ranges of toxicity that may be determined by growth of a microalga in the presence of a serial dilution of a fungicide.

TABLE 2
Ranges of Microalgae Toxicity

| | Ranges of Microalgae |
|---|---|
| Description | toxicity (ppm) |
| benodanil | 0.3125-1.25 |
| binapacryl | 0.125-0.5 |
| captan | 1.953-125 |
| carboxine | 0.977-3.906 |
| cyazofamid | 0.03125-0.125 |
| cymoxanil | 15.625-62.5 |
| dimoxystrobin | 0.004-0.0625 |
| dinocap | 0.0039-0.0625 |
| dithianon | 0.625-2.5 |
| dodemorph | 0.195-0.781 |
| fenarimol | 0.0489-0.195 |
| fenhexamid | 15.63-62.5 |
| fenpropidin | 0.00195-1.953 |
| fluazinam | >7.5 |
| pencycuron | 0.781-12.5 |
| propamocarb | 3.906-62.5 |
| pyraclostrobin | >15 |
| pyrifenox | 0.004-0.156 |
| spiroxamine | 0.0625-1.0 |
| Thiram ® | >20 |
| tolyifluanid | 1.56-25.0 |
| triflumizole | 1.563-6.25 |
| zoxamide | 0.0156-0.250 |

According to the present disclosure, a fungicide may be toxic to a microalgae if the growth of a microalgae is decreased in a given concentration range. In an aspect, an effective concentration of fungicide may cause a decrease in microalgae growth but causes a greater reduction in the growth of a pest.

The present disclosure provides for effectiveness to be expressed as a ratio of the decrease in growth of a pest to the decrease in growth of a microalga. In an aspect, the growth of a pest may be reduced by 10 fold (e.g., 0.1×) relative to the growth in the absence of a fungicide and the growth of a microalgae decreased by 50% (e.g., 0.5×) relative to the growth in the absence of a fungicide to provide an effectiveness ratio of 0.2. In another aspect, the growth of a pest may be reduced by 10 fold and the microalgae decreased by 20% (e.g., 0.8×) to result in an effectiveness ratio of 0.125. In an aspect, an effectiveness ratio may be less than 0.8. In another aspect, an effectiveness ratio may be less than 0.4. In another aspect, an effectiveness ratio may be less than 0.2. In another aspect, an effectiveness ratio may be less than 0.1. In another aspect, an effectiveness ratio may be less than 0.05.

In another aspect, the effectiveness is expressed as a useful therapeutic window. A useful therapeutic window is defined as the difference in the impact of the fungicide on the algae versus the pest. In an aspect, a useful therapeutic window is the difference between the concentration of fungicide that impacts microalgae growth and the concentration that impacts pest growth (e.g., concentration of fungicide that impacts microalgae growth minus the concentration that impacts pest growth). In an aspect, the growth rate of a microalga starts to be impacted at 2 ppm, and growth of pests is impacted at 0.5 ppm to provide a therapeutic window of 1.5 ppm. In an aspect, the therapeutic window may be 1 ppm. In an aspect the therapeutic window may be 1.5 or 2.0 ppm. In another aspect, the therapeutic window may be greater than 0.5 ppm. In another aspect, the therapeutic window may be greater than 1.0 ppm. In yet another aspect, the therapeutic window may be greater than 1.5 ppm. In another aspect, the therapeutic window may be greater than 2.0 ppm. In another aspect, the therapeutic window may be greater than 2.5 ppm. In another aspect, the therapeutic window may be greater than 5.0 ppm.

In a further aspect, the therapeutic window may be from 0.5 to 1.0 ppm. In another aspect, the therapeutic window may be from 0.5 to 1.5 ppm. In another aspect, the therapeutic window may be from 0.5 to 2.0 ppm. In an aspect, the therapeutic window may be from 0.5 to 2.5 ppm. In an aspect, the therapeutic window may be from 0.5 to 5.0 ppm. In another aspect, the therapeutic window may be from 1.0 to 1.5 ppm. In another aspect, the therapeutic window may be from 1.0 to 2.0 ppm. In an aspect, the therapeutic window may be from 1.0 to 2.5 ppm. In an aspect, the therapeutic window may be from 1.0 to 5.0 ppm. In another aspect, the therapeutic window may be from 1.5 to 2.0 ppm. In an aspect, the therapeutic window may be from 1.5 to 2.5 ppm. In an aspect, the therapeutic window may be from 1.5 to 5.0 ppm. In an aspect, the therapeutic window may be from 2.0 to 2.5 ppm. In an aspect, the therapeutic window may be from 2.0 to 5.0 ppm.

In yet another aspect, the effectiveness of the fungicide provides for a negative therapeutic window. For example, where the growth rate of algae is impacted at 2 ppm and the growth rate of pests are impacted at 2.5 ppm a negative therapeutic window is −0.5 ppm. Fungicides with a negative therapeutic window are generally not considered effective. However, in an aspect, decreased microalgae growth may be provided for where the integrated growth rate is greater than zero. A decreased microlagae growth rate may be acceptable for 1 or 2 days. In another aspect, a decreased microalgae growth rate may be acceptable for 3 days. In another aspect, a decreased microalgae growth rate may be acceptable for 4 days. In another aspect, a decreased microalgae growth rate may be acceptable for less than 1 week.

The present disclosure further provides for effectiveness to be expressed as a percent growth rate of a treated culture over an uninfected control growth rate (the “percent efficacy”). In an aspect, an effective fungicide may have a percent efficacy between 90 and 100%. In another aspect, an effective fungicide may have a percent efficacy between 80 and 100%. In an aspect, an effective fungicide may have a percent efficacy between 76 and 100%. In an aspect, an effective fungicide may have a percent efficacy of 76% or greater. In another aspect, an effective fungicide may have a percent efficacy of 80° % or greater. In an aspect, an effective fungicide may have a percent efficacy of 90% or greater.

In an aspect, an effective fungicide may have a percent efficacy between 51 and 75%. In another aspect, an effective fungicide may have a percent efficacy between 60 and 75%. In another aspect, an effective fungicide may have a percent efficacy between 65 and 75%. In yet another aspect, an effective fungicide may have a percent efficacy between 26 and 50%. In an aspect an effective fungicide may have a percent efficacy between 30 and 50%. In an aspect an effective fungicide may have a percent efficacy between 40 and 50%. In an aspect, an effective fungicide may have a percent efficacy of 51% or greater. In another aspect, an effective fungicide may have a percent efficacy 60% or greater. In an aspect, an effective fungicide may have a percent efficacy of 70% or greater.

In an aspect, an effective concentration of fluazinam may be 0.5 ppm, or less. In another aspect an effective concentration of fluazinam may be 1.0 ppm, or less. In an aspect an effective concentration of fluazinam may be 2.0 ppm, or less. In a further aspect, an effective concentration of fluazinam may be 5.0 ppm, or less. In another aspect an effective concentration of fluazinam may be 10.0 ppm, or less. In another aspect an effective concentration of fluazinam may be more than 10.0 ppm. In an aspect, an effective concentration of fluazinam provides for a percent efficacy of between 51 and 75%. In another aspect, an effective concentration of fluazinam provides for a percent efficacy of greater than 50%.

In one aspect, an effective concentration of fluazinam may range from 0.1 to 0.5 ppm. In another aspect, an effective concentration of fluazinam may be a range from 0.5 to 1 ppm. In an aspect, an effective concentration of fluazinam may be from 0.5 to 2 ppm. In an aspect, an effective concentration of fluazinam may be from 0.5 to 5 ppm. In an aspect, an effective concentration of fluazinam may be from 0.5 to 10 ppm. In further aspect, an effective concentration of fluazinam may be from 1 to 2 ppm. In an aspect, an effective concentration of fluazinam may be from 1 to 5 ppm. In an aspect, an effective concentration of fluazinam may be from 1 to 10 ppm. In further aspect, an effective concentration of fluazinam may be from 2 to 5 ppm. In an aspect, an effective concentration of fluazinam may be from 2 to 10 ppm. In yet another aspect, an effective concentration of fluazinam may be from 5 to 10 ppm.

In an aspect, an effective concentration of pyraclostrobin may be 0.5 ppm, or less.

In another aspect an effective concentration of pyraclostrobin may be 1.0 ppm, or less. In an aspect an effective concentration of pyraclostrobin may be 2.0 ppm, or less. In a further aspect, an effective concentration of pyraclostrobin may be 5.0 ppm, or less. In another aspect an effective pyraclostrobin of fluazinam may be 10.0 ppm, or less. In another aspect an effective concentration of pyraclostrobin may be more than 10.0 ppm. In an aspect, an effective concentration of pyraclostrobin provides for a percent efficacy of between 51 and 75%. In another aspect, an effective concentration of pyraclostrobin provides for a percent efficacy of greater than 50%.

In one aspect, an effective concentration of pyraclostrobin may range from 0.1 to 0.5 ppm. In another aspect, an effective concentration of pyraclostrobin may be a range from 0.5 to 1 ppm. In an aspect, an effective concentration of pyraclostrobin may be from 0.5 to 2 ppm. In an aspect, an effective concentration of pyraclostrobin may be from 0.5 to 5 ppm. In an aspect, an effective concentration of pyraclostrobin may be from 0.5 to 10 ppm. In further aspect, an effective concentration of pyraclostrobin may be from 1 to 2 ppm. In an aspect, an effective concentration of pyraclostrobin may be from 1 to 5 ppm. In an aspect, an effective concentration of pyraclostrobin may be from 1 to 10 ppm. In further aspect, an effective concentration of pyraclostrobin may be from 2 to 5 ppm. In an aspect, an effective concentration of pyraclostrobin may be from 2 to 10 ppm. In yet another aspect, an effective concentration of pyraclostrobin may be from 5 to 10 ppm.

In an aspect, an effective concentration of Thiram® may be 0.5 ppm, or less. In another aspect an effective concentration of Thiram® may be 1.0 ppm, or less. In an aspect an effective concentration of Thiram® may be 2.0 ppm, or less. In a further aspect, an effective concentration of Thiram® may be 5.0 ppm, or less. In another aspect an effective concentration of Thiram® may be 10.0 ppm, or less. In another aspect an effective concentration of Thiram® may be more than 10.0 ppm. In an aspect, an effective concentration of Thiram® provides for a percent efficacy of between 26 and 50%. In another aspect, an effective concentration of Thiram® provides for a percent efficacy of greater than 26%.

In one aspect, an effective concentration of Thiram® may range from 0.1 to 0.5 ppm. In another aspect, an effective concentration of Thiram® may be a range from 0.5 to 1 ppm. In an aspect, an effective concentration of Thiram® may be from 0.5 to 2 ppm. In an aspect, an effective concentration of Thiram® may be from 0.5 to 5 ppm. In an aspect, an effective concentration of Thiram® may be from 0.5 to 10 ppm. In further aspect, an effective concentration of Thiram® may be from 1 to 2 ppm. In an aspect, an effective concentration of Thiram® may be from 1 to 5 ppm. In an aspect, an effective concentration of Thiram® may be from 1 to 10 ppm. In further aspect, an effective concentration of Thiram® may be from 2 to 5 ppm. In an aspect, an effective concentration of Thiram® may be from 2 to 10 ppm. In yet another aspect, an effective concentration of Thiram® may be from 5 to 10 ppm.

Methods of the present disclosure provide for increasing the yield of harvested microalgae. In an aspect, the methods provide for an increased yield of harvested microalgae in a liquid system compared to the yield of microalgae in the absence of providing an effective concentration of fungicide or pesticide. One aspect provides a yield of microalgae greater than 0.4 gram per liter (g/l) AFDW (Ash Free Dry Weight).

Yields can be determined by the number of microorganisms per volume of liquid culture. Yields may be increased by increasing the total culture volume or by optimizing the density of microalgae. Methods of the present disclosure provide for increased density of microalgae. In an aspect, the yield at harvest following growth of microalgae in the liquid culture system may be less than the growth of the microalgae in the absence of fungicide treatment in the absence of a pest, but greater than the yield provided in the presence of a pest without the fungicide treatment.

In an aspect, a yield is greater than 0.5 g/l after fungicide treatment. In another aspect, the yield is greater than 0.6 or greater than 0.7 g/l. In a further aspect, the yield of microalgae is greater than 0.8 or greater than 0.9 g/l. In yet a further aspect, the yield of microalgae may be greater than 1.0 g/l.

In an aspect, a yield of microalgae is at least 80% of the yield of microalgae harvested from an uninfected liquid culture of microalgae that has not been provided a fungicide. In another aspect, a yield is at least 85% or at least 90% of a yield of microalgae harvested from an uninfected liquid culture of microalgae that has not been provided a fungicide. In another aspect, a yield is at least 95% or at least 97.5% of a yield of microalgae harvested from an uninfected liquid culture of microalgae that has not been provided a fungicide. In a further aspect, a yield is at least 99% or 100% of the yield of microalgae harvested from an uninfected liquid culture of microalgae that has not been provided a fungicide.

In a further aspect, a yield of microalgae is at least 10% greater than the yield of microalgae harvested from a liquid culture of microalgae having a pest and that has not been provided a fungicide. In an aspect, the yield of microalgae harvested from a liquid culture of microalgae having a pest and that has not been provided a fungicide is at least 15% or at least 20% greater. In an aspect, the yield of microalgae harvested from a liquid culture of microalgae having a pest and that has not been provided a fungicide is at least 25% greater. In another aspect, the yield of microalgae harvested from a liquid culture of microalgae having a pest and that has not been provided a fungicide is at least 50% greater. In another aspect, the yield of microalgae harvested from a liquid culture of microalgae having a pest and that has not been provided a fungicide is at least 75% greater. In another aspect, the yield of microalgae harvested from a liquid culture of microalgae having a pest and that has not been provided a fungicide is at least 100% greater. In an aspect, the greater yield may not be determined where the untreated liquid culture would not survive absent a fungicide treatment.

In a further aspect, the yield may be 1.5 fold higher than the yield of microalgae harvested from a liquid culture of microalgae having a pest and that has not been provided a fungicide. In another aspect, the yield of microalgae harvested from a liquid culture of microalgae having a pest and that has not been provided a fungicide is at least 2.0 fold greater. In another aspect, the yield of microalgae harvested from a liquid culture of microalgae having a pest and that has not been provided a fungicide is at least 2.5 or 5.0 fold greater. In another aspect, the yield of microalgae harvested from a liquid culture of microalgae having a pest and that has not been provided a fungicide is at least 7.5 fold greater. In an aspect the yield may be at least 10 fold greater in the fungicide treated liquid culture than an untreated liquid culture having a pest. In an aspect, the increased yield may be 15 fold or even greater than the yield of microalgae harvested from a liquid culture of microalgae having a pest and that has not been provided a fungicide.

The present disclosure provides for the detection of a pest in a liquid culture of microalgae by periodic monitoring. In an aspect, the monitoring may be performed daily. In a further aspect, the monitoring may be performed twice daily. In yet a further aspect, the monitoring may be performed three or more times each day. In a further aspect of the invention, the monitoring may be conducted every other day. In yet another aspect, the monitoring may be performed weekly.

In an aspect, the monitoring may be performed at a specified time of the day. In an aspect, the monitoring may be conducted in the morning. In another aspect, the monitoring may be conducted at mid-day. In yet another aspect, the monitoring may be performed at or near sunset. In another aspect, monitoring may be performed at night. In an aspect, monitoring may be performed at two periods each day, for example in the morning and again in the evening. In another aspect, monitoring may occur during the day and a second monitoring may occur at night.

In a further aspect, the monitoring may be done continuously. In an aspect, the continuous monitoring may be done by using a continuous flow assay, for example a FlowCAM® (Fluid Imaging Technologies, Yarmouth, Me.). FlowCAM analysis integrates flow cytometry and microscopy allowing for high-throughput analysis of particles in a moving field. Diluted (1:10) culture samples are run through the FlowCAM with a 20× objective (green algae) or a 4× objective (blue-green algae). The FlowCAM and its integrated software automatically images, counts, and analyzes a predetermined amount of particles (typically 3,000) in a continuous flow. Libraries are then constructed allowing particles to be sorted by various phenotypic attributes (e.g. green vs. transparent cells, large cells vs. small cells, etc). Particle sorting can also be customized to specifically identify organisms of interest.

In an aspect, the monitoring may detect a change in fluorescence of a culture of microalgae in a liquid system. In an aspect, the growth of microalgae in a liquid system may be monitored by detecting chlorophyll fluorescence. Measurement of the natural fluorescence of chlorophyll provides a measurement of growth and, in an aspect, provides greater sensitivity than growth monitoring by light scattering, particularly in the presense of non-photosynthetic co-occurring organisms. In another aspect, a ratio of fluorescence may be detected using an excitation wavelength of 488 and determining the peak of an emission spectra at different wavelengths. In an aspect, the peak of the emission spectra is greatest between the wavelengths of 710 nm and 688 nm. If the excitation emission data decreases over time, this is indicative of the presence of an infection.

In another aspect, the fluorescence of a culture may be determined using an excitation wavelength of 360 nm and measuring the emission at 440 nm, 530 nm, 685 nm or 740 nm. Changes in the ratios of the emissions at these wavelengths are known to one of skill in the art to be indicative of stress.

In an aspect, chlorophyll fluorescence in a desmid culture may be measured using an excitation wavelength of 430 nm and an emission wavelength of 685 nm. In another aspect, Spirulina growth may be monitored by chlorophyll fluorescence using an excitation wavelength of 363 nm and an emission wavelength of 685 nm. The results of microalgae growth may be used to prepare a semi-log plot of chlorophyll fluorescence versus time. Such graphs provide a growth curve.

In yet another aspect, the pond may be monitored using a fluorescent dye binding assay. In fluorescent dye binding assays, the amount of fluorescent dye bound by microalgae is increased by the presence of an infection. In an aspect, the dye may bind to glucans found in cellulose. In an aspect, the glucan may be chitin that may be found in fungal cell walls. In an aspect, the fluorescent dye may be Calcofluor White (Sigma, Cat. #18909). In another aspect, the dye may be Solaphenyl flavine (Aakash Chemicals, Solophenyl Flavine 7GFE). Increased binding of Calcofluor White and Solaphenyl flavine corresponds to the binding of the dye to cell wall contaminants not present in non-infected cultures of microalgae. Additional dye binding assays may be developed for any dye that binds with low affinity to a microalga and binds with high affinity to a pest, for example, a chytrid.

In an aspect, the binding of a 1% solution of Calcofluor White (Sigma, Cat. #18909) is detected by measuring fluorescence with an excitation wavelength of 360 nm and emission detected at 444 nm. In an aspect, Calcofluor White treated samples may be examined microscopically using a DAPI filter. In yet another aspect, samples may be monitored for fungal contamination using Solaphenyl flavine fluorescent dye binding. Solaphenyl flavine staining may be measured using an excitation wavelength of 365 nm and emission wavelength of 515 nm. In an aspect microscopic examination of a sample binding Solaphenyl flavine fluorescent dye may be performed using a FITC filter.

In another aspect, the monitoring may detect a change in light scattering, for example the absorption of light at 795 nm. Methods for continuously monitoring the growth of a microalgae are known in the art, for example in Sode et al., “On-line monitoring of marine cyanobacterial cultivation based on phycocyanin fluorescence,” J. Biotechnology 21:209-217 (1991), Torzillo et al., “On-Line Monitoring of Chlorophyll Fluorescence to Assess the Extent of Photoinhibition of Photosynthesis Induced by High Oxygen Concentration and Low Temperature and its Effect on the Productivity of Outdoor Cultures of Spirulina Platensis (Cyanobacteria),” J. Phycology 34:504-510 (1998), and Jung and Lee “In Situ Monitoring of Cell Concentration in a Photobioreactor Using Image Analysis: Comparison of Uniform Light Distribution Model and Artificial Neural Networks” Biotechnology Progress 22:1443-1450 (2006), each of which are herein incorporated by reference in their entireties.

Microalgae ponds may be monitored using a flocculation assay. In an aspect, flocculation may be measured by determining the ratio of microalgae remaining after a defined time period. A sample may contain the amount of suspended microalgae determined by light scattering or fluorescence as provided above (e.g., T₀). After a period, a second determination may be made (e.g., Tₙ) and the ratio determined (e.g., Tₙ/T₀). In an aspect, the ratio of may be determined at 40 minutes (e.g., T₄₀/T₀). In another aspect, the ratio may be determined at 30 or 60 minutes. In a further aspect, multiple time points may be obtained and the flocculation expressed as a slope of the amount of algae in suspension versus time.

In accordance with the present disclosure, detection of a pest in the liquid system indicates a need for providing an effective concentration of a fungicide or pesticide to inhibit the growth of a pest. In an aspect, a change in the outcome of a test compared to the prior test may indicate a need for an additional test. In another aspect, a positive test result may indicate a need for an additional test with greater sensitivity.

In an aspect, detection of a pest in the liquid system may detect one or more pests. In an aspect, two or more tests for a pest may be performed. In another aspect, three or more tests are performed. In a further aspect, 4 or more or even 5 or more tests are performed. In an aspect, between 1 and 5 tests are performed. In an aspect, the number of tests performed is determined by the microalgae. In an aspect, test for the pests of the genera Scenedesmus, Desmodesmus, Nannochloropsis and Spirulina are performed.

In an aspect, pests may be detected using Polymerase Chain Reaction (PCR) to detect ribosomal sequences. In an aspect, ribosomal sequences may include DNA sequence selected from the group consisting of NC_{—}003053 Rhizophydium sp. 136 mitochondrion, NC_{—}003048 Hyaloraphidium curvatum mitochondrion. NC_{—}003052 Spizellomyces punctatus mitochondrion chromosome 1, NC_{—}003061 Spizellomyces punctatus mitochondrion chromosome 2, NC_{—}003060 Spizellomyces punctatus mitochondrion chromosome 3, NC_{—}004760 Harpochytrium sp. JEL94 mitochondrion, NC_{—}004624 Monoblepharella sp. JEL15 mitochondrion, and NC_{—}004623 Harpochytrium sp. JEL105 mitochondrion. In another aspect of the present disclosure, pests may be detected using PCR that amplifies a sequence selected from SEQ ID NOs: 1 to 6.

Methods of the present disclosure include methods of detection that may detect a pest present at a level of at least 10⁵ cells/ml. In another aspect, the methods of the present disclosure provide for the detection of a pest at a concentration 10⁴ cells/ml. In a further aspect, the concentration of pest may be detected at 10³ cells/ml. In another aspect, a pest present at a concentration of 10² cells/ml or even 10¹ cells/ml may be detected.

The polymerase chain reaction (PCR) is a sensitive method for the detection of the presence of an organism in a sample. Methods for performing PCR are known in the art. Nucleic acid analysis by PCR requires sample preparation, amplification, and product analysis. Although these steps are usually performed sequentially, amplification and analysis can occur simultaneously. Quantitative analysis occurs concurrently with amplification in the same tube within the same instrument. The concept of combining amplification with product analysis has become known as “real time’ PCR or quantitative PCR (qPCR). See, for example, U.S. Pat. No. 6,174,670, herein incorporated by reference in its entirety.

In an aspect, real-time methods of PCR may be used to detect the presence of a pest in a liquid system (e.g., quantitative PCR). In a real time PCR assay, a fluorescent signal accumulates during each amplification cycle. A positive reaction is provided when the fluorescent signal exceeds a threshold level, typically the background fluorescence. The cycle threshold (Cₜ) the number of cycles required to cross the threshold and the Cₜ levels are inversely proportional to the amount of target nucleic acid in the sample (i.e., the lower the Cₜ level the greater the amount of target nucleic acid in the sample). Real time PCR assays typically undergo 40 cycles of amplification. A person of ordinary skill would recognize that the Cₜ value may be compared to a standard curve prepared from a serially diluted pest to determine a number of pests/ml of sample.

In an aspect, a pest is detected when the Cₜ value is less than 35 cycles for at least one monitoring step. In another aspect, a pest is detected when the Cₜ value is less that 35 cycles for at least two consecutive monitoring steps. In yet another aspect, a Cₜ value of less than 35 cycles for three consecutive monitoring steps indicates the presence of a pest.

The present disclosure further provides for the detection of pest when there is a consistent decrease in the Cₜ over two or more monitoring steps. In an aspect, a consistent decrease from a Cₜ of 35 or higher to a Cₜ value of 30 or less indicates a need for crop protective action. In an aspect, a Cₜ of less than 30 for chytrid pest identifiable using SEQ ID NO: 1 indicates a need for crop protective action. In another aspect, a Cₜ of less than 30 for chytrid pest identifiable using SEQ ID NO: 2 indicates a need for crop protective action.

The present disclosure provides for the detection of pest using fluorescence. In an aspect, a pest is detected when the average percentage change of chlorophyll fluorescence is negative over a three day period.

The present disclosure further provides for the continued monitoring and detection of pests in a liquid system after detection of a pest contamination and after providing an effective concentration of a pesticide or fungicide. The present disclosure provides for continued monitoring to determine the effectiveness of treatment as well as for the detection of subsequent pest contamination of the liquid system.

The present disclosure provides for the collection and processing of samples for monitoring of a liquid system. Samples may be collected under any one or more monitoring regimens of the claimed invention. Depending on the size of the liquid system, samples may be collected randomly or systematically. In an aspect, samples may be collected from a single location. In another aspect, samples may be collected from multiple locations. In an aspect, multiple samples may be pooled and analyzed. In another aspect, multiple samples may be analyzed separately. Statistical methods known to those of skill in the art may be applied to sample collection and analysis. See, e.g., Biometry: The Principles and Practices of Statistics in Biological Research. Robert R. Sokal, F. James Rohlf. W. H. Freeman. 1994.

Samples collected according the methods of the present disclosure may be processed for further analysis. In an aspect, the DNA of a sample may be extracted according methods known in the art. In an aspect, DNA may be obtained for further analysis by boiling the sample in a sodium dodecyl sulfate (SDS) containing buffer. In another aspect, sample DNA may be obtained by ‘bead beating’ the sample followed by centrifugation. In yet another aspect, DNA for analysis may be obtained from lysed samples by absorption and elution from a solid phase, for example using kits known in the art. Non-limiting examples of DNA extraction methods may be found, for example in “Current Protocols in Molecular Biology” Volumes 1 and 2, Ausubel F. M. et al., published by Greene Publishing Associates and Wiley Interscience (1989) or in Molecular Cloning, T. Maniatis. E. F. Fritsch, J. Sambrook, 1982, or in Sambrook J. and Russell D., 2001, Molecular Cloning: a laboratory manual (Third edition), each of which are incorporated herein in there entireties.

The present disclosure provides for the treatment of a liquid culture with an effective concentration of a pesticide or fungicide. The ongoing monitoring provides for the information necessary for one of ordinary skill to make the decision to treat the liquid system as well as determine which of the treatments of the present disclosure to apply. In an aspect, rapid treatment at an indication of pest contamination provides for a maximal enhancement of microalgae yield. In an aspect, failure to treat upon detection of a pest may result in the collapse and loss of the microalgae culture in the liquid system. In another aspect, delay in treatment may result in decreased yields of microalgae in the liquid system.

The present disclosure provides for the treatment of a liquid culture with an effective concentration of a pesticide or fungicide when the threshold cycle for a pest detected by qPCR(Cₜ) is below 30. In an aspect, the need for a treatment is indicated when the Cₜ is below 29. In another aspect, the need for a treatment is indicated when the Cₜ is below 28.

In an aspect, treatment of a liquid culture is indicated when there is a decrease in chlorophyll fluorescence. In an aspect, if the average chlorophyll fluorescence does not increase over three days, a treatment with an effective concentration of pesticide or fungicide is indicated. In an aspect, if the percentage change in average chlorophyll fluorescence does not increase over three days, a treatment with an effective concentration of pesticide or fungicide is indicated. In an aspect, if the percentage change in average chlorophyll fluorescence decreases over three days, a treatment with an effective concentration of pesticide or fungicide is indicated. In another aspect, if the percentage change in average chlorophyll fluorescence decreases by more than 5% each over two days, a treatment with an effective concentration of pesticide or fungicide is indicated.

In an aspect, the ratio of fluorescent dye binding to chlorophyll provides an indication that a treatment of a liquid culture is necessary. In an aspect, the fluorescent dye may be Caclofluor White. In another aspect the fluorescent dye may be Solaphenyl flavine. In an aspect, when the ratio of dye fluorescence to chlorophyll fluorescence is about 1.0, treatment is indicated. In another aspect, when the ratio of dye fluorescence to chlorophyll fluorescence is 1.0 or less, treatment is indicated. In an aspect, treatment is indicated when the ratio of dye fluorescence to chlorophyll fluorescence is 0.9 or less. In an aspect, when the ratio of dye fluorescence to chlorophyll fluorescence is 0.8 or less, treatment is indicated. In an aspect, treatment is indicated when the ratio of dye fluorescence to chlorophyll fluorescence is 0.7 or less. In yet another aspect, when the dye ratio is less than 0.6, treatment of a liquid culture with an effective concentration of pesticide or fungicide is indicated.

In an aspect, treatment may be provided to the liquid system within hours of the detection of a pest contamination. In an aspect, treatment may be provided within 2 hours of detection of a pest contamination. In another aspect, treatment may be provided within 4 hours of detection of a pest contamination. In yet another aspect, treatment may be provided within 8 hours of the detection of a need for crop protective action. In a further aspect, treatment may be provided within one day of detection of a need for crop protective action. In another aspect, treatment may be provided within 2 days of a need for crop protective action. In an aspect, monitoring and detection of pests may be continuous.

The present disclosure provides for continued monitoring of the liquid system and provides for subsequent treatments when detection of a pest indicates a need for crop protective action. According to the methods of the present disclosure, a liquid system may be treated two or more times upon an indication of a need for crop protective action. In another aspect, a liquid system in need of crop protective action may be treated 3 or more, or 4 or more times. In an aspect, a continuous liquid system may be treated an indefinite number of times following an indication of a need for crop protective action.

In an aspect, a subsequent treatment may be provided 5 days after a previous treatment. In another aspect, a subsequent treatment may be provided 7 days after a previous treatment. In yet another aspect, a subsequent treatment may be provided 10 or 14 days after a previous treatment. In an aspect, subsequent treatments may be provided on a bi-weekly basis.

The present disclosure also provides for subsequent treatments upon an indication of a need for crop protective action at any time following a first or subsequent treatment of an effective concentration of a pesticide or fungicide. As provided in the present disclosure, monitoring of the liquid culture and detection of a pest contamination signals the need for crop protective action. In the absence of a need for crop protective action, treatment is not necessary and growth of microalgae in a liquid system may continue for a number of weeks before a positive test for a pest indicates a need for crop protective action. As provided in the present disclosure, pesticides and fungicides may be rotated on a regular or irregular basis to prevent the development of pesticide or fungicide resistance.

Having now generally described the invention, the same will be more readily understood through reference to the following examples that are provided by way of illustration, and are not intended to be limiting of the present invention, unless specified.

Each periodical, patent, and other document or reference cited herein is hereby incorporated by reference in their entireties.

### Example 1

### Pest Identification

a. Pest Isolation

Pests are isolated using a variety of techniques. For this purpose, their designation as pests is validated after fulfilling Koch's postulates. Specifically, first a pest is found in abundance in all ponds suffering from reduced growth and is absent in a detectable manner (sustained Cₜ values less than 35) from healthy ponds. Second, a pest is isolated from an infected pond and grown in pure culture. Third, the introduction of a cultured pest causes reduced growth when introduced in a healthy experimental pond. Finally, a pest is re-isolated from the infected experimental pond and confirmed as being identical to an original pest isolated from an original pond. A number of pests have been isolated and confirmed as pests in this manner. For microalgae that class in the Spearophaeles clade, chytrids are a common pest.

b. Sample Preparation I: Boiling Method

For a limited and number of samples, a lysis buffer boiling extraction is performed. 50 μl of environmental sample is mixed with 50 μl of 0.25× lysis buffer (1×=50 mM Tris-HCl, pH 8.0; 200 mM NaCl; 20 mM EDTA, pH 8.0; 1.0% (v/v) SDS) in a 96 well Polymerase Chain Reaction (PCR) plate. The lysis buffer-sample mixture is placed into a PCR block and heated to 95° C. for 10 minutes, cooled to 25° C. for 5 minutes, heated to 95° C. for 10 minutes and then cooled to 25° C. for 5 minutes. This method extracts DNA efficiently for most microalgae pests. Efficiency is determined by the amount of DNA extracted over a dilution of template.

c. Sample Preparation II: Bead Beating Method

200 μl of sample is centrifuged at 3,500 rpm in an Eppendorf centrifuge (Model 5424) for 5 minutes and the supernatant removed. The pellet is resuspended in 200 μl of 0.25×DNA lysis buffer (1×=50 mM Tris-HCl, pH 8.0; 200 mM NaCl; 20 mM EDTA, pH 8.0; 1.0% (v/v) SDS), and lysed by a 3 min bead beating treatment in the presence of 200 μl of 0.7 mm zirconia beads (BioSpec, 11079110zx). The lysed sample is centrifuged again at 3,500 rpm for 5 min. Clear lysate is transferred to a clean tube.

d. Sample Preparation III: Norgen Plant/Fungi DNA Isolation Kit Extraction (Norgen Biotek Corp. Catalog No. 26200)).

500 μl of sample is centrifuged at 3,500 rpm in an Eppendorf centrifuge (model 5424) for 5 minutes and the supernatant is removed. The sample pellet is then lysed by a 3 minute bead beating with 400 μl of 0.7 mm zirconia beads in 400 μl of the lysis solution provided with the kit. DNA is extracted following the Norgen kit manufacturer's protocol.

e. Sample Preparation IV: MagMAX DNA Multi-Sample Kit Extraction (Applied Biosystems)

500 μl of sample is centrifuged at 3,500 rpm in an Eppendorf centrifuge (model 5424) for 5 minutes and the supernatant is removed. The sample pellet is then lysed by a 3 minute bead beating with 200 μl of 0.7 mm zirconia beads in 200 μl of Multi-Sample DNA Lysis Buffer provided with the kit. DNA is extracted following the AB kit manufacturer's protocol for isolation of Genomic DNA from cultured cells.

f. Pest Sequence Identification

A pest isolated in step a above is characterized by sequencing the Internal Transcribed Spacer 1 (ITS1) region, the 5.8S ribosomal RNA, and the Internal Transcribed Spacer 2 (ITS2) region. DNA is extracted from an isolated sample. A pest is isolated from non-axenic cultures as many pests are obligately parasitic by either plaquing or micromanipulation and are co-cultured with their hosts (e.g., the source microalgae culture). DNA from this bi-culture is amplified using the primers presented in Table 3 described below and a peptide nucleic acid (PNA) which prevents the host DNA from being amplified. PNA's include peptide nucleic acids having the sequence of SEQ ID NOs: 7 to 9. The ITS1, ITS2 region distinguishes closely related organisms but does not provide meaningful phylogenetic information. To determine evolutionary relationship of the organisms and determine its phylogenetic clade, the 18S, 5.8S, 28S regions are sequenced. These are typically concatenated and phylogenetic trees are generated. Sequences for the amplification of the ribosomal regions are presented in Table 3.

g. Polymerase Chain Reaction (PCR) Conditions

Primers used in all PCR's for sequencing in the present examples are summarized in Table 3. PCR reactions (50 μL each) are prepared in a 96-well plate as follows: 10.0 μL 5×HF buffer (New England Biolabs (NEB)), Phusion kit catalog E0553); 2.0 μL 10 mM dNTPs (NEB, Catalog E0553); 2.0 μL DMSO (Phusion kit); 5.0 μL 5M Betaine; 2.5 μL 10 μM of each primer; 2.5 μL Peptide Nucleic Acid (10 μM). If Peptide Nucleic Acids (PNAs) are in the reaction mix, a 70° C. step for 30 seconds (PNA annealing) is included in the PCR program before the 53° C. primer annealing step; 0.4 μL Phusion polymerase; 4.0 μL DNA template (prepared as described above in steps b to e), boiled and diluted 1:20 in molecular grade water (Invitrogen, 10977-015); molecular grade water (Invitrogen, 10977-015) is added to bring the total volume to 50 μL. The PCR reaction is run with the following protocol: 98° C. for 30 seconds, 40 cycles: denature at 98° C. for 10 seconds, anneal at 53° C. for 30 seconds, elongate at 72° C. for thirty seconds, the reaction is extended at 72° C. for 5 minutes, and held at 4° C. till used.

h. TOPO Cloning

Products of the PCR reaction of step g are cloned by TOPO cloning (Invitrogen Zero Blunt TOPO for Sequencing). A reaction containing 4.0 μL PCR product; 1.0 μL Salt Solution (provided with the kit); and 1.0 μL TOPO vector is prepared and incubated at room temperature for 10-30 min. While the reaction is incubating, one vial of TOP10 competent cells (Invitrogen) is thawed on ice per TOPO cloning reaction. At the end of the 10 to 30 minute incubation period, 2 μL of the TOPO cloning reaction is added to the vial of competent cells and mixed by flicking for transformation. The cells are returned to the ice and incubated for 5 to 30 minutes. The transformation reaction is heat shocked by incubating in a 42° C. water bath for 30 seconds and the reaction is immediately returned to the ice for at least 2 minutes. 250 μL of room temperature SOC media is added to the cells and the tube is incubated sideways in a 37° C. shaking incubator for 1 hour. 100 μl of cells are spread on an LB/Kanamycin (50 ug/ml) plate and incubated overnight at 37° C. Colony PCR is performed in 50 μl reactions on up to 96 colonies using the following reaction conditions: A master PCR mix is prepared for each colony as follows: 35.8 μL sterile water. 5.0 μL 10× ExTaq buffer, 4.0 μL 2.5 mM each dNTPs; 2.5 μL 10 μM primer M13Flong; 2.5 μL 10 μM primer M13Rlong; 0.2 μL ExTaq enzyme. 50 μL of the master mix is dispensed as appropriate into the wells of a PCR plate. Individual colonies are picked with a pipette tip and dropped into the PCR mix. The PCR reaction is run with the following protocol: Denature at 94° C. for 2:00 minutes; 25 cycles: denature at 94° C. for 30 seconds, anneal at 60° C. for 30 seconds, elongate at 72° C. for one minute, the reaction is extended at 72° C. for 5 minutes, and held at 4° C.

i. ExoSAP Cleanup

Excess primers and dNTPs from the PCR products obtained in step h above are removed by treatment with Exonuclease I and Shrimp Alkaline Phosphatase (SAP). Alternatively, samples are cleaned up using Qiagen spin columns (Qiagen, Catalog #28104). Reactions are set up as follows: ExoSAP master mix: per reaction, 3.5 μL dH2O; 0.625 μL 10×SAP buffer; 0.625 μL Exonuclease I; 1.25 μL SAP. 6 μL of the ExoSAP master mix is distributed to the appropriate number of wells of a PCR plate. 19 μL of the PCR reaction of step h above is added to the ExoSAP wells, mixed by pipetting and cycled in a thermocycling conditions for 45 minutes total as follows: 37° C. for 30 minutes, 80° C. for 15 minutes and held at 10° C.

j. Sequencing

ExoSAP cleaned DNA samples are sequenced using ABI automated sequencers. The sequencing is typically sent to one of two commercial vendors, Eton Bioscience (www.etonbio.com) or Genewiz (www.genewiz.com). Alternatively, sequencing is performed on an ABI automated sequencer according to manufacturer's instructions. Primers are presented in Table 3.

k. Data Processing and Analysis

Data is obtained in two different file formats (AB1 and SEQ) and the AB1 file is imported into the SeqMan Pro application from the Lasergene 8 suite of software from DNAstar. Sequences are trimmed of the vector sequence (pCRIITOPO) and are also trimmed of low quality base pairs (stringency high, which corresponds to an average quality score threshold of 16). Sequences are then assembled into contigs based on the following criteria: match size, 12; minimum match % 90; minimum sequence length, 100; maximum added gaps per kb in contig, 70; maximum added gaps per kb in sequence, 70; maximum register shift difference, 70; lastgroup considered, 2; gap penalty, 0.00; gap length penalty, 0.7. Contigs are then exported as a single file (FASTA format). A contig file is uploaded and blasted against the NCBI nucleotide database (NT) using megablast. The top hit by max score is then selected and information on the accession number, the description of the hit and the max score are entered into an Excel spreadsheet, along with information on the length of the contig and the number of sequences that are in the contig.

TABLE 3
List of primers used in PCR amplification of
environmental DNA and vectors.

| Primer | Sequence | | SEQ ID |
|---|---|---|---|
| Name | (5′-3′) | Reference | NO |
| ITS1 + 2 | TCCGTAGGTGAACC | White et al, | 10 |
| forward/ITS1 | TGCGG | | |
| ITS1 + 2 | TCCTCCGCTTATTG | White et al, | 11 |
| reverse/ITS2 | ATATGC | | |
| ITS1 reverse | GCTGCGTTCTTCAT | White et al, | 12 |
| | CGATGC | | |
| ITS2 forward | GCATCGATGAAGAA | White et al, | 13 |
| | CGCAGC | | |
| 18S forward | AACCTGGTTGATCC | Freeman et al, | 14 |
| | TGCCAGT | | |
| 18S reverse | GGGCATCACAGACC | Freeman et al, | 15 |
| | TG | | |
| 28S forward | GTACCCGCTGAACT | Rehner & | 16 |
| | TAAGC | | |
| 28S reverse | TACTACCACCAAGA | Rehner & | 17 |
| | TCT | | |
| 16S forward | TAGATACCCYGGTA | Dewhirst et al, | 18 |
| | GTCC | | |
| 16S reverse | AAGGAGGTGWTCCA | Dewhirst et al, | 19 |
| | RCC | | |
| TOPO cloning | CGACGTTGTAAAAC | Invitrogen | 20 |
| forward | GACGGCCAG | | |
| TOPO cloning | CACAGGAAACAGCT | Invitrogen | 21 |
| reverse | ATGACCATGATTAC | | |

I. Phylogenetic Analysis of Isolated Pests

Pests isolated according to Example 1, steps a to f., are subjected to further sequence analysis according to the methods of Example 1, steps g to k. Multiple sequence alignments are generated using MUSCLE alignment program (Edgar R C (2004). “MUSCLE: multiple sequence alignment with high accuracy and high throughput”. Nucleic Acids Research 32 (5): 1792-97, version 3.8.31) with the processed 18S, 28S and 16S sequences obtained in Example 1, step k.

The 18S sequences are compared to Genbank sequence ID numbers ay635838, ay601707, m62707, dq536481, m62704, dq322625, m62705, m62706, ah009066, ah009067, y17504, af164335, af164337, ay546682, ab016019, af164333, ay635839, af164278, ah009039, ay601711, ah009033, ah009047, ah009046, ah009044, ay546683, ay635844, ah009048, ah009049, ah009043, ay635835, ah009045, dq536475, aj784274, dq536476, dq322623, ay032608, af164253, af051932, ay635826, ay635824, dq536478, af164272, ay601710, af164263, ah009032, ah009051, dq536485, dq536488, dq536492, dq536479, dq322622, ah009034, ay635823, dq536491, af164247, ah009022, af164245, ah009024, m59759, dq536477, dq536490, ay546684, dq536480, ay635830, ah009030, ah009028, ah009027, ay635829, ah009060, ah009053, ah009059, ay635825, dq536482, ay635827, dq536486, ay349035, ay349032, m59758, dq536487, dq536483, ah009063, ah009064, ah009056, dq536473, ah009058, ah009065, ah009055, ah009054, dq536484, ah009057, ay601709, ay349036, ay552524, u23936, ay635842, ah009068, ay635822, af322406, ay635840, dq536472, dq536489, ay601708, dq322624, ay635841, af007533, af13418, ay635820, ay635837, af007540, dq322627, dq322630, ay635832, ay251633 and v01335.

The 28S sequences are compared to Genbank sequence ID numbers dq273803, dq273766, dq273829, dq273822, ay349059, dq273771, dq273777, ay546687, dq273804, dq273814, ay546686, ay349083, ay546688, dq273816, dq273798, dq273819, dq273820, dq273815, ay546693, dq273784, dq273782, dq273824, dq273775, dq273770, dq273835, dq273837, ay439049, dq273823, dq273781, dq273778, dq273776, dq273821, ay546692, dq273826, dq273789, dq273787, ay349097, dq273783, dq273831, dq273785, dq536493, ay349068, dq273836, dq273832, dq273839, ay442957, ay439071, dq273813, dq273838, ay988517, dq273834, ay349063, dq273769, ay439072, ay552525, dq273808, dq273780, dq273767, dq273805, dq273767, dq273818, dq273807, ay546691, ay546689, dq273772, dq273800, dq273773, dq273797, dq273828, dq273792, z19136, j01355, af356652, ay026374, ay026380, dq273802, ay724688, ay026370 and ay026365.

The 5.8S sequences are compared to Genbank sequence ID numbers ay997087, ay997086, ay997042, ay997064, ay349112, ay349128, ay997055, ay997061, ay997060, ay997066, ay997056, ay997074, ay349109, ay997037, ay997044, ay997065, ay997094, ay997095, ay997036, ay997031, ay997048, ay997049, dq536494, ay997077, ay997079, dq536497, dq536500, dq536495, ay997084, ay997082, ay997051, ay997075, ay997093, ay997092, ay997096, ay997033, ay997078, ay997035, ay997083, dq536498, ay349119, dq536499, ay349116, ay997070, dq536501, dq536496, ay997076, ay349115, ay997028, ay997032, ay997034, ay997038, ay997059, ay997072, ay997067, ay997030, ay997039, ay997041, ay997047, ay997071, ay997089, ay997097, ay997054, ay997088, v01361, ay130313, ay227753, ay997029, ay363957, aj627184, and af484687.

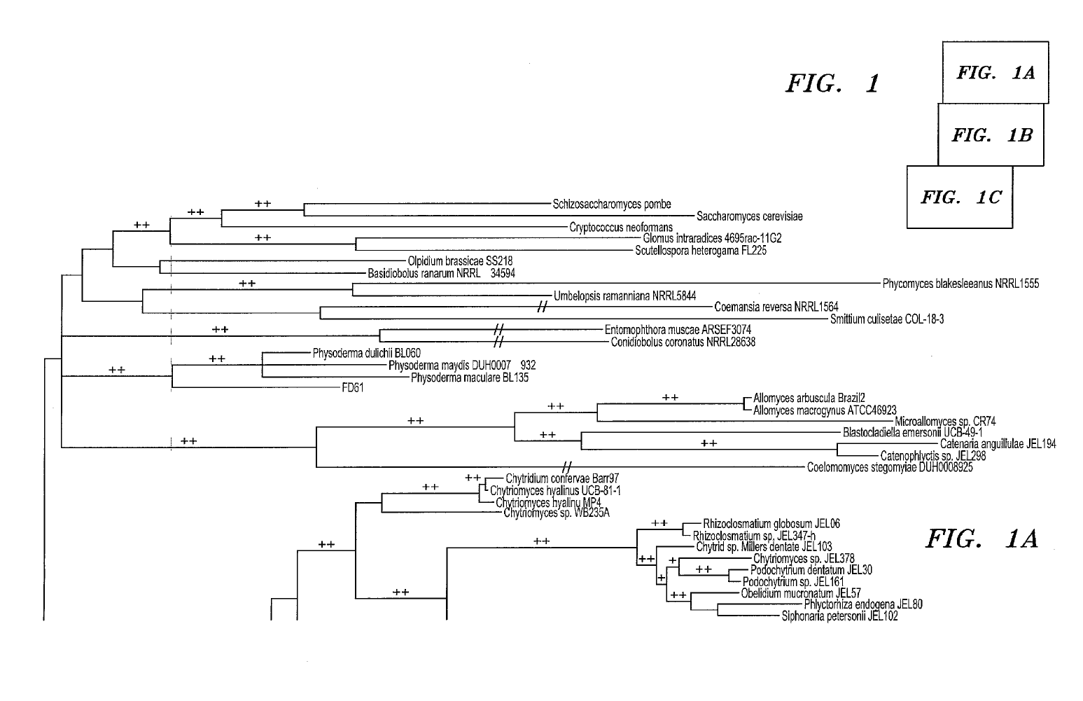

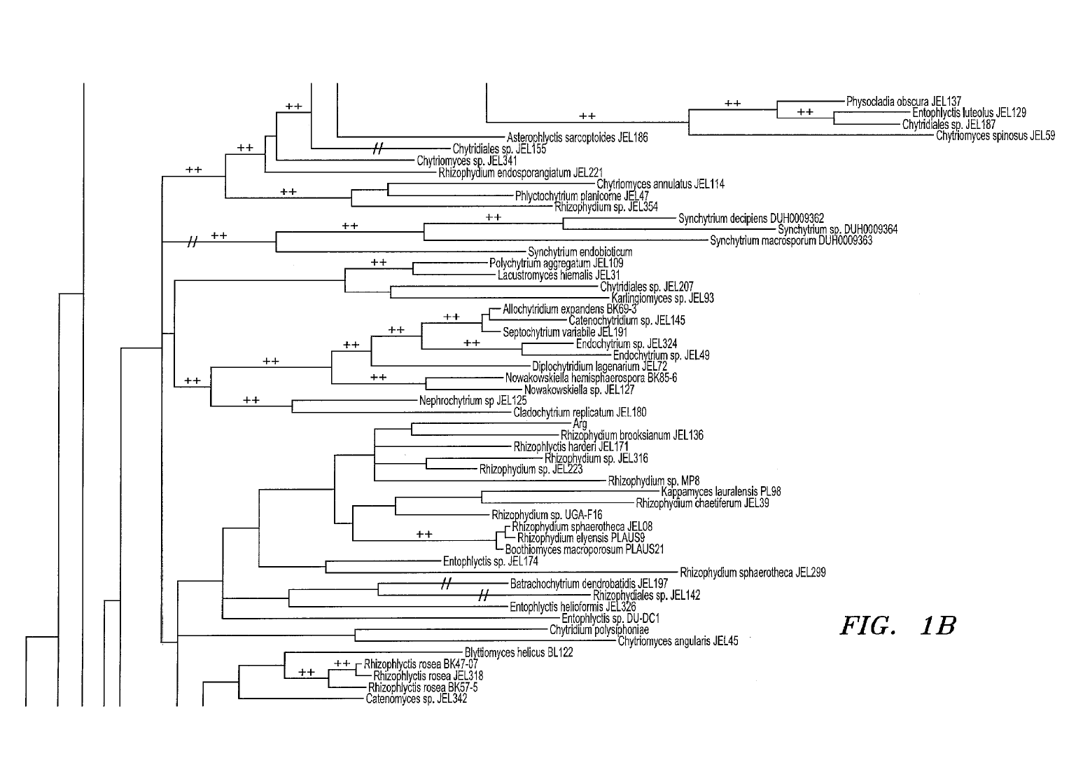

The resulting alignment is manually trimmed and corrected for errors and concatenated for Bayesian analysis using Mr. Bayes version 3.1.2 obtainable from mrbayes.csit.fsu.edu (Ronquist F et al., “MrBayes 3: Bayesian phylogenetic inference under mixed models,” Bioinformatics 19(12): 1572-4 (2003)). The output is converted to phylip format and Maximum liklihood analysis is performed using RAxML (Stamatakis A, et al., “RAxML-III: a fast program for maximum likelihood-based inference of large phylogenetic trees,” Bioinformatics 21(4):456-63 (2005)). The resulting phylogenetic tree is presented in FIG. 1 presenting the results of 4 isolated pests designated FD01, FD61, FD95, Arg.

### Example 2

### Tool Design and Extraction Optimization

Based on the sequence analysis results of Example 1, both specific and universal qPCR primers for the pest sequence are designed and validated for efficiency and specificity on both plasmid DNA and environmental isolated DNA. The qPCR primers are designed to amplify genomic DNA. For each qPCR primer tool, the extraction protocols are validated to ensure that the pest DNA sample isolation is efficient. See, Example 1, steps b to e above. To validate the extraction protocol, a serial dilution of environmental samples is prepared and the efficiency of the extraction methodology is compared.

### Example 3

### Pond Molecular Surveillance

Using the validated molecular tools developed in Example 2, ponds are surveyed on a daily basis for all of the pests identified in Example 1. The pests and sequences used for monitoring are presented in Table 4.

a. DNA Template Preparation:

DNA templates are prepared according to the boiling method of Example 1(b) above. The samples are lysed by heating as follows: for Scendesmus (Desmid) cultures: two cycles: 95° C. for 10 minutes, 25° C. for 5 minutes, hold at 4° C. Nanno cultures: four cycles: 95° C. for 10 minutes, 25° C. for 5 minutes, hold at 4° C. Cyanobacterial cultures cannot be efficiently lysed with just boiling cycles and need to undergo bead beating for 3 minutes to be effectively lysed. See, paragraph [00257], above. The lysed samples are diluted 1:20 with sterile water. Heat sample with the following protocol:

b. qPCR Reactions

10 μl qPCR reactions are prepared in 96-well plates as follows:

| Component | Volume per reaction | |
|---|---|---|
| SsoFast EvaGreen SuperMix (Bio- | 5 | μl |
| Rad, #172-5201) | | |
| 1 μM primer mix (0.5 μM each) | 2.4 | μl |
| DNA template (1:20 diluted) | 2.6 | μl |
| Total volume | 10 | μl |

The reactions in the 96-well plate are centrifuged for 2 minutes at 2,500 rpm. qPCR cycling is performed on a CFX96 cycler (Biorad) using the following conditions.

EVA Green Cycle Conditions with Melt Curve

| Cycle | Repeat(s) | Step | Time | Temp. | Temp. change | Function |
|---|---|---|---|---|---|---|
| 1 | 1 | 1 | 2:00 | 98° C. | | |
| 2 | 40 | 1 | 0:01 | 98° C. | | |
| | | 2 | 0:02 | 57° C. | | Real time |
| 3 | | 1 | 0:10 | 65° C. | 0.5° C. | Melt curve |

Once primers are fully validated, melt curves are omitted to save time using the following qPCR cycling protocol.

| Cycle | Repeat(s) | Step | Time | Temp. | Temp. change | Function |
|---|---|---|---|---|---|---|
| 1 | 1 | 1 | 2:00 | 98° C. | | |
| 2 | 40 | 1 | 0:01 | 98° C. | | |
| | | 2 | 0:02 | 57° C. | | Real time |

Primers are selected to produce a product which is approximately 100 base pairs (bp). Five primer sets for each of the pests are screened and the following primers selected for additional use.

TABLE 4
Primers for qPCR and Pest Identification

| | | | | SEQ |
|---|---|---|---|---|
| | | Internal | | ID |
| Name | ID | id | Sequence | NO. |
| Uncultured | FD0095 | RM-A3.L | CACGCGTACGG | 22 |
| Fungus 167-40 | | | TTGATTAGA | |
| | | RM-A3.R | TGAATGCACTT | 23 |
| | | | TGCACTGCT | |
| Uncultured | FD0001 | RM-B3.L | CCACAAATCCC | 24 |
| Fungus F1210G | | | TGTTACAATCA | |
| | | RM-B3.R | TTACCTGCGTT | 25 |
| | | | ATGCGTGTG | |
| Uncultured | FD0061 | RM-C2.L | GATCAAAACCG | 26 |
| Fungus IVN1-23 | | | CTCACCAAT | |
| | | RM-C2.R | TGAATTGCAGA | 27 |
| | | | ACTCCGTGA | |
| Uncultured | FD100 | YX-F | ATGTCATTGGG | 28 |
| Chytrid | | | ATTGCCTCT | |
| | | YX-R | CGGGTCCTCCT | 29 |
| | | | ACCTGATTT | |
| Methyltransferase | Uni- | YX-F | GGGCGTACCAT | 30 |
| gene | versal | | AATCTGCAT | |
| | | YX-R | ATGACACCGTC | 31 |
| | | | AGGAAAACG | |
| ITS gene | Uni- | YX-F | CGGACCAAGGA | 32 |
| | versal | | GTCTAACA | |
| | | YX-R | TTGCACGTCAG | 33 |
| | | | AATCGCTAC | |
| Scenedesmus | SE0004 | YX-D1.L | TACCCTCACCC | 34 |
| sp. BR2 | | | CTCTCTCCT | |
| | | YX-D1.R | TAAGCTTCAGC | 35 |
| | | | CAACCCAAT | |
| Nannochloropsis | SE0087 | RM- | CTGGGATATCG | 36 |
| salina | | SE0087 3L | TCGCTCCTA | |
| | | RM- | ATGGGTATGCG | 37 |
| | | SE0087 3R | TCCGTTAGA | |

### Example 4

### Pond Non-Molecular Surveillance

Ponds are also surveyed daily using non-molecular tools to provide an indication of a health or level of infection in a particular pond. One or more of the following attributes are assessed.

Detection of Chytrid Infection of Growing Microalgae Cultures Using Calcofluor White M2R.

A 1.5 ml. sample of culture is obtained and incubated with a 1% solution of Calcofluor White M2R (Sigma, Cat. #18909) for 10 minutes in the dark. Pellets are obtained by centrifugation at 20,000 g for 15 minutes and resuspended in 250 μl of water. A 2-fold dilution series is prepared (1:1 to 1:128) in a 96 well plate and fluorescence measured on a SpectraMax fluorescent plate reader. Fluorescence is measured simultaneously at the wavelengths presented in Table 5.

TABLE 5
Excitation and emission spectra for chytrid detection

| Target | Excitation (run) | Cutoff (nm) | Emission (nm) |
|---|---|---|---|
| Calcofluor White | 360 | 435 | 444 |
| Chlorophyll | 430 | 665 | 685 |

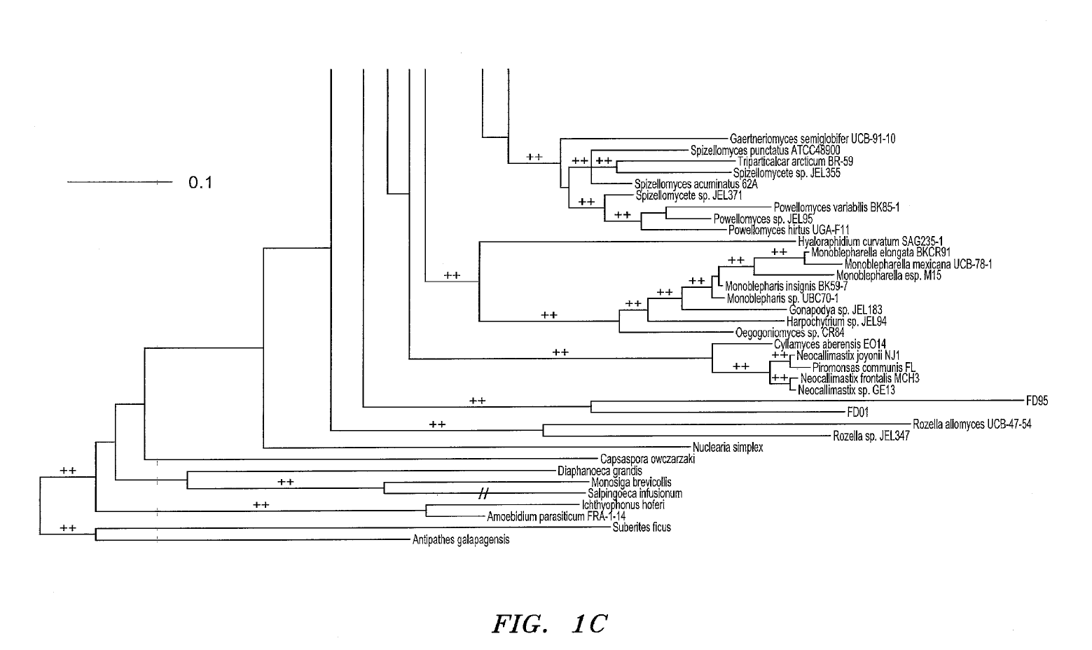

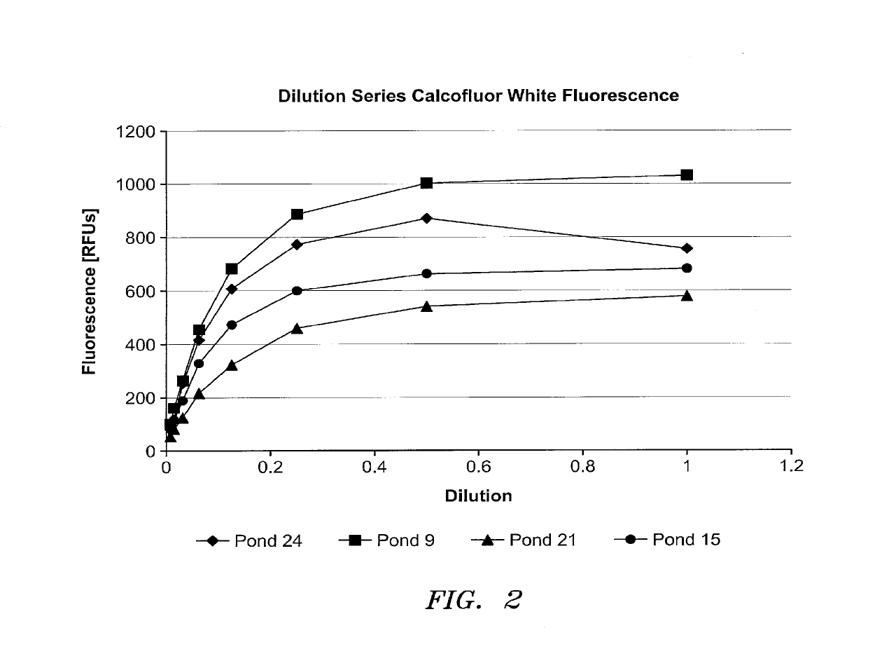

The results of a Calcofluor White binding assay are presented in FIG. 2. Pond 9 has the highest level of fluorescence corresponding to a higher level of chytrid infection while Pond 21 has a lower level of chytrid infection. The level of infection of Pond 24 and Pond 15 are intermediate to the infection levels of Pond 9 and Pond 21.

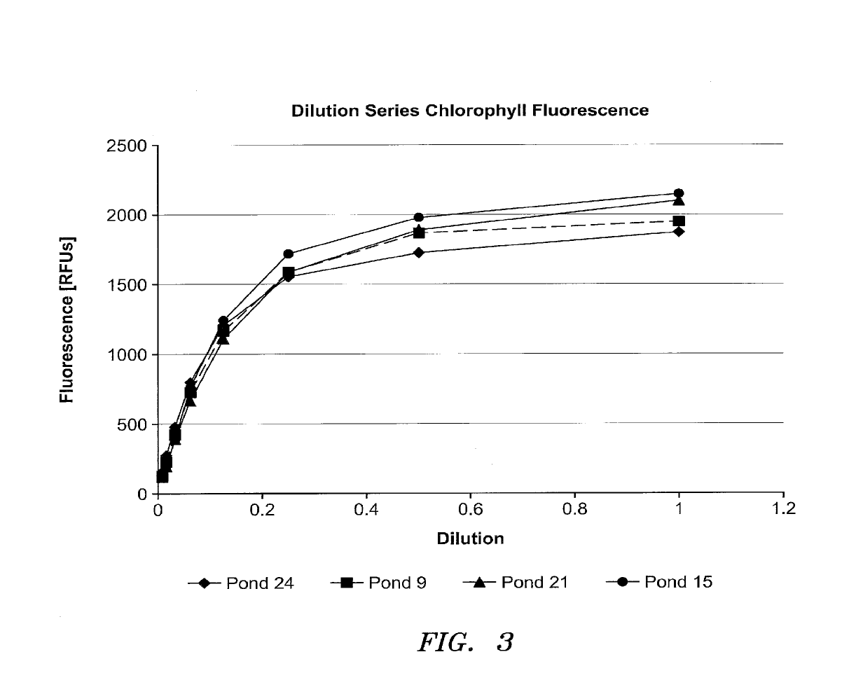

In contrast to the differential Calcofluor White fluorescence presented for Ponds 9, 15, 21, and 24 in FIG. 2, measurement of chlorophyll fluorescence does not present significant differences as shown in FIG. 3.

Calcofluor White treated samples are further examined microscopically under a DAPI filter for the presence of chytrids. An example of a Calcofluor white binding assay is shown in FIG. 4. As shown in the left image of panel A, desmid chlorophyll fluorescence is detected while the right image does not have a fluorescence emission at 444 nm. In panels B-D, the presence of chydtrids identifiable by SEQ ID NOs: 1 to 3 is detected as demonstrated by the fluorescence in the right image of each panel.

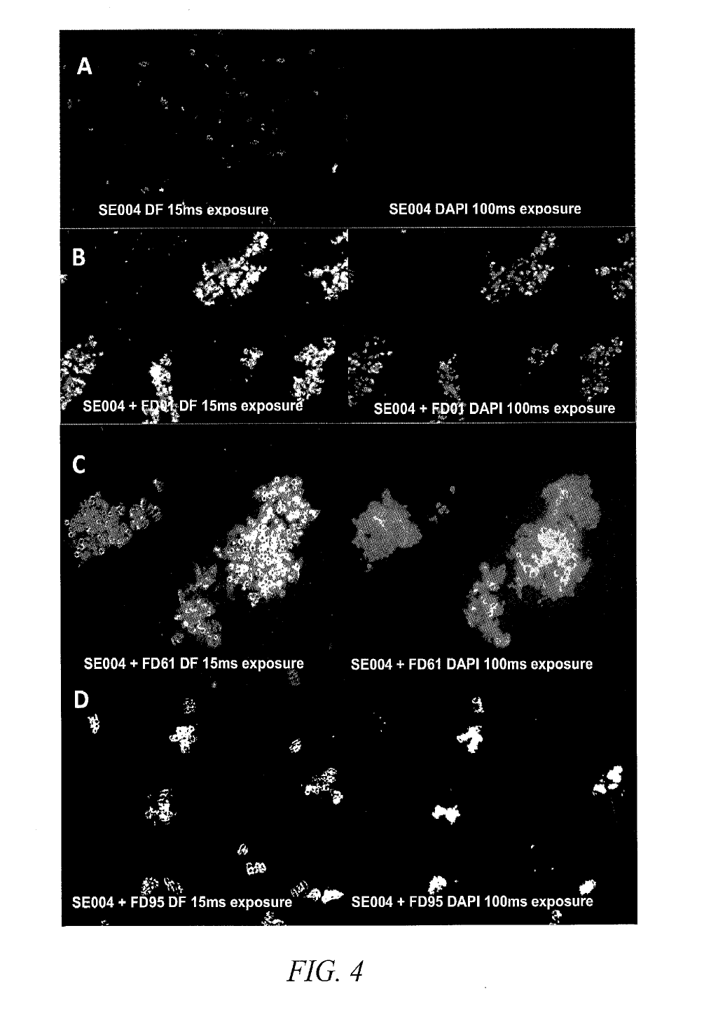

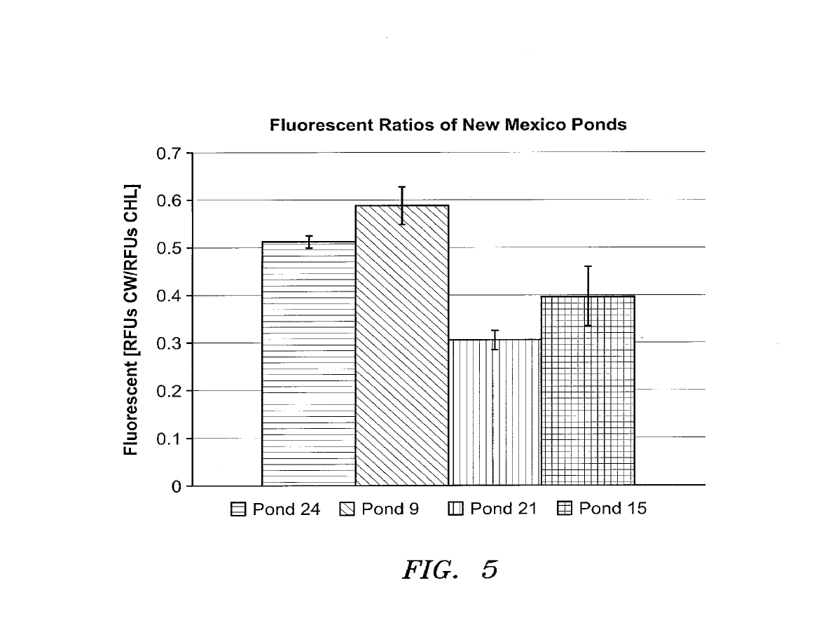

The ratio of Calcofluor white to chlorophyll fluorescence provides an indication of the health or level of infection in a particular pond. FIG. 5 presents the fluorescence ratio of four ponds. As can be seen, Pond 9 has a higher ratio corresponding to a higher level of chytrid infection while Pond 21 has a lower ratio and a corresponding lower level of chytrid infection. The fluorescence ratio of Ponds 15 and 24 are intermediate to Ponds 9 and 21.

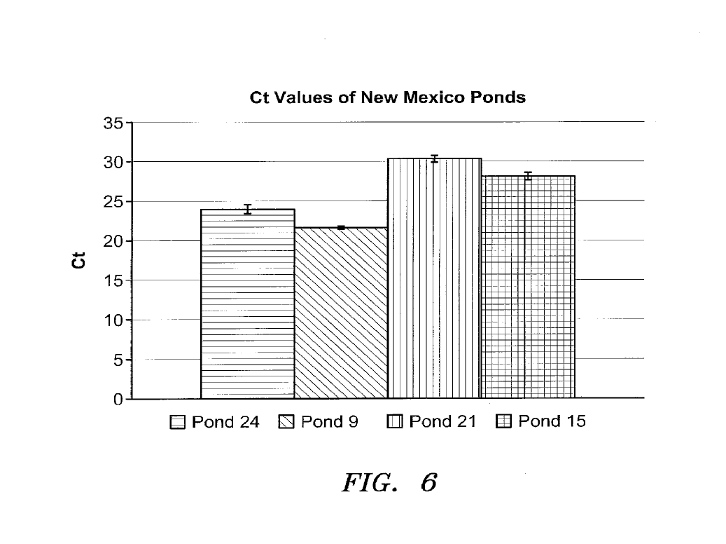

The correlation between the Calcofluor white to chlorophyll fluorescence ratio and chytrid infection level is confirmed by PCR. In FIG. 6, higher levels of chytrid infection are evident as a lower Ct value for Pond 24 and Pond 9. Similarly, decreased levels of infection are observed a higher Ct value for Pond 21 and Pond 15. The relative levels of chytrid infection determined by Calcofluor white to chlorophyll fluorescence and by PCR are the same: Pond 9>Pond 24>Pond 15>Pond 21.

The health of a microalgae culture is further monitored using a flocculation assay. 5 ml of culture is obtained and placed in a 17×100 mm culture tube. A sample is obtained from a predetermined depth at time zero (T₀) and at 40 minutes (T₄₀) and the OD750 and chlorophyll fluorescence determined. The settling rate is determined as the T₄₀/T₀ ratio. When the ratio goes below 0.35, an indepth biological review of the pond is performed including, for example, qPCR, dye binding, fluorescence and other methods as provided above.

### Example 5

### Threshold Determination and Crop Protective Action

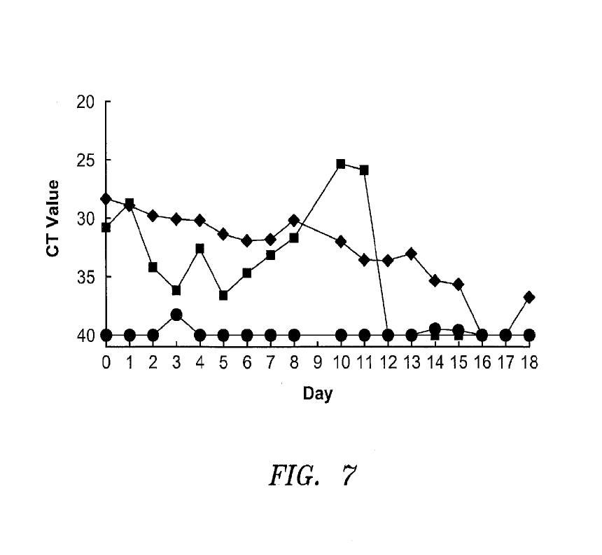

Based on daily monitoring using the methods of Example 4, ponds in need of protective action are identified and treated. For each pest identified in Example 1 and validated in Example 2, a threshold is identified which is pest specific. For chytrid pests identifiable using SEQ ID NOs: 1 to 3, a consistent decrease in Cₜ of less than 30 indicates a need for crop protective action. The results of monitoring are presented in FIG. 7.

Crop protective action is indicated by the threshold Cₜ for each continuously monitored pond. Upon indication, a first fungicide is added at a predetermined concentration (Headline® 1 ppm, Omega® 0.5 ppm, Thiram® 1 ppm) by a licensed applicator and monitoring is continued. If the Cₜ threshold is reached again, a different fungicide (second fungicide) is added at a predetermined concentration (Headline® 1 ppm, Omega® 0.5 ppm, Thiram® 1 ppm) by a licensed pesticide applicator and monitoring is continued. To avoid the development of resistant pests, fungicides are rotated based on the mode of action. For example, three fungicides are rotated in outdoor ponds: Headline® (Pyraclostrobin) and Omega® (Fluazinam) and Thiram®-42WP (Thiram®). Headline® is a strobilurin and acts to inhibit the respiratory chain. Omega is a pyridine fungicide which acts to inhibit cellular energy production. Thiram® is a sulfide which acts on multiple sites in the respiratory pathway. Effectiveness of treatment is monitored using both molecular and non molecular means post treatment (FIG. 7).

One of the chytrids' population begins to increase around the 6^{th} day of this graph and increases consistently. Once it crosses a Cₜ threshold value of 30 and shows a consistent increase of more than 3 cycle thresholds, the pond is treated with a 2 ppm dose of Headline®. The pond is continuously monitored and chytrid activity ceases as a result of the treatment.

### Example 6

### Identification of Effective Fungicides

Algae are screened for sensitivity to chemicals by preparing 180 ml of a log phase culture. The log phase culture is transferred into a 96 well microtiter plate at an absorbance at 750 nm (OD750 or A750) of ˜0.2. Twenty microliters of media is provided into the top row as a negative control, the middle six rows receive a 20 microliter dilution, which is a 10 fold dilution of the chemical at each transfer, of the chemical across an appropriate concentration gradient, and the bottom row receives 20 microliter of the solvent used to solubilize the pesticide alone as a control. The total volume per well is 200 μl. Each chemical is tested in triplicate. The growth of the algae is tracked daily by measuring the A750. After 8 days, the growth rate of the algae is measured by fitting the growth curve to a log model and deriving the maximum growth rate (r). The impact of the chemical is calculated by comparing the r of the algae at various dilutions of the pesticide to the control.

TABLE 6
Effectiveness of Fungicides on Pest Control and Microalgae Growth

| | Desmid | Scenedesmus | Scenedesmus | Scenedesmus | Scenedesmus | |
|---|---|---|---|---|---|---|
| | species | dimorphus | species | dimorphus | dimorphus | |
| | toxicity | toxicity | toxicity | efficacy | Efficacy | |
| Description | (ppm) | (ppm) | (ppm) | (ppm) | confirmation | Rating |
| acibenzolar | | | | 17.5% @ 0.8 ppm | | |
| azoxystrobin | | | | — | | 0 |
| benodanil | n/d | 0.3125-1.25 | 0.3125-1.25 | 38.7% @ 5 ppm | (−) | 0 |
| binapacryl | n/d | 0.125-0.5 | 0.125-0.5 | — | | |
| boscalid | | | | — | | 0 |
| bronopol | | | | 14.3% @ 5 ppm | | 0 |
| captan | 31.25-125 | 1.953-7.813 | 31.25-125 | — | | 0 |
| carbendazirn | | | | 26.7% @ 5 ppm | (−) | 0 |
| carboxine | 0.977-3.906 | 0-0.244 | 0.244-0.977 | 24.6% @ 5 ppm | (−) | 0 |
| chlorothalonil | | | | 84.5% @ 2 ppm | 75.7% @ 2 ppm | 3 |
| cyazofamid | 0.125-0.500 | 0.03125-0.125 | 0.03125-0.125 | (−) | | 0 |
| cymoxanil | 15.625-62.5 | 15.63-62.5 | 15.63-62.5 | (−) | | 0 |
| cyprodinil | | | | 8.1% @ 2 ppm | | 0 |
| dibromocyanoacetamide | | | | 35.4% @ 5 ppm | (−) | 0 |
| dimoxystrobin | 0.004-0.0156 | 0.0156-0.0625 | 0.0156-0.0625 | (−) | | 0 |
| dinocap | n/d | 0.0039-0.0156 | 0.0156-0.0625 | (−) | | 0 |
| diquat dibromide | | | | | | |
| dithianon | n/d | 0.625-2.5 | n/d | 37% @ 5 ppm | 52.2% @ 5 ppm | 2 |
| dodemorph | N/A | 0.195-0.781 | 0.195-0.781 | (−) | | 0 |
| dodine | | | | 93.9% @ 2 ppm | 100% @ 2 ppm | 3 |
| endothal monohydrate | | | | | | |
| fenarimol | N/A | 0.0489-0.195 | 0.0489-0.195 | 31% @ 2 ppm | (−) | 0 |
| fenhexamid | 15.63-62.5 | n/d | n/d | (−) | | 0 |
| fenpropidin | 0.031-0.125 | 0.00195-0.0078 | 0.488-1.953 | 18.5% @ 0.8 | (−) | 0 |
| | | | (ppb) | ppm | | |
| fluazinam | | | | 67% @ 0.8 ppm | | 2 |
| fluoxastrobin | | | | | | |
| fosetyl-aluminum (100 mg) | | | | (−) | | 0 |
| kresoxim-methyl | | | | (−) | | 0 |
| mancozeb | | | | (−) | | 0 |
| metalaxyl | | | | (−) | | 0 |
| methyl isothiazolin | | | | | | |
| nystatin | | | | | | |
| oryzalin | | | | | | |
| pencycuron | 3.125-12.5 | 3.125-12.5 | 0.781-3.125 | (−) | | 0 |
| propamocarb | 3.906-15.625 | 15.63-62.5 | 3.906-15.63 | (−) | (−) | 0 |
| propiconazole | | | | 30.6% @ .32 ppm | 18.6% @ 0.8 ppm | 0 |
| prothioconazole | | | | (−) | | 0 |
| pyraclostrobin | | | | 66% @ 2 ppm | | 2 |
| pyrifenox | 0.004-0.0156 | 0.039-0.156 | 0.039-0.156 | (+/−) | (−) | 0 |
| sonar | | | | | | |
| spiroxamine | 0.250-1.0 | 0.0625-0.250 | 0.250-1.0 | (−) | | 0 |
| tebuconazole | | | | (−) | | 0 |
| temefos | | | | | | |
| terbuthylazine | | | | | | |
| thiophanate-methyl | | | | 9.6% @ 5 ppm | | 0 |
| Thiram ® | | | | (+/−) | 23.1% @ 5 ppm | 1 |
| tolylfluanid | 6.25-25.0 | 1.56-6.25 | 6.25-25.0 | 5 ppm Delayed Crash | | 0 |
| triadimenol A | | | | 33.0% @ 0.8 ppm | (−) | 0 |
| triclopyr | | | | | | |
| trifloxystrobin | | | | (−) | | 0 |
| triflumizole | 1.563-6.25 | 1.563-6.25 | 1.563-6.25 | (−) | | 0 |
| trifluralin | | | | | | |
| triforin | | | | (−) | | 0 |
| zoxamide | 0.0156-0.0625 | 0.0625-0.250 | 0.0156-0.0625 | (−) | | 0 |

n/d = not detected, N/A = not applicable, n/t = not tested, (+/−) = indeterminate, (−) ineffective

Efficacy is evaluated at the effective concentration of fungicide as indicated and the percent value is the percent of growth of a treated culture compared to an uninfected control growth rate. Failure to treat an infected culture results in the collapse and loss of the microalgae culture. For some fungicides, a positive efficacy in a first trial is not confirmed in a second trial (See, e.g., benodanil, carbendazim, carboxine, dibromocyanoacetamide, fenarimol, fenpropidin, and triadimenol A at columns 5 and 6). Test fungicides are graded on a rating scale of 0 to 3 where a score of 0 represents an efficacy of between 0 and 25%, a score of 1 represents an efficacy of between 26 and 50%, a score of 2 represents an efficacy of between 51 and 75% and a score of 3 represents an efficacy of between 76 and 100%.

### Example 7

### Effect of Fungicides on Microalgae Growth

a. Effect of Fluazinam

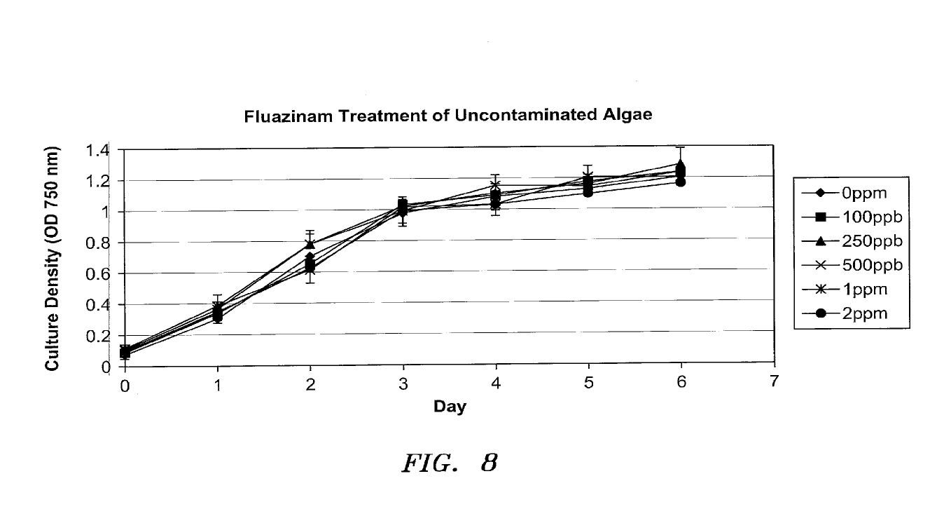

A desmid strain (UTEX 1237) is inoculated into 1 ml of media at an initial A750 of 0.15. The media comprises 1.929 g/L sodium bicarbonate, 0.1 g/L urea, 2.3730 g/L sodium sulfate, 0.52 g/L sodium chloride, 0.298 g/L potassium chloride, 0.365 g/L magnesium sulfate, 0.084 g/L sodium fluoride, 0.035 mL/L 75% phosphoric acid, 0.018 g/L Librel® Fe-Lo, 0.3 mL/L 20× iron stock solution (20× iron stock solution: 1 g/L sodium ethylenediaminetetraacetic acid and 3.88 g/L iron chloride) and 0.06 mL/L 100× trace metal stock solution (100× trace metal stock solution: 1 g/L sodium ethylenediaminetetraacetic acid, 7.2 g/L manganese chloride, 2.09 g/L zinc chloride, 1.26 g/L sodium molybdate, and 0.4 g/L cobalt chloride. Cultures are maintained at 32° C. under constant lighting (˜200 microeinsteins) with shaking and a CO₂ level of approximately 20 000 ppm. These cultures are monitored daily for growth by measuring the optical density of the culture at 750 nm. If pests are detected, their genomic DNA is quantitated at the beginning and end of the experiment using the methods presented above. Uncontaminated laboratory cultures of microalgae are observed in the presence of increasing amounts of the fungicide fluazinam. As shown in FIG. 8, fluazinam concentrations up to 2 ppm do not significantly affect the growth of the uncontaminated microalgae culture.

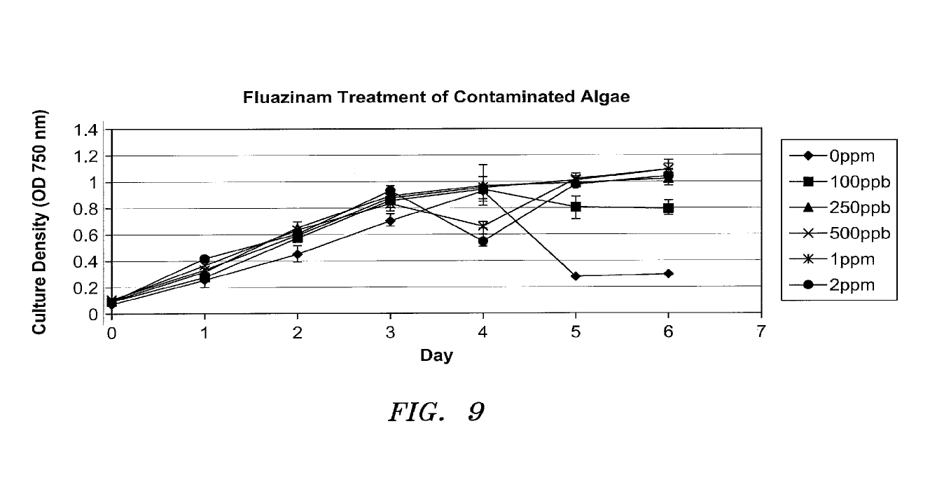

Microalgae cultures are prepared as described above and further inoculated with chytrids known to infect the strain, grown, and monitored as described above. As shown in FIG. 9, the optical density of microalgae in a contaminated culture grown in the absence of fluazinam collapses at day 4 and the optical density does not recover. In contrast, contaminated cultures grown in the presence of 250 ppb or higher concentrations of fluazinam are not affected by the presence of added chytrid. Fluazinam at a concentration of 100 ppb results in a stabilization of microalgae density at 0.8 ODU which is about 4 times the density of microalgae grown in the absence of fluazinam.

b. Effect of Headline®

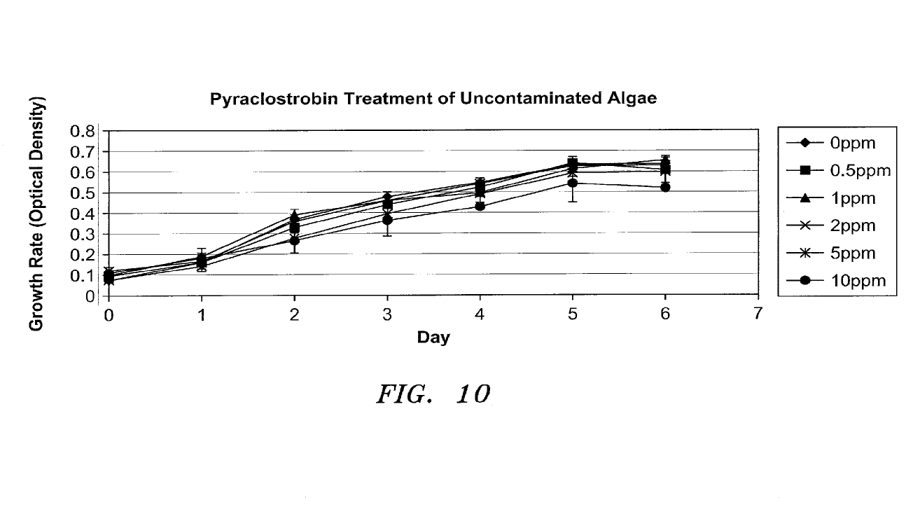

A desmid strain (UTEX 1237) is inoculated into 1.0 ml of media IABR6 at an initial OD (A750) of 0.15. Cultures are maintained at 32° C. under constant lighting (˜200 microeinsteins) with shaking and a CO₂ level of approximately 20,000 ppm. These cultures are monitored daily for growth by measuring the optical density of the culture at 750 nm. If pests are detected, their genomic DNA is quantitated at the beginning and end of the experiment using the methods presented above. Uncontaminated laboratory cultures of microalgae are observed in the presence of increasing amounts of the fungicide Headline®. As shown in FIG. 10, Headline® concentrations up to 2 ppm do not significantly affect the growth of the uncontaminated microalgae culture.

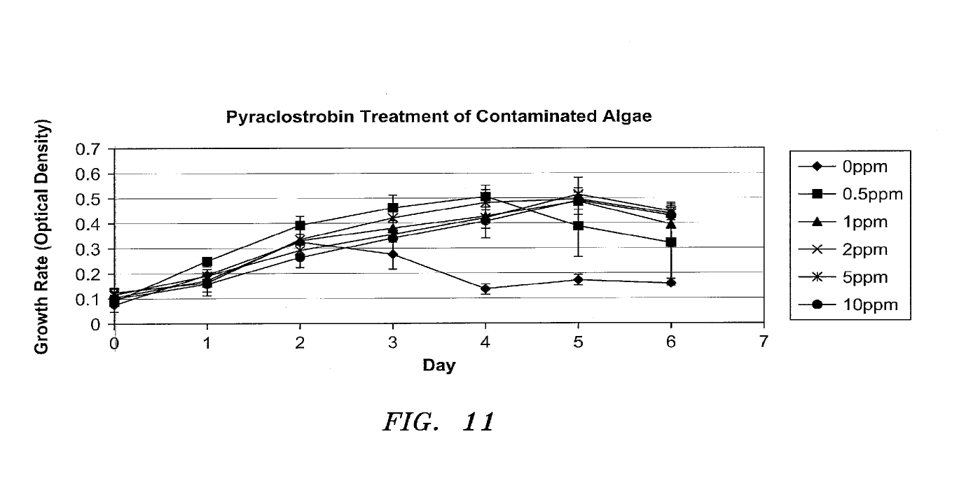

Microalgae cultures are prepared as described above and further inoculated with chytrids known to infect the strain, grown and monitored as described above. As shown in FIG. 11, the optical density of microalgae in a contaminated culture grown in the absence of Headline® collapses beginning at day 2 and the optical density does not recover. In contrast, contaminated cultures grown in the presence of 1 ppm or higher concentrations of Headline® are not affected by the presence of added chytrid contaminant. Headline® at a concentration of 0.5 ppm results in a stabilization of microalgae density at about 0.3 ODU which is about 2 times the density of microalgae grown in the absence of Headline®.

c. Effect of Thiram®

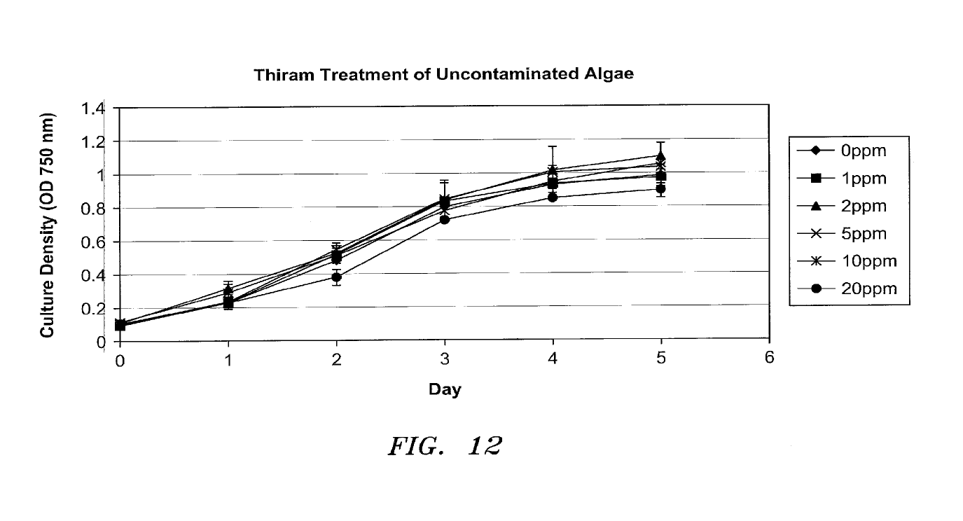

A desmid strain (UTEX 1237) is inoculated into 1 ml of media at an initial OD (750 nm) of 0.15. Cultures are maintained at 32° C. under constant lighting (˜200 microcinsteins) with shaking and a CO₂ level of approximately 20 000 ppm. These cultures are monitored daily for growth by measuring the optical density of the culture at 750 nm. If pests are in these cultures their genomic DNA is quantitated at the beginning and end of the experiment. Uncontaminated laboratory cultures of microalgae are observed in the presence of increasing amounts of the fungicide Thiram®. As shown in FIG. 12, Thiram® concentrations up to 2 ppm do not significantly affect the growth of the uncontaminated microalgae culture.

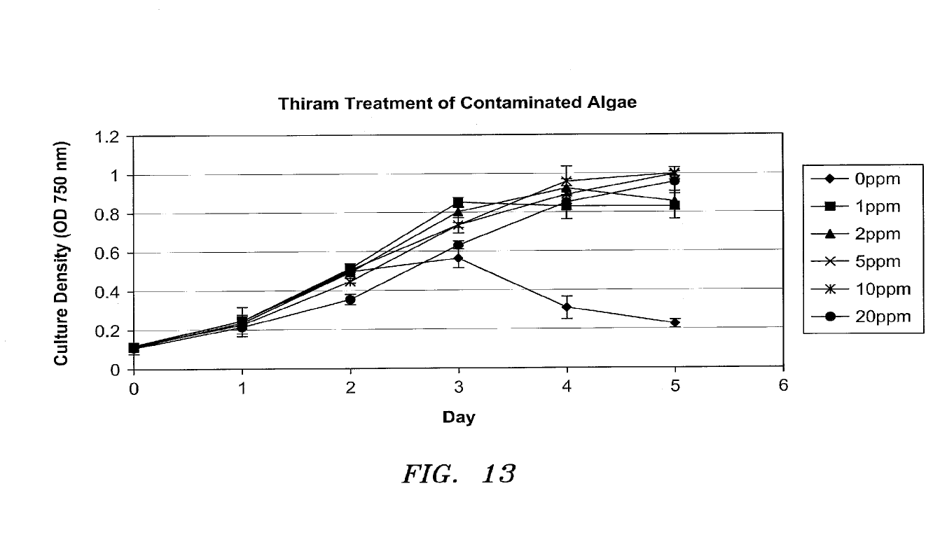

Microalgae cultures are prepared as described above and further inoculated with chytrids known to infect the strain, grown and monitored as described above. As shown in FIG. 13, the optical density of microalgae in a contaminated culture grown in the absence of Thiram® collapses beginning at day 2 and the optical density does not recover. In contrast, contaminated cultures grown in the presence of 2 ppm or higher concentrations of Thiram® are not affected by the presence of added chytrid contaminant. Thiram® at a concentration of 1.0 ppm results in a stabilization of microalgae density at about 0.8 ODU which is about 4 times the density of microalgae grown in the absence of Thiram®.

### Example 7

### Monitoring and Treatment of Ponds

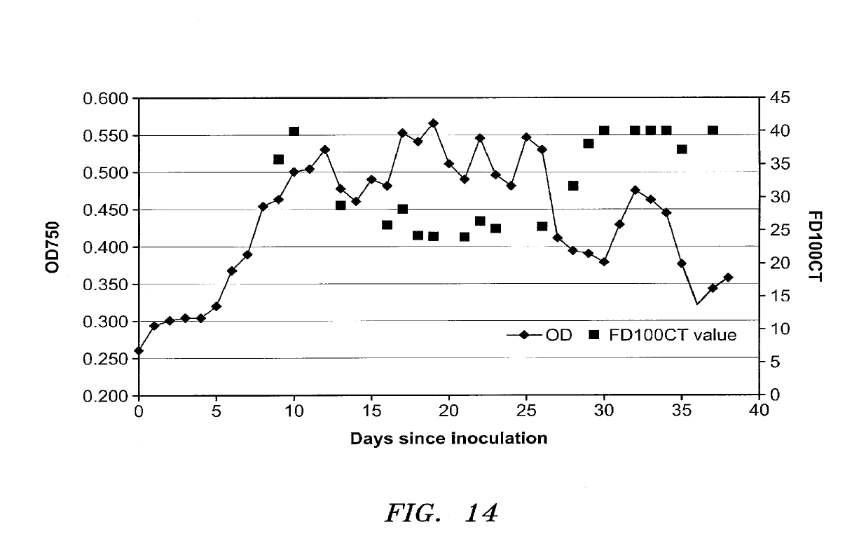

A 200,000 liter outdoor pond located in Las Cruces, N. Mex. is inoculated with a desmid strain at an initial OD (750 nm) of ˜0.15 (pond P08). Growth of the microalgae is monitored daily by measuring the ash free dry weight of the culture. PCR monitoring is performed daily to detect the presence of pests. The results of culture growth monitoring and monitoring of a pest using qPCR primers B7 and B8 are presented in FIG. 14. Additional ponds, monitoring and treatment according to these methods are provided in Table 7 below.

A pond is further monitored using fluorescent dye binding assays as described in Example 4 above. Samples are also examined for fungal contamination using Solapehnyl flavine fluorescent dye staining using the same protocol. Solaphenyl flavine staining is measured using an excitation wavelength of 365 nm and emission is detected at 515 nm. Microscopic examination is performed using a FITC filter.

Samples are further examined for the growth of microalgae by detecting chlorophyll fluorescence. Chlorophyll fluorescence in a desmid culture is measured using an excitation wavelength of 430 nm and an emission wavelength of 685 nm. The results of microalgae growth is used to prepare a semi-log plot of chlorophyll fluorescence versus time to identify growth phases and prepare harvest schedules.

The health of a microalgae pond is further evaluated using a flocculation assay. Samples are obtained from the growing pond and placed in 17×100 mm culture tubes. 200 μl samples are taken from the same depth of the tube at T₀ and at 40 minutes. The settling rate is determined as the ratio of OD750 or the chlorophyll fluorscence at T₄₀/T₀. Ponds are monitored using a FlowCAM. FlowCAM analysis integrates flow cytometry and microscopy allowing for high-throughput analysis of particles in a moving field. Diluted (1:10) culture samples are run through the FlowCAM with a 20× objective (green algae) or a 4× objective (blue-green algae). The FlowCAM and its integrated software automatically images, counts, and analyzes a predetermined amount of particles (typically 3,000) in a continuous flow. Phenotypic attributes (e.g. green vs. transparent cells, large cells vs. small cells, etc.) are recorded.

Initially, the Cₜ value for the pest is above the threshold of Cₜ=30 until day 15 (See, FIG. 14). The measured Cₜ observed for pest FD100 monitoring stays below Cₜ=30 and indicates a need for crop protective action. On day 18 post-inoculation, fluazinam is added at a concentration of 0.5 ppm. Daily monitoring is continued and the Cₜ observed for pest FD100 increases above the Cₜ=30 threshold and remains above the threshold through day 38.

Successful culture depends on the timely identification of a pest. On day 30, in response to a decrease in optical density of the culture, 1 ppm of pyraclostrobin is added. Despite the addition of a second fungicide, the culture collapses.

Additional examples of microalgae growing ponds monitored, treated and harvested are presented in Table 7.

TABLE 7
Monitoring, Treatment and Harvest of
Ponds Growing Scenedesmus Species

| Pond #- | Volume | Length | | |
|---|---|---|---|---|
| Run # | (L) | (days) | Treatments | Harvests |
| P1-1 | 6145 | 42 | 0 | 10 |
| P1-2 | 6145 | 40 | 7 | 1 |
| P1-3 | 6145 | 14 | 4 | 1 |
| P1-4 | 6145 | 7 | 1 | 1 |
| P8-1 | 101822 | 74 | 0 | 6 |
| P16-1 | 346400 | 85 | 6 | 15 |
| P22-1 | 26530 | 108 | 7 | 21 |
| P28-1 | 26530 | 95 | 0 | 10 |

### Example 8

### Growth of Microalgae Outdoor Ponds with or without Fluazinam Treatment

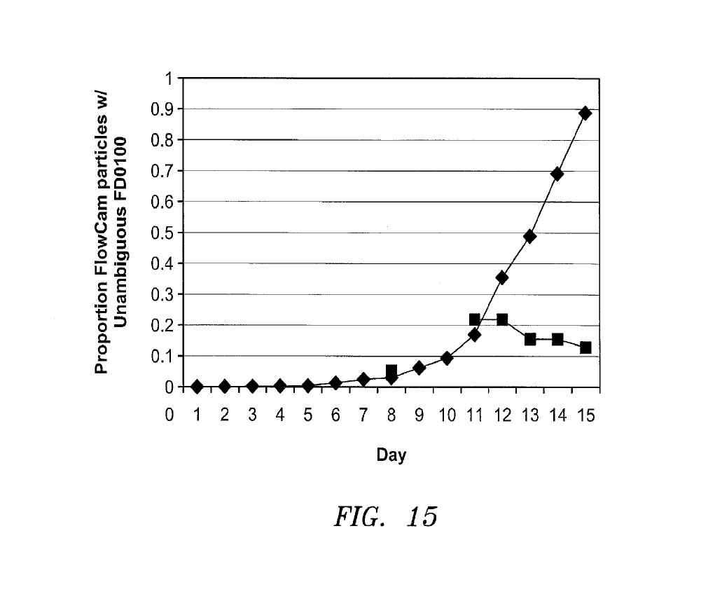

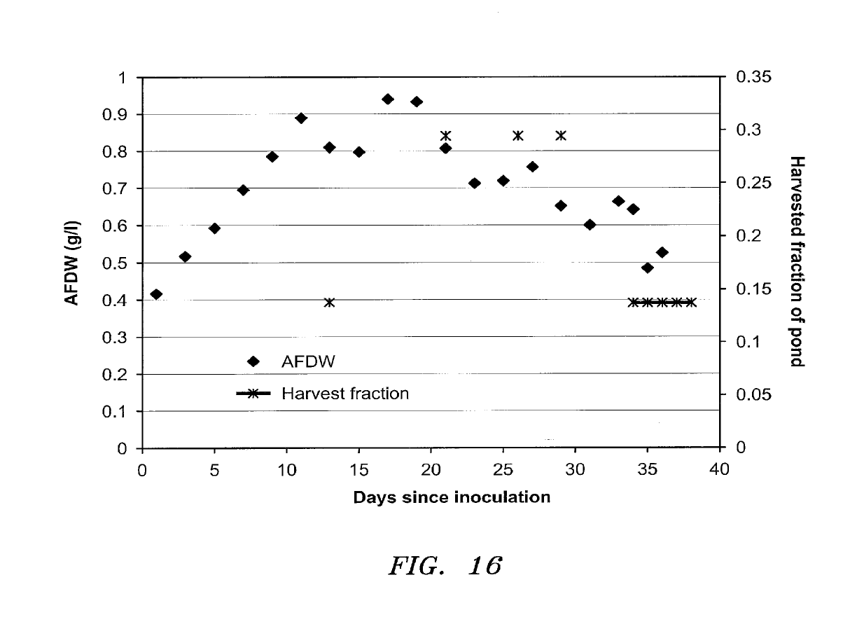

Monitoring for chytrid pest and microalgae growth is performed as described in the examples above. In Pond 16 (P16), a signal for the presence of a chytrid pest is detected beginning on Day 8 using the universal primer pair to the methyltransferase gene (SEQ ID NOs: 30 and 31) and to the ITS gene (SEQ ID NOs: 32 and 33). At this point, 400 liters of P16 are inoculated into Pond A6 (PA6). Continuous detection of the presence of chytrid in pond P16 and A6, indicates a need for crop protection and 0.5 ppm of fluazinam is added on Day 11 to P16 but not PA6. Monitoring of the growth of microalgae is continued in both Pond 16 and Pond A6 using total organic carbon (TOC), OD(750), fluorescence, and a FlowCAM®. Logarithmic growth continues in the fluazinam treated pond P16 while the growth of microalgae in Pond A6 collapses. FIG. 15 shows how the fraction of infected cells in the samples from the ponds increases in PA6 and stabilizes or decreases in P16 after treatment.

### Example 9

### Harvesting of Microalgae

A 500,000 liter outdoor pond located in Las Cruces, N. Mex. is inoculated with a desmid strain at an initial OD (750 nm) of ˜0.15 (Pond 17). Growth and health of the pond are monitored using the methods described in the examples above. Growth and yield problems resulting from an active infection by chytrid FD100 became a problem when the pond reached 1 g/l AFDW (Ash Free Dry Weight). In addition to treatment with a fungicide, harvesting may be initiated to maintain the culture in logarithmic phase at a target OD of 0.3 to 0.4 g/l of biomass (e.g., below an AFDW of 1 g/l) to decrease the virulence of the chytrid pest. Continuous harvesting to maintain logarithmic phase for the algae provides an environment that is less susceptible to FD100 infection. Harvesting is continued to maintain the microalgae culture at an optimal logarithmic growth phase. Optimal harvesting strategies are determined for each species and strain of microalgae.

### Example 10

### Desmid Growth in a ˜500,000 Liter Liquid System

A liquid system having an approximate volume of 500,000 liters (Pond 16, P16) and a depth of about 250 mm is prepared with the media described in Example 7.

The liquid system is inoculated with a desmid on Day 0 (T₀) and growth is monitored for the following parameters: pH, temperature, depth (to account for evaporation), OD750, PAM (Pulse Amplitude Modulated), conductivity, alkalinity, nitrates, phosphates, AFDW (Ash Free Dry Weight), TOC (Total Organic Carbon), and chytrids by qPCR. A FlowCAM® and microscope are used to evaluate the health of the culture. As HNO₃, H₃PO₄, urea, iron and trace metals are depleted, they are added to restore the nutrients to initial levels.

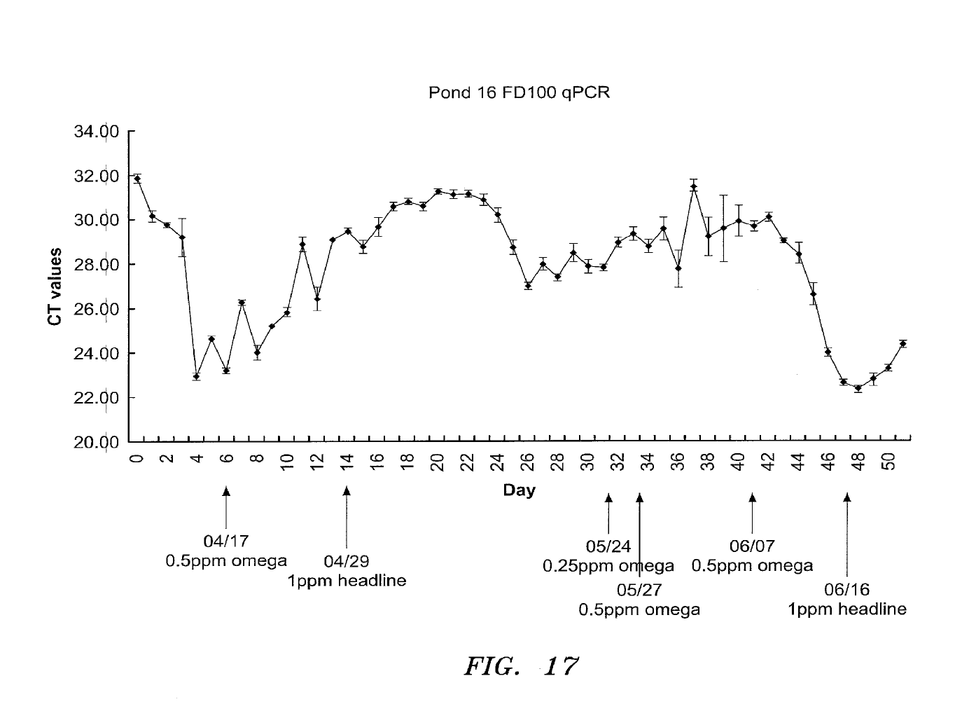

When the OD750 reached approximately 0.6, the desmid is harvested on days 14, 20, 22, 27, 30, 34, 35, 36, 38, 42, 51, 72, 78 and 84 by Disolved Air Flotation device (DAF). Quantitative PCR monitoring is performed daily for the chytrid FD100 using the primers described above. As indicated by the qPCR, P16 is dosed with either Omega® or Headline® on days 19, 31, 57, 59, 70 and 79 as provided in FIG. 17.

Ponds dosed with the indicated fungicide are provided with a volume of fungicide calculated based on the selected concentration dose and the volume of the pond being treated. The calculated volume of fungicide is diluted into 1 L of media and slowly added behind the paddle wheel of the pond. The concentration of fungicide is monitored by collecting 50 ml samples beginning at T₀ and at least every 24 hours. Samples are filtered with a 0.22 uM syringe filter into 50 ml screw top tubes. Samples are immediately stored at −20° C. and thawed for analysis of fungicide levels by HPLC.

### Example 11

### Monitoring and Treatment of Ponds of Haematococcus pluvialis

A 200,000 liter outdoor pond is inoculated with Haematococcus pluvialis at an initial OD (750 nm) of ˜0.15 (pond P08). Growth of the microalgae is monitored daily by measuring the ash free dry weight of the culture. PCR monitoring is performed daily to detect the presence of the chytrid fungus Paraphysoderma sedebokerensis or close relatives using ACCTTCATGCTCTTCACTGAGTGTGATGG (SEQ ID NO. 38) and TCGGTCCTAGAAACCAACAAAATAGAAC (SEQ ID NO. 39) as primers.

The pond is further monitored using fluorescent dye binding assays as described in Example 4 above. Samples are also examined for fungal contamination using Solapehnyl flavine fluorescent dye staining using the same protocol. Solaphenyl flavine staining is measured using an excitation wavelength of 365 nm and emission is detected at 515 nm. Microscopic examination is performed using a FITC filter.

Samples are further examined for the growth of microalgae by detecting chlorophyll fluorescence. Chlorophyll fluorescence is measured using an excitation wavelength of 430 nm and an emission wavelength of 685 nm. The results of microalgae growth is used to prepare a semi-log plot of chlorophyll fluorescence versus time to identify growth phases and prepare harvest schedules.

The health of a microalgae pond is further evaluated using a flocculation assay. Samples are obtained from the growing pond and placed in 17×100 mm culture tubes. 200 μl samples are taken from the same depth of the tube at T₀ and at 40 minutes. The settling rate is determined as the ratio of OD750 or the chlorophyll fluorscence at T₄₀/T₀. Ponds are monitored using a FlowCAM. FlowCAM analysis integrates flow cytometry and microscopy allowing for high-throughput analysis of particles in a moving field. Diluted (1:10) culture samples are run through the FlowCAM with a 20× objective (green algae) or a 4× objective (blue-green algae). The FlowCAM and its integrated software automatically images, counts, and analyzes a predetermined amount of particles (typically 3,000) in a continuous flow. Phenotypic attributes (e.g. green vs. transparent cells, large cells vs. small cells, etc.) are recorded.

Initially, the Cₜ value for the pest is above the threshold of Cₜ=30. The measured Cₜ observed for P. sedebokerensis stays below Cₜ=30 and indicates a need for crop protective action. Once detected with a Cₜ<30, chlorothalonil is added at a concentration of 1 ppm. Daily monitoring is continued and the Cₜ observed for P. sedebokerensis increases above the Cₜ=30 threshold and remains above the threshold.

### Example 12

### Monitoring and Treatment of Ponds of Arthrospira

A 200,000 liter outdoor pond located in Las Cruces, N. Mex. is inoculated with Arthrospira sp. at an initial DW of 0.2 g/l. Growth of the microalgae is monitored daily by measuring the ash free dry weight of the culture. qPCR monitoring is performed daily to detect the presence of the chytrid fungus Rhizophidium planktonicum or close relatives using primers CCGTGAGGGAAAGATGAAAA (SEQ ID NO. 40) and CCTTGCGCTTTTTACTCCAG (SEQ ID NO. 41).

The pond is further monitored using fluorescent dye binding assays as described in Example 4 above. Samples are also examined for fungal contamination using Solapehnyl flavine fluorescent dye staining using the same protocol. Solaphenyl flavine staining is measured using an excitation wavelength of 365 nm and emission is detected at 515 nm. Microscopic examination is performed using a FITC filter.

Samples are further examined for the growth of microalgae by detecting chlorophyll fluorescence. Chlorophyll fluorescence in a cyanobacteria culture is measured using an excitation wavelength of 363 nm and an emission wavelength of 685 nm. The results of microalgae growth is used to prepare a semi-log plot of chlorophyll fluorescence versus time to identify growth phases and prepare harvest schedules.

Ponds are monitored using a FlowCAM. FlowCAM analysis integrates flow cytometry and microscopy allowing for high-throughput analysis of particles in a moving field. Diluted (1:10) culture samples are run through the FlowCAM with a 4× objective (blue-green algae). The FlowCAM and its integrated software automatically images, counts, and analyzes a predetermined amount of particles (typically 3,000) in a continuous flow. Phenotypic attributes (e.g. green vs. transparent cells, large cells vs. small cells, etc.) are recorded.

Initially, the Cₜ value for the pest is above the threshold of Cₜ=30. The measured Cₜ observed for R. planktonicum stays below Cₜ=30 and indicates a need for crop protective action. Once detected with a Cₜ<30, chlorothalonil is added at a concentration of 1 ppm. Daily monitoring is continued and the Cₜ observed for R. planktonicum increases above the Cₜ=30 threshold and remains above the threshold.

While the invention has been described, it will be understood by those skilled in the art that various changes may be made to adapt to particular situations without departing from the scope of the invention. Therefore, it is intended that the invention not be limited to the particular embodiments disclosed for carrying out this invention, but that the invention will include all embodiments falling within the scope and spirit of the appended claims.

1. A method of reducing the growth of a fungus in a liquid system comprising:
inoculating said liquid culture with a microalgae;
detecting said fungus;
providing an effective concentration of fungicide selected from the group consisting of fluazinam, pyraclostrobin, thiram, chlorothalonil, dithianon, dodine, and dibromocyanoacetamide to inhibit the growth of said fungus relative to the growth of said fungus without said fungicide; and
growing said microalgae.

2. (canceled)

3. The method of claim 1, wherein said fungicide is selected from the group consisting of fluazinam, pyraclostrobin and thiram.

4. The method of claim 1, further providing a second effective concentration of a fungicide selected from the group consisting of fluazinam, pyraclostrobin, thiram, chlorothalonil, dithianon, dodine, and dibromocyanoacetamide.

5. The method of claim 1, further providing a second effective concentration of a fungicide selected from the group consisting of fluazinam, pyraclostrobin and thiram.

6. (canceled)

7. The method of claim 1, wherein said fungus is a member of the Chytridiomycota division of the fungi kingdom.

8. The method of claim 7, wherein said member of the Chytridiomycota division of the fungi kingdom is selected from the group consisting of Chytridiales, Rhizophylctidales, Spizellomycetales, Rhizophydiales, Lobulomycetales, Cladochytriales, Polychytrium and Monoblepharidomycetes.

9. The method of claim 1, wherein said microalgae is selected from the group consisting of nannochloropsis, desmodesmus, scenedesmus and spirulina.

10. (canceled)

11. (canceled)

12. (canceled)

13. The method of claim 1, wherein said growing provides a yield of microalgae that is at least 80%, at least 85%, at least 90%, at least 95%, at least 97.5%, at least 99% or 100% of the yield of microalgae harvested from a liquid culture of microalgae that has not been provided a fungicide.

14. (canceled)

15. The method of claim 1, wherein said growing provides a yield of microalgae that is at least 10%, least 15%, at least 20%, at least 25%, at least 50%, at least 75% or 100% greater than the yield of microalgae harvested from a liquid culture of microalgae having said fungus and that has not been provided said fungicide.

16. (canceled)

17. The method of claim 1, wherein said yield is selected from the group consisting of at least 1.5 fold, 2.0 fold, 2.5 fold, 5.0 fold, 7.5 fold, 10 fold and 15 fold greater than the yield of microalgae harvested from a liquid culture of microalgae having said fungus and that has not been provided a fungicide.

18. (canceled)

19. (canceled)

20. (canceled)

21. The method of claim 1, wherein said liquid system is an open outdoor culture system.

22. (canceled)

23. (canceled)

24. The method of claim 21, wherein said liquid system is a continuous culture system.

25. (canceled)

26. Method of detecting the presence of a fungus in a liquid culture system of microalgae comprising:
obtaining a sample of said liquid culture system; and
detecting the presence of a DNA sequence of said fungus.

27. The method of claim 26, wherein said fungus is a member of the Chytridiomycota division of the fungi kingdom.

28. The method of claim 27, wherein said member of the Chytridiomycota division of the fungi kingdom is selected from the group consisting of Chytridiales, Rhizophylctidales, Spizellomycetales, Rhizophydiales, Lobulomycetales, Cladochytriales, Polychytrium and Monoblepharidomycetes.

29. The method of claim 26, wherein said DNA sequence is a ribosomal DNA sequence selected from the group consisting of NC_{—}003053 Rhizophydium sp. 136 mitochondrion, NC_{—}003048 Hyaloraphidium curvatum mitochondrion, NC_{—}003052 Spizellomyces punctatus mitochondrion chromosome 1, NC_{—}003061 Spizellomyces punctatus mitochondrion chromosome 2, NC_{—}003060 Spizellomyces punctatus mitochondrion chromosome 3, NC_{—}004760 Harpochytrium sp. JEL94 mitochondrion, NC_{—}004624 Monoblepharella sp. JEL15 mitochondrion, and NC_{—}004623 Harpochytrium sp. JEL105 mitochondrion or a sequence selected from the group consisting of SEQ ID NOs: 1 to 6.

30. A method of growing microalgae in a liquid system, comprising:
inoculating said liquid system with a microalgae;
growing said microalgae in said liquid system for at least 10 days after said inoculation;
monitoring said liquid system at least once for the presence of a fungus, wherein said monitoring can detect said fungus at a level of at least 10⁶ cells per milliliter (cells/ml);
detecting the presence of said fungus;
providing to said liquid system an effective concentration of a fungicide selected from the group consisting of fluazinam, pyraclostrobin, thiram, chlorothalonil, dithianon, dodine, and dibromocyanoacetamide and
harvesting microalgae from at least a part of said liquid system.

31. The method of claim 30, wherein said fungus is a member of the Chytridiomycota division of the fungi kingdom.

32. The method of claim 31, said member of the Chytridiomycota division of the fungi kingdom is selected from the group consisting of Chytridiales, Rhizophylctidales, Spizellomycetales, Rhizophydiales, Lobulomycetales, Cladochytriales, Polychytrium and Monoblepharidomycetes.

33. (canceled)

34. (canceled)

35. The method of claim 30, wherein said fungicide is an effective concentration of a fungicide selected from the group consisting of fluazinam, pyraclostrobin and thiram.

36. The method of claim 30, wherein said growing days is selected from the group consisting of 15 or more, 30 or more, 45 or more, 60 or more, 90 or more, 120 or more, 180 or more, 250 or more, 500 or more, 1000 or more, 1500 or more and 2000 or more days after said inoculation.

37. (canceled)

38. (canceled)

39. The method of claim 30, wherein said monitoring is capable of detecting said fungus at a level selected from the group consisting of at least 10⁵ cells/ml, 10⁴ cells/ml, 10³ cells/ml, 10² cells/ml, and 10¹ cells/ml.

40-50. (canceled)

51. The method of claim 30, wherein said liquid system is an open outdoor system.

52. (canceled)

53. (canceled)

54. The method of claim 51, wherein said liquid system is a continuous culture system.

55-65. (canceled)

66. The method of claim 30, wherein said microalgae is selected from the group consisting of nannochloropsis, and spirulina.

67-149. (canceled)


SEQUENCE LISTING (41 sequences)

SEQ ID NO: 1 - DNA, 2462 bp, unknown
  Organism is a member of the fungi kingdom and is a new member of the class chytridomycetes.
aagtaaaagtcgtaacaaggtttccgtaggtgaacctgcggaaggatcattcttgtatcactgtgtgtgtgtgtatctatttgtacacatactctctgtatagaatatcatttctgtgatattttatttattaattatgtgaaatgtgccagtctacttttgctttgcggcgacgtagtgtgtgaagcaatgtgtttctgttagcttgctgtctcatcgtctgcacgcactctttcgggggtgttgctgtggtgatgaatgtacagcttgtggacagttgcacatggtcagatatttactgtctgattccacgctttgtcgcaagcagatgatgtagacaatccattgaaatttaattttttactacaacaactacctactgtcaaagtcagaaataaatttaaaaattattatgacaacttttagcaacggatctctaggctctcgcaacgatgaagaacgcagcaaactgcgaaacgtagtgtgaattgcagaccgtgaatcatcgaatctttgaacgcactttgcgctgtctggaacactggacagcatgtctgtttgagcatcatttctatacatcctgttttctattacctgcgttatgcgtgtgctgggtgttcatcatgtgcaactaactttgttgttgatttgtacctggtggtagcactcatcatgcgtgcagtgtgattgtaacagggatttgtggtttatcttggttacttgtgtacatgcttcttttcttcttggagagaagttactgtcgcttgtatatgatattccattaaatatactgaagattctccatacatgatttattcttgtatgctcttcgaaagacgtatgtcctttgtgaacactatcttttaaagaaagagaagaatcttttgtaaagaatctctcatacaggttcttcatcgaatacattatgtgtcagcgtagtcactttttgtgtagctgcctggaaacattggtgtgtttggaaaaccgatctcaaatcagacaagattacccgctgaacttaagcatattaataagcggaggaaaagaaactaacaaggattcccatagtaacggcgagtgaagtgggaacagctcaaatttgtaatctcttcggagagttgtaatttgtagaggcgttttcgacggttaaccgggtagaagtctcttgggaaagagcgtcacagagggtgagaatcccgttcgtgatccgggtataccgcagatatgatacgctttcaaagagtcgggttgtttgggactgcagccctaaattggtggtatattccatctaaagctaaatacaggcgagagaccgatagcgaacaagtactgtgaaggaaagatgaaaagaactctgaagagagagttaaaagtacgtgaaattgctaaaagggaaacgtttgaaatcagtgcagcctggatgaagttcagccattcgtaccgggtggtgtatgcttcgttcggccggtcagcatcggttcaataaaacatttatgtgccagaaggagaacccgcgtggaaggtagctcttcttcgggagagtgttacagacacgcggaaaacttctggctggaccgaggaaagccttgtgcaggatgctgacgaaatggtttcaaatgacccgtcttgaaacacggaccaaggagtctaacacatatgcgagtatttgggattcactctcgaatgcgcaatgaaagtgaacgtaggtgggacctttcgggggcaccatcgaccgatctggatgttttatattgatagatttgagtaagagcatatttgttgggacccgaaagatggtgaactatgcctgaatagggcgaagccagaggaaactctggtggaggctcgtagcgattctgacgtgcaaatcgatcgtcaaatttgggtataggggcgaaagactaatcgaactatctagtagctggttccctccgaagtttccctcaggatagcagagatttatatacatcagttagatcaggtaaagcgaatgattagaggccttggggttgaaacaaccttaacctattctcaaactttaaataggtctgaagtctatgttacttaattgaacgtagacactcgaatgataatctctagtgggccatttttggtaagcagaactggcgatgcgggatgaaccgaacgtcgagttaaggtgccggaatacacgctcatttagatcccataaaaggtgttagttcatctagacagcaggacggtggccatggaagtcggaatccgctaaggagtgtgtaacaactcacctgccgaatgaactagccctgaaaatggatggcgcttaagcgtgttacccatactcgaccatcagggtaaatgcgaaactttgatgagtaggagggcgtagaggtcgtgaagaagtctagacagtgatgtcggatgaaacggcctctagtgcagatcttggtggtagtaaagggcgaattctgcagatatccaat

SEQ ID NO: 2 - DNA, 4078 bp, unknown
  Organism is a member of the fungi kingdom and is a new member of the class chytridomycetes.
cgccagttgtgatggatatctgcagaattcgcccttaacctggttgatcctgccagtagtcatatgcttgtctcaaagattaagccatgcatgtctaagtataagcactttatactgtgaaactgcgaatggctcattaaatcagttatagtttatttgatagtaccttactacttggataaccgtggtaattctagagctaatacatgctaaaaatcccgacttctggaagggatgtatttattagataaaaaaccaacccgggcaaccggttctttggtgattcataataacttttcgaatcgcatggctttacgccggcgatggttcattcaaatttctgccctatcaactttcgatggtaggatagaggcctaccatggtggtaacgggtaacggggaattagggttcgattccggagagggagcctgagaaacggctaccacatccaaggaaggcagcaggcgcgcaaattacccaatcctgacacagggaggtagtgacaataaataacaatgcagagctcttcgagttttgcaattggaatgagtacaatttaaatcccttaacgaggaacaattggagggcaagtctggtgccagcagccgcggtaattccagctccaatagcgtatattaaagttgttgcagttaaaaagctcgtagttgaattttgggcttggttgggtggtctgccgattggtatgtactactcgactgggtcttaccttctagggaaccttcatgctcttcactgagtgtgatggggatctagaactkttactgtgaraaaattagagtgttyaaagcaggcttacgcywgaatacwttagcatggaataayamrataggactytggytctattttgttggtytstaggaccgragtaatgrttaakagggayagtygggggcattmgtatttmrttgtcagaggtgaaattcttggatttaygaaagackaactwctgcgaaagcatttgccaaggatgttttcattaatcaagaacgaaagtyrggggmtcgaarasgattagayaccstygtagtctyraccataaackatgccgactmgggattgggcgaatgttwtttywtgactcgccagcaccktatgagaaatcaaagtytttgggttcyggggggagtatggtcgcaaggctgaaacttaaaggaattgacggaagggcaccaccaggagtggagcctgcggcttaatttgactcaacacggggaaactcaccaggtccagacatagtaaggattgacagattgagagctctttcttgattctatgggtggtggtgcatggccgttcttagttggtggagtgatttgtctggttaattccgataacgaacgagaccttaacctgctaaatagtcacactaaccttgtgttggtggctgacttcttagagggactattgacgtttagtcaatggaagtttgaggcaataacaggtctgtgatgcccttagatgttctgggccgcacgcgcgctacactgatgaagtcaacgagtttataaccttggccggaaggtctgggtaatcttgtgaaacttcatcgtgctggggatagaccattgcaattattggtcttcaacgaggaattcctagtaagcgtgagtcatcagctcgcgttgattacgtccctgccctttgtacacaccgcccgtcgctactaccgattgaatggcttagtgaggcttctggattggcaggtcgatgctggcaacagcgctgaccagctgagaagttagtyaaacttggtcatytagaggaagtaraagtcgtaacaaggtttccgtaggtgaacctgcggaaggatcattactaagttwtaaacmacwtasccaaacattgtgaactgtttacccctcttggaacttktarttctwckagsgmsctkatkcrmswaacctwtgwttatwkatsaktkrcktttkgmatrcytaamwycagwgctaaccactktcaraaccrawctctaagctcttgmwtckatkaactggcaatckyaacaaagacaactctcawcwrcrgatatctyggcyctygcatcgatgaagaacgcagcgaaatgcgataagtaatgtgaattgcagaactccgtgaatcatcgagtctttgaacgcatattgcaccttggggtattccccaaggtatgcctgtttgagtgtcagtaactaatctcaaccccttacgggtgttggagtgtggacttctttctttctccaaagagagttgtcttaaatgtattggtgagcggttttgatctgaagtagatctgctctaactttgataaagtaaactttacttgttctagttaccggatcttcttgttaacagttgaagaatcgttttcaccttacttgttacaacagactaagagtttagtcgagtcttcacggacgcgtcttagctctttttttgttcacttgacctcaaatcaggtaggactacccgctgaacttaagcatattaataagcggaggaaaagaaactaacaaggattcccctagtaacggcgagtgaagcgggaagagctcaaatttgaaatctgggaggttttacctctccgaattgtagtttgtagaagcgtcatctgcgggtgctagagtttaagtcccttggaagagggcgtcatagagggtgagaatcccgtctataactctagtcattctgctctgtgttgcgctttctaagagtcgggttgtttgggattgcagcccaaaatgggtggtaaatttcatccaaagctaaatattggcaagagaccgatagcgaacaagtaccgtgagggaaagatgaaaagaactttgaaaagagagttaaaagtacgtgaaattgctgaaagggaaacgcttgaaaccgttccattctagtagaagatcagttagttgactcggattgcattggtttggtgaggttaaaccaccggctagtgtttctttgagctctgctgatgcactcttctgctggtagatcaacatcagtttactctgctggaaaaaagctaggagaaggtagcttgggcttcggttcaagtgttatagctcctagtccatacagtgaggtagactgaggactgctgtgcatgcctttggctgggtctctgcaggccaagtgatttgcatctgctggctgcttgcagactgtgggtgacacgttacttgctcttgtagtcagactacaaatgcacataggatgttgatgaaatggttttaatcgacccgtcttgaaacacggaccaaggagtctaccaagtgcgcgagtgtttgggtgaaaaacccatacgcggaatgaaagtaatttcgatgggatcctgtaaaggtgcaccatcgaccgggccttgagctactgcgacaggtctgagtatgagcgtatttgttaggacccgaaagatggtgaactatgcctgaatagggtgaagccagaggaaactctggtggaggctcgtagcgattctgacgtgcaaatcgatcgtcaaatttgggtataggggcgaaagactaatcgaaccatctagtagctggtttccgccgaagtttccctcaggatagcagagactcgatactcagttttatgaggtaaagcgaatgattagaggccttggggttgaaacaaccttaacctattctcaaactttaaatatgtaagaagtccaagttacttaattgaacttggacatcgaatgtgagtctctagtgggccatttttggtaagcagaactggcgatgcgggatgaaccgaacgttgagttaaggtgccggaatacacgctcatttagacaccacaaaaggtgttagttcatctagacagcaggacggtggccatggaagtcggaatccgctaaggagtgtgtaacaactcacctgccgaatgaactagccctgaaaatggatggcgcttaagcgtgttacctatactcgaccgttaaggtaaatgcgatgccttaacgagtaggcggacgtggaggtagtgaagaagctttgggcgtgagcctgagtgaaactgcctctagtgcagatcttggtggtagtaaagggcgaattctgcagatatccatcacac

SEQ ID NO: 3 - DNA, 2758 bp, unknown
  Organism is a member of the fungi kingdom and is a new member of the class chytridomycetes.
gccagtgtgatggatatctgcagaattcgcccttaacttaaaggaattgacggaagagcacaacaaggcgtggaatatgcggcttaatttgactcaacacggggaaacttaccaggtccagacttaataaggattgacagattgagagctctttcttgattttaaggggtgtggtgcatggccgttcttagttggtggagtgatttgtctgcttaattgcgataacgaacgagaccttttcctgctaaatagactcactcagccttgctgggtgcagccttcttagagggacttttgatgtttaatcaaaggaagcttgaggcaataacaggtctgtgatgcccttagatgttctgggccgcacgcgcgttacactgatgaagtcaacgagtttattccttggctgaaaagtctgggtaatctttttaaacttcatcgtgctggggatagagcattgcaattattgctcttcaacgaggaatgcctagtaagcgcaagtcatcagcttgcgttgattacgtccctgctctttgtacacaccgcccgtcgctactaccgatgaatggcttagtgaggccttgggattgagtgtctgtctggcaacagacgtgtcacttgagaacttggtcaaacttggtcatttagaggaagtaaaagtcgtaacaaggtatccgtaggtgaacctgcggatggatcattactaaatctgtcgtgcgtatgactctgcattcttgaatgcgtctcattccacttcaaattactcatcattgtgaactgttgctaatgtgtatgcaatcacctttttggtgacgcgtgccattagtcttttattaaactaaaacttttaaactaaagatctctgcacaaacttagtgttgtgtcaactaaaaactaaaacttttagcaacggatctctaggctctcgcatcgatgaagaacgcagcgaattgcgaaatgtaatgtgaattgcatttaccgcgaatcattaaatctttgaatgcactttgcactgctgtgwattcacggcagtacgcctgtttgagcatcaatcaaatttctcacttttatatagtgattgtgaagtactggatacagtacttttgagtttacacaaactgttcgtgtygatacaacagttggcaatatctatatcgatcttgtcaactctaatcaaccgtacgcgtgtttattgagctctgactgctctctcattacattgatctcaaatcaggcgagaatacccgctgaacttaagcatattaataagcggaggaaaagaaactaacaaggattccctcagtaactgcgagtgaagcgggaccagctcaaatttaaaatctcgcaagagaattgtagtttagagaagtgtacacgatgacgcggcgcgttaaagtctcctggaatggagcgtcatagagggtgagaatcccgttgataatgcgccgtcatgtcacaattcgttgcatcttccaagagtcggattgtttgggaatgcagtccaaaatgggtggtatatttcatccaaagctaaataccggcgagagaccgatagcgaacaagtaccgtgagggaaagatgaaaagaactttgaaaagaaagttaaacagtacgtgaaattgctaaaagggaaacatttgcaatcagtgttgcggcagtcaagcaattggctgtcgtgggtcaatatttacgcgtggtgaagtagatcagttcgagaaggtgactgtcacttcggtgtcagtgttatagctcgggcgaccacttcattgcgcgtaaaggcatgcaatgtattttggatgaacactgtcctgtttgagagtagattggtaaactgcgtgcagattgcttttcgatttcaggcatgatggtggtagtactcatacattcaggatattgacgaaatggttgtaaatgacccgtcttgaaacacggaccaaggagtctaacaaatatgcaagtgttagagttgtaaactctggcgcgaaatgaaagtgaacgttggtgggatctctcacgagtgcaccatcgaccgatctggatttttaatgaaagatttgagtctgagcatatttgctgggacccgaaagatggtgaactatgcctgaatagggtgaagccagaggaaactctggtggaagctcgtagcggtactgacgtgcaaatcgttcgtcaaatttgggtataggggcgaaagactaatcgaaccatctagtagctggttccctccgaagtttctctcaggatagctgagactgttacgcagttagatcaggtaaagcgaatgattagaggccttggggaattaaacttcctcaacctattctcaaactttaaataggtctgaagtcaacgttacttaactgaacgtttgacattcgaatgcaagtctcaagtgggccatttttggtaagcagaactggcgatgcgggatgaaccgaacgtcgagttaaggtgccggaatacacgctcatttagatcccataaaagttgtcggtacatctagacagcaggacggtggtcatggaagttgaaatccgctaaggagtgtgtaacaactcacctgccgaatgtaccgacgctgaaaatggatggcgcttaagcgtgttacccatactcgaccgtcaagggcagcacctttgacgagtaggagggcgtgaaggttgtgaagaagtctagactgtgaagtcggattgaacagcctttagtgcagatcttggtggtagtaaagggcgaattctgcagatatccatcacactggcgg

SEQ ID NO: 4 - DNA, 3293 bp, unknown
  Organism is a member of the fungi kingdom and is a new member of the class chytridomycetes.
awcctggttgatcctgccagtagtcatatgcttgtctcaaagattaagccatgcatgtctaagtataaacaattttgtactgtgaaactgcgaatggctcattaaatcagttatagtttatttgataataccttactacttggataaccgtggtaattctagagctaatacatgctacttcacccgacttctggaagggtggtactgattagatacaaaaccaacccgggcaaccggtttattggtgattcatagtcatttctcgaatcgtatgactttacgtcgacgatggttcattcaaatttctgccctatcaactttcgatggtaggatagaggcctaccatggttttaacgggtaacggagaattagggttcgattccggagagggagcctgagaaacggctaccacatccaaggaaggcagcaggcgcgcaaattacccaatcccgacacggggaggtagtgacaataaataacaatgcagagccctatgggttttgtaattggaatgagtacaatttaaatcccttaacgaggaacaattggagggcaagtctggtgccagcagccgcggtaattccagctccaatagcgtatattaaagttgttgcagttaaaaagctcgtagttgaattttgggcccagctgagtggtctagcttaacggttagcactgctttggttgggcctttccttctggctagccagtgtgctcttaattgggtgcgctggggaaccaggacttttactgtgaaaaaattagagtgtttaaagcaggcttacgcttgaatacattagcatggaataatagaataggactttggttctattttgttggtttctaggaccgaagtaatgattaatagggatagttggggggcattagtaattktgaayttktcvtgcwggtgatarttcattgkaattttgactgaacgaggaattcctagtaagcktgagtcatcagctcgcgttgattacgtccctgccctttgtacacaccgcccgtcgttactaccgattgaatggcttagtgagacctccggattggcgatctgtgactggaaacagaaacgggttgctgagaagctggtcaaacttggtcatttagaggaagtacaagtcgtaacaaggtawccgtaggtgaacctgcggttggatcattactaatgggtttaaatacctaatacatttgtgtgaactagataaaaattgtacttggtgagtaggatgtttcggcatccagtggcttcactttttcgtttcgaaaatgtttggagcaactcaccgccatttttaatcaacctattttataaattgagacttgaaattcttttgttaaaaactaaagaaacaacttttgacaacggatctcttggctctcgcaacgatgaagaacgcagcgaaatgcgatacgtaatgtgaattgcagaattcagtgaatcatcgaatctttgaacgcatattgcgctttctggtattccggagagcatgcctgtttgagtaccttttaatttctcaaatctcatgtatgtgagaatttggagtctgagttgatctgtttttataagtgaaagcagcagcttgaaacgtattccatgtagtagtttttgcggtagcgaaaatagtagttggaaaagaattcgaatgagattcctatctcagctttttctaagtacactattattgtcgaagcttgtacataagctacaatgtcattgggattgcctctatgtttgtccataagaacaaatttggaagyaaatgtttttaaatcttggtctcaaatcaggtaggaggacccgctgaacttaagcatatcaataagcggaggaaaakaaactaaccaggattcccctagtaacggcgagtgaagcgggaatagctcaaatttgaaatctcacaggtttcctgtgcgaattgtagtttaaagagtcgttttcgagggctgcttgagtacaagtcctttggaatagggcatcatagagggtgaraatcccgtctttgactcaaggctagttccttgtgtgatgcracttcmaagartcgtttgtttgggatgcaraccaaaatgggtggtaaataccatctaaggctaawtatggcgagagaccgatagcgaacmagtaccgtgagggaaaggatgaaaagaactttgaaaagagagttaaacagtacgtgaaattgtcaaaagggaaacgcttgaaaccagtgttagwttttgaggaatcagtcgttctttggacgatgcaytttctcaagaactggtcacatcgattggattgaggtaaaatttttctgatgtagcccttgggtgttatagacgaagagatscttggtttggatcgaggtytgcagcatttttggggagtcagttcgccttttgaagcgtcaaatcgcacagcatgctgagtgctttgaatgccagaaagatcgggctgttcctccacacttgtgcttaggatgttgacaaaatggttttaaacgacccgtcttgaaacacggaccaaggagtctaacatgtatgcgagtatttgggtggcaaaccctttatgcgtaatgaaagtgaaacgggtgggatacttgcaccatcgaccggcctggatttctttgaaaggtctgagtaagagcatatatgctgggacccgaaagatggtgaactatgcctgaatagggtgaagccagaggaaactctggtggaggctcgtagcgattctgacgtgcaaatcgatcgtcaaatttgggtataggggcgaaagactaatcgaaccatctagtagctggttccctccgaagtttccctcaggatagcagaagctcatatcagttttatgaggtaaagcgaatgattagaggccttggggatatattatccttaacctattctcaaactttaaatacgtaagaagtttaggttacttaattgaacctagacattgaatgagagcttctagtgggccatttttggtaagcagaactggcgatgcgggatgaaccgaacgttgagttaaggtgccggaatacacgctcatttcgataccacaaaaggtgttagttcatctagacagcaggacggtggccatggaagtcggaatccgctaaggagtgtgtaacaactcacctgccgaatgaactagccctgaaaatggatggcgctcaagcgtgttacccatactcaaccgtcagggtaaaacgaggctctgacgagtaggagggcgtggaggtttgtgaagaagccttagacgtgagtctgggtgaaacagcctctagtgcagatcttggtggtagtaa

SEQ ID NO: 5 - DNA, 796 bp, unknown
  Organism is a member of the fungi kingdom and is a new member of the class chytridomycetes.
acagctatgaccatgattatgcgaagctatttaggtgacactatagsatactcaagctatgcatcaagcttggtaccgagctbggatccactmgtaactggccgccagtgtgcyggaacttcgccctttcctccgcttattgatatgcttaagttcagcgggtagtcctacctgatttgagaccaaggttcaaagagttgtaaacaaccctttataaggttatgaactgccttgaaagataccactcccctatacaaataacttaattagtatcatagagaaaccaaggtttcagtcaaatacttttctgttattatatagtaaaggatcaacatagtaccctatacaaaacagtaatgaatttcaaagtactctgggtagagacacttcaaatctttacagtgatactaggtaaaaccacatatcaaggtaaaagaaatacattagattctcaaacaggcatactctaaaagagtgcaatgtgcgttcaaagatttgatgattcacggaattctgcaattcacattacgtatcgcatttcgctgcgttcttcatcgttgcgagagccaagagatccgttgtcaaaagttgtttttgtttactatataaacaagtcagtcaatttaaaacagtggtttaatataatgctgggtacactctattactagagcctacccaaaagacattgaattgcacaaagtgtgaaagagtagtacattagtgagctcaactaggagcatcaaccgcagtaaaactcataaatcagtaatgatccaaccgcaggttcacctacgga

SEQ ID NO: 6 - DNA, 1799 bp, unknown
  Organism is a member of the fungi kingdom and is a new member of the class chytridomycetes.
cttaaaggaattgacggaagggcaccaccaggagtggagcctgcggcttaatttgactcaacacggggaaactcaccaggtccagacataataaggattgacagattgagagctctttcatgattttatgggtggtggtgcatggccgttcttagttggtggagtgatttgtctggttaattccgataacgaacgagaccttaacctgctaaatagttgcatttactctggtagatgatcaacttcttagagggactataggcgactagtctatggaagtttgaggcaataacaggtctgtgatgcccttagatgttctgggccgcacgcgcgctacactgataaaggcaacgagtaattcaccttggccggaaggtctgggtaatcttgtgaaactttatcgtgctggggatagtcctttgcaattattggacttaaacgaggaattcctagtaagcgtgagtcatcagctcgcgttgattacgtccctgccctttgtacacaccgcccgtcgctagtaccgattgaatggcttagtgagacctccggattgatagttcattggtggcaacaccatagaatcgttgagaagctggtcaaacttggtcatttagaggaactaaaagtcgtaacaaggtaaccgtaggtgaacctgcggttggatcattactgatttatgagttttactgcggttgatgctcctagttgagctcactaatgtactactctttcacactttgtgcaatkcmatgtstkwtggrtagkctsyagwawtmgmsyktacccagcattgtattaaaccactgttttaaattgactgacttgtttatatagtaaacaaaaacaacttttgacaacggatctcttggctctcgcaacgatgaagaacgcagcgaaatgcgatacgtaatgtgaattgcagaattccgtgaatcatcaaatctttgaacgcacattgcactcttttagagtatgcctgtttgagaatctaatgtatttcttttaccttgatatgtggttttacctagtatcactgtaaagatttgaagtgtctctatcaagggcgaattctgcagattcattactgttttgtatagggtactatgttgatcctttactatataataacagaaaagtatttgactgaaaccttggtttctctatgatactaattaagttatttgtataggggagtggtatctttcaaggcagttcataaccttataaagggttgtttacaactctttgaaccttggtctcaaatcaggtaggactacccgctgaacttaagcatatcaataagcggaggaaaagaaactaacaaggattcccctagtaacggcgagtgaagcgggaacagctcaaatttgaaatctcacctctggtgcgaattgtagtttagagaaacgttttcgggcttgacggtaggtacaagttcgctggaatgcgatatcatagagggtgataatcccgtctttgacctattgttcaagtccatatgatacgttttcgaagagtcggattgtttgggaatgcagtccaaaatgggtggtaaataccatctaaagctaaatattggcgagagaccgatagcgaacaagtaccgtgagggaaagatgaaaagaactttgaaaagagagttaaacagtacgtgaaattgtcaaaagggaaacgtttgaacccagtattgtatgcagaaatttagctgtgatttattgcagtgtccttttctgtttacagtcaacatcagtttgatttggagtacaagctagaggagnggccttcgggtgtatagcctc

SEQ ID NO: 7 - DNA, 20 bp, Artificial Sequence
  Peptide nucleic acid.
gcaagctggtgcgagtaatt

SEQ ID NO: 8 - DNA, 20 bp, Artificial Sequence
  Peptide nucleic acid.
gagtgaatccgattgggaga

SEQ ID NO: 9 - DNA, 20 bp, Artificial Sequence
  Peptide nucleic acid.
gtcactgggatatcgtcgct

SEQ ID NO: 10 - DNA, 19 bp, Artificial Sequence
  Primer
tccgtaggtgaacctgcgg

SEQ ID NO: 11 - DNA, 20 bp, Artificial Sequence
  Primer
tcctccgcttattgatatgc

SEQ ID NO: 12 - DNA, 20 bp, Artificial Sequence
  Primer
gctgcgttcttcatcgatgc

SEQ ID NO: 13 - DNA, 20 bp, Artificial Sequence
  Primer
gcatcgatgaagaacgcagc

SEQ ID NO: 14 - DNA, 21 bp, Artificial Sequence
  Primer
aacctggttgatcctgccagt

SEQ ID NO: 15 - DNA, 16 bp, Artificial Sequence
  Primer
gggcatcacagacctg

SEQ ID NO: 16 - DNA, 19 bp, Artificial Sequence
  Primer
gtacccgctgaacttaagc

SEQ ID NO: 17 - DNA, 17 bp, Artificial Sequence
  Primer
tactaccaccaagatct

SEQ ID NO: 18 - DNA, 18 bp, Artificial Sequence
  Primer
tagatacccyggtagtcc

SEQ ID NO: 19 - DNA, 17 bp, Artificial Sequence
  Primer
aaggaggtgwtccarcc

SEQ ID NO: 20 - DNA, 23 bp, Artificial Sequence
  Primer
cgacgttgtaaaacgacggccag

SEQ ID NO: 21 - DNA, 28 bp, Artificial Sequence
  Primer
cacaggaaacagctatgaccatgattac

SEQ ID NO: 22 - DNA, 20 bp, Artificial Sequence
  Primer
cacgcgtacggttgattaga

SEQ ID NO: 23 - DNA, 20 bp, Artificial Sequence
  Primer
tgaatgcactttgcactgct

SEQ ID NO: 24 - DNA, 22 bp, Artificial Sequence
  Primer
ccacaaatccctgttacaatca

SEQ ID NO: 25 - DNA, 20 bp, Artificial Sequence
  Primer
ttacctgcgttatgcgtgtg

SEQ ID NO: 26 - DNA, 20 bp, Artificial Sequence
  Primer
gatcaaaaccgctcaccaat

SEQ ID NO: 27 - DNA, 20 bp, Artificial Sequence
  Primer
tgaattgcagaactccgtga

SEQ ID NO: 28 - DNA, 20 bp, Artificial Sequence
  Primer
atgtcattgggattgcctct

SEQ ID NO: 29 - DNA, 20 bp, Artificial Sequence
  Primer
cgggtcctcctacctgattt

SEQ ID NO: 30 - DNA, 20 bp, Artificial Sequence
  Primer
gggcgtaccataatctgcat

SEQ ID NO: 31 - DNA, 20 bp, Artificial Sequence
  Primer
atgacaccgtcaggaaaacg

SEQ ID NO: 32 - DNA, 19 bp, Artificial Sequence
  Primer
cggaccaaggagtctaaca

SEQ ID NO: 33 - DNA, 20 bp, Artificial Sequence
  Primer
ttgcacgtcagaatcgctac

SEQ ID NO: 34 - DNA, 20 bp, Artificial Sequence
  Primer
taccctcacccctctctcct

SEQ ID NO: 35 - DNA, 20 bp, Artificial Sequence
  Primer
taagcttcagccaacccaat

SEQ ID NO: 36 - DNA, 20 bp, Artificial Sequence
  Primer
ctgggatatcgtcgctccta

SEQ ID NO: 37 - DNA, 20 bp, Artificial Sequence
  Primer
atgggtatgcgtccgttaga

SEQ ID NO: 38 - DNA, 29 bp, Artificial Sequence
  Primer
accttcatgctcttcactgagtgtgatgg

SEQ ID NO: 39 - DNA, 28 bp, Artificial Sequence
  Primer
tcggtcctagaaaccaacaaaatagaac

SEQ ID NO: 40 - DNA, 20 bp, Artificial Sequence
  Primer
ccgtgagggaaagatgaaaa

SEQ ID NO: 41 - DNA, 20 bp, Artificial Sequence
  Primer
ccttgcgctttttactccag